SEQUENCE LISTING (45 sequences)

SEQ ID NO: 1 - DNA, 1828 bp, Glycine max
  source 1..1828: mol_type = genomic DNA
ctagagcactcgtggcccgtaggatttaattgagttcagcaatcaacacgccaagttaaggacattagctagtataaagaaatatcgccagcgggagtttaaatttaaatgtccttaaaatctcttcaacttcaaatatgtaacaagtttatcatctaatagttgttttatgtatctgatctaattttctactgtatgtctgtatcggatcttattctaagaactcggagtaattcttctctatttcttatgagtaagaaccacatatatcatcactttcttgttaatttacattgatgatattattattggtagcattaatttatgagctcatccataaacaattgaatgtcaaatttgccttaaaagaacttgaaagattagattatattctaagggaaatatgtagagatccaaatggaattatatctagcttaaacaaactaagtatatctcccaccttctataaaggttcagcgcgaagaatgcttctccttgtccatcacctgtggtgattggcaaaatgatatcaacagattgagaagaaaaatatataacacagattatagaagagaaattggtgtactacaatgcttaacaaacaccaagccaaatatttcatattctatgaattggcttagtcaatacatacaggatccaaccgtctcctactctagcaatgtataaaatgcatctttagatacttaagagaaaaaatggattatggtcttcatataaaactttcaacagacgttgacattttaggaatttttatgctgattaggcacctaatttggatgatagaaaatctagagcaggataatatgtattcttaggagattgtttggtgggtaggtcctctaaaaagcatataaccgtatctaggtcaagcatagaagcggattgagcacttaatgatcttgtagaagaattagcttggattctcattgctataggaaattaagttggctatacagaggacaccaattttatggtatgacaacctcaaaccctcaatgctaaagccttggctttctaatccagtatatggtgataaacaacacaattactttaattctcttcattattttctatcatgacacgaggaaacaacttgagaaaatttataatttcataaaggcgatacatttcaagtattattttaatctaacggtagcttttataaaatagtcattatcgttagtgaatattaatgcgcatatttttcctaattacatcattagattattttatacaaaaagtaatactatcatgtcaaattctaattactccaatattgaaccataaatctagtgaatactcatgcacatattttatttttctatattatcttggcaaacatgtctcatatatgtgtgtgttgaagtataaaatcattggcgcataaataatgacaaatgttttcaaatttataacaaaaaaaatgaagtagttgttcagtcaatattattttttcatatttaagaaaattgctaattttgtttatccttctcccacagttttttttttccgttctccaaaaagaattgtgaacgttctcctactgcaccgaaatatgtattctatttcttccatacactatgctgtgcccacataattaaggcatgtgatattatgctttaatgtaaagccacctacaaaaatatacttaaagactactactacttagtgtaccgtggtataacaatctcaacagactttacttgaaagtaacatctaaagcacaacctatatatattgctctctttgcccattcttcctcatcctttcactcttctcttgaattcacatttcaacaaccatagcc

SEQ ID NO: 2 - DNA, 2974 bp, Glycine max
  source 1..2974: mol_type = genomic DNA
ggccgacaggattgccagctgagcatcattcaattaaacaaaatccttgaggtatgctggggtagtgatcctccttttaggccttgttcttgatggtgcttgtatttcctaattgctatcaatgaaccagaggggaaaaatgcctaataggattcgaagaaaaagaaaaatgataaaacatgaaacccttttcaaaataacaaaaatgagttttcgtgttactgataggattttggcagtaaaaggtaactatgtaaatgttagattaattctttgaataagacaaaggggtcttcatttttcttgatgactaagtgcacgtgtaccaacaacaattttttgctgaaaatatttacgttcaattgttcttattttcacagaaattgaagcatgaatttgtgtataatcttttgcgcaatactatttgataggatgaggacatggatgattttgtaactattatcatcttttttaaaaaaaaaattgaatttgtgcagcaggaattcgtactatttaacttttctcaattttatttatttgtgcgataatctaaaatcaaaatgatcaatagataaaaagaaatcatgaatgaatttttgatatgccattcttttcctttatcaagtaaaaaatctatatgaaaagagatttttttttagaattttattaagttacatttttatttgatttaaatatatttttaatcttttaaatttgggtaattattttttctaatcccaaaaaaagtatttttattagttcctcaaattttcaaaaaactattttgagtctctttcattctttacattaatggtgttataatttgtgacgattgtctgctgtcaaacatatttttttaaatttattttttttgaatgatttatgataatttagaactgtcaaaatatatatataaaaattacaacaattttatatttatgtttttaattatcttctttaaaattgtcagaatatgtaaaaatatcaccaaatcatacgaatcaaatattttgatgatttaaaattgtcataaatcatttttaaaaaaataaattaaaaaataaaatttgaaaattaaaaattgttacaaattgtgaagctatacatacattgaataaaaaaaattaaaaataacttttaaaatctttaaacttttttattttaaacatttaataataagttttacatacgcacgcgcgcaaagagagatatattaaaaaaaattataacttttttttgcttggctattaaataaaatttattatatttgagtataaaattttcataatatggttgaatcatttaaagaaaatttttctacacaaaatctgaacataacatttcatttaatcagatatatattacctggacacctctcaaccaatcacaaatttaaaatttttgcattataaaacatagtatataccattgttaagaaaaaaatgaaagcaaaaacttttgttttccatgcagtagaagaagggagaaaggagcaccaaccattgcatggcacgtggacatggaagcaaaggaaatccaatgagtgatcttggtcatgcaccatcatcatccacacagcgcttatgagatgctcttgttcatctcgaccaagcaataacgataaagaaagaagtttatggttatggaggcctcggcatccatttgcaacaacagacccttcatcttctcttcctctcgtgccataacacaaaaatcttgcactctctcatcacctatgcttgtcaaatccatggccactcccaagcctcttccctcagtctctagaactgtaggctctagaaaggtcagtgcgcacacacacatattctttttgttcaaatggtttgctatgaagtgtgacacttatctattatttctttactactaaaagaatgaaaagggtattgtgaatttaactgttttgaacaaaatattagagaagaaggaaacattgtaatggttattagctcttaaattattgtaggtcagtgtaatatatatctatatatgtagaaaacttgcttacacaaattgttctttttcttcaaatgttttgtgaaactgaaacctgtctatagtgtttgaatttgatcacttggaaaaaaaaaagaaaatgatctgtggtgaattagctgatttgaacaaaaattacagtttaatggttattagtttttaaaatattgcaagtcagtgtaatatctgtctatatatctgtttatgtattaaacttgcttacataatgattctttttgttttattatgaagagtgaaacctgtctccaatgtttgaaacttattattaaaaaaagaaagaaaaatatctgttgtgaattgcctgttctgaaccaaaaaatacattatgaaaaaacaaaattgtaatggttagtattagctcttaatttttgtgagtcagtgtaataatatgtatctatatatatctatacgtattaaacatgcttacataatgattctctctgttcatattatgaagactgaagtctgtttctaatgttttaacttaaccactttaaaaaacaggctcttttgtgaattacctgttataaaccaaaaattacattggaaaagacattgtaacggttgtaagctcttaaatagaacttgttcaagatagtttccaacatatgaaagaacacctcgtttaaatggcataatggcatgagtgcatcaaataactaaatggcaaaacttagatgtagtttcatagttattttttgtgctattttctagtcaaaattagtgttttacctcagtaatcagcgtaataaaggtttgtattaaaggtcaacatacattaccaaagtgtaagtccaactctgatcggaaaacaacatcaaaaacacctaagaaactgtgtgcagtttttgtcctaaatgaaaaattgtttttcttttcatctggttaagtagtagtgtttagttacaataataactgaaccaagtgcacaatgcagaac

SEQ ID NO: 3 - DNA, 2778 bp, Glycine max
  source 1..2778: mol_type = genomic DNA
actcatgcctcctcaggttgtggcactctcttctttataattgtatttttaattttatgatttcatgatttttatttttaattttattttttaacaagtactaacttacaatcttactcattcacatataatctagacttgtagttctcaagaagctatgaccttaaattaagaaaatggtactatcaaacacattttctaattttctttaagaaatctacatggaggttacacttaaagaaatctatataaaactagctttgtgaaatttacaaacatgtatgcaagaaataattaaaaaaaaaagtggcttttccaaccaattcacaaaatggaaaggttttaatagaatataaaaacgattcaaatattgcattttttataaaaattttaaaaatattttttttgggtaattctcttttatatatgaaattgaagtgcatttttttagaattgaataggagagtatgaagggatgcttgagtttagaaactttgtccactctcccattagctacctaaggaaagggttgcaccacctcctcttttaaattgggtgtgtcttttaacatgttgtcacatcctcccgacttggtacatgcatgtcttattggttgttaagggatgtcaggagggtacaagagttgtctattgttggccaaggagctacaaggatgagtctagaagtcacttgttcatagtgcttttgaatcatgttgtgccttattgctcttataaattcccaaggttaggtcaacccctagttctttagtcattggactaatcttcaaggtatagttagcattgttttggatgacaggcttacagtcctccaaacactagacctaaggggtaggtgttttaagtcatcctcgtcctaatccattctatgcatatgtttttttaagcctaaaaggtgttttttttaaggcgctaagcctaaaagatgtaggtgcatagagtggtcattgtcgtccaatccatgcatgtgtcaatcggccaagaaccagaatgcaatttgaaaagccaattttacaattgttgaatgtgtgtttggatttccatttgaaacctttcaaacgaaaatttacgcatttcaaagtaacaaaagtagaagttccgtgttacttctgtgaaaaagtaagtttaatttaacgtaactttttcttaaacatcccctaaagccatatggaaaatggtatcaaacaaattttacaaatctatataaaactagttttaagaatttgccaattgtcgttaggtgtttgtactttgtaggcttgttgcacaaaatatatattttaattgccatctctcaatcaactgaaactggatacctggaaggctgcaatagaaaattaaactgcatgggatgcagctatcaatctatcgatgatttatatttagggggaactcttatgctttttttaacacaaaaacaaacattttcttttagtaaaaaaaaacagaaacattttaaaatgttaggatcagagcagttaaaaatttaactataaaacatcttgatcccattaatttgattattcaattgatttaattattcatttgattagttgtaccaaattaaaaaattaaaaaatctgcttaaatgtcttctctttcttcatcactgaaagtctaagaaggccatgtatgatttctgttatctgagtctttctatcatctgattcacatagctactactctgaatgatttgaagtcactgtttcattatatttgccaaacctcaacactcgttaaggctaaagtcaatggtttatgggatactattctttgtcctatttgcattcactttgagtttagcttttctactctcaaagtgtctatcaaaaagtcaaacaaaaaatgttccaaaagggtctttggggtaccctatcatcggagagacacttagttttctcaaagcacagagacaagataagggttctgtttggttggaagaacggatatctaagtatggccctatcttcaaaacctccttaatggggttccctacagtctttgtaattggacaagaaggaaacaagtttgtccttggttcaccagatgatttgctttcttctaagaaacccctcaccctgcgaaagatacttggaaggcaaagcttagttgaacttacaggaccaaggtacataattttcaacttaactcactgattcagcttaagttgtctgtttaaatgatttggatgaaatgtcaaaataattcttatatgatgatgatcttgattcaaattatgacactttgaaataaggtgtttaacctctgaatggatggattatggatagttggatagtggataagtgattttttggattagtggtagtatttagataactcattactgttataaagggacttactggtggtccttgtttcaaatttaatagaaatgaatttgaaagagttgtggttaaaaagattaatttgtgttggggaatgatatttataagtgagtttaaaagaattgattttgttttgaatactaaagtaaaatcactttagaagtgtaaatatatattttcaggtctgatacccaaaatcaatttatgtctaaagaaaagttagaaagtttcatcctctcaaaattatttctactcactgcaaaattgattttcaaaatcccaaaaagaaattggcaaaaaataatcaattcctagataatcaaatattatgatctccagtgtgattctcacgtgttacccaacaggatactagtcatctgttttgaggctttgttaatgtttgatgcagatac

SEQ ID NO: 4 - DNA, 2052 bp, Pisum sativum
  source 1..2052: mol_type = genomic DNA
cgtgcgcaacggcaaacttaacgaattctgatacccattctcgtactctctcgacaatcgattggagaacatccatgtattatccattactaattagaataaacaaaaaacaatttcagaagtgttcaaacacattcggacctaggtttctaatgatactggggttgacacaaaatcatatgttcataggtcgtataccctaactactgggtaatgtagtccattgcatgcgctatcatggtcataaaaaccaaccgtcatttcctaataaacccaaactatttaaatgactatgaatttgaaactttacaggtcgaacaaacgctagcatagacaacaataacaaaaactgaaattgggcaaagctaaaacaaagcaatagtgccaagtataatgtatgcatcgtgtacctttgattggtagaaggaggaaacgcgcagtaataatgtagatctaacagaggtggaaggaaacgccttagaaccctaacgtgaaaaagaacgaaaataacagatagtgaaataacaaaacgcgcagtatgttataattttaatgtttactaaaggggacattagagggcgcttgtggaaaaaagcgccctctaaaggggcctaagagggcgcttatggaagcgctctctaggctttccagaagcgctttataagctggaaatgcacatggacttataacagcgctttatataaaagcgcctctaagggtaaccttagagggcgctttctaaaagcgccatgtattgttgtccctctatctcctccttattttttcgtttcaccttagagggcgcttgtggaaaaaaaggcgcctctaaaggggcctaagagggcgcttatggaagcgctctctaaggctttccagaagcgctttataagctggaaatgcacatgactaataaacagccgctttagtaaaagcgccctctaaggataaccttagagggcgctttctaaaaagcgccatgtattgttgtccctctatctcctccttattttttcgcttcaccttagagggcgctttattacaaaagcgtcctctaaagtgcgctgtctattgctccttatttttcgcttcactttagagagtgcttttgtaataaagcgccctctaaggtgcgctgtctattccagtttttggtgtagtgaattaaaatatcaaaacattgaccacatatttaatattttaaattatcaaaaataaataataatgtacatagtaaaataaaaggggcgggttcggggacgagttcggggtgggtactagtgtccccattacccgacccatccccatattttaaaatcggggaaaacccaaacccaaacccagtcaaagcgggttttccccgtcaacttcgggacaggttcgggttggtacccgcgggtacagattatattgccatgcctaatgtcagcccctttgtggtagttctccatagtgataaccttgtaaacggctctcgcgttgacttgaatagcaatacggttaaaattcttcatgtgagctaatttaattccctcgagaactccacataattaaagattcatcaccctattccaagtcttttgatgaatcagcctagccatcgacctttgtatccagctttgcccaatgctcttcatgaggttgccactttatgtcacagattttgtactacattttgagtgaatggaaagaattaatatgccaattacattaatggataatggatagtttaaaagtcaaaaatcaacaagggtgttttaggaaaaatggtagtatcttttttttattggtaggtagttagttgatttatatatgtgtgaataattttgttttcattgtaacaaaaaagtcataaactcagagatcacatggtgtggcttcatgattgtattgcttaccacttgcctaacttaattattttcattcatcgaatattaaacaacatatcctctctataaaagcccacgctcacaccttatcttcttcacaccaaaattatccaactagttcaaagcctaatccatctttctctccttaattagcaaaacaacaatc

SEQ ID NO: 5 - DNA, 1966 bp, Pisum sativum
  source 1..1966: mol_type = genomic DNA
ggttaggtgtttcaccattgaggagcctcttttcagatttgtttgaagcatcggggagtaagcaggtcgtggttgaagattgtggcaggtgggtgggagaacaatggcagtgggagctgagggggtttaaacttggaattaggaggtgacatggaagaggactcgcgtgacctcgctttgcttctgtctgaagtaaagcctactcgtggctaagaggatgttgttggttggccaagaggtacatttaacatattttgtgtaaaatcttgctatgatattctcaatgtgttgtaggttacatcccattgcaaccaaattcgaagttggcaatggatattttgtggaagtctagggtccctttgaaggttaaaatctttggttggagattgttgctaaatcgtttgcctacgcggtcaaatcttgcagatagatgcgtgattttaaattcgcaagacaaggtttgtgtcttttgctttcatgttgtggaaacctgtcctagaactagaatggtttgaaaagaggttgagagttggttgggaatttttattccagctgtttcaaattgtattgattatatgttggcctgattcttaaaagtgatagggaaattatcgaaatctaatgcttgcttgatctggttggcaacatgttgtctttggtggacaagaaatcaggtgattttcaatggtgtcattgatgtcccaaaagaactatttcataaagttattcatttctcttggtggtggttggatttaacttctatgaacatacttaagtgtaattgctatgagtggttcaaaagtccaattgattttttttgtaggggttggaagttgggggctggagtttattagtttggttttgcatcctagtgatgtttatcattggtttttaacctgttgggttgggttccctttggaccccctttttgaatacactattgattataaaagaaaaaacaaataaaaaatattgtgattgtaagttatagtagaagagaagacgacaaaggagtagaaaggaatgaattgattagatgtgaaattattgaagttggatagtatagaagaagcttaaatattaaatagccaaatacaatttctatgtgaaatattttctttaaagaaaatttcaaaaaaaagttagaggattagtttttttagaaaatgaaaagagtataataaactaataatatttatataattataatttattaacggatacggatattcgcagacatgaatatcattaaacttacattcttatctgataaaaaataaatatttaaatattcattttgtttatcaacaaatatcaattaaactacctgtcctgaatttgtaagagatttcatcgacgaatacttacgcgtacttttttttttttctatccctaatctttcacccaatcaatctttttttctaattacgaaaagtttttcaagagatttctttgacatggttattttctaattaagctcatctatataacacttgttttgtttcttctttcttcttttctttatactaatcatgcaaaattccgaaaacaattttcaatttctaaacattgaaacttgatttttgtatgacaacaataatacagcttcagccaatttcaaatacatactcatattatagatacataaatcatcagcaacagtgtttagattgtgtaatgaagcttagaatggatcttgcagacaaatactcaattaccgttgctgtaattttggttcaacaattggttgaaaagaacattgcttaaagttaaagactactctactttactcacaataccatattattatattaatagcatagtatataaaaaataaaatcacattttcaacaaactttacttcaaagcaatatccaaaacacaacctatatatacaactctatttgcacattctttctcatcccttctcttacattcttctctaacacattcttgcaaatcaatc

SEQ ID NO: 6 - DNA, 1794 bp, Medicago truncatula
  source 1..1794: mol_type = genomic DNA
gtatggctaagcaatgggttagattatgctatataaattttttaaaatattaaaaaggtgagatttcggaattttttaaaattttgtaaaacttagaatatctttatgttctacatgtttaattaaaaatcagttgagttttaattaaaatattaaaaatgtgtttttggaatttcgtatattctatgggggtatcgagactaatataacaattagagaagtgttttattgttgggactagaaaaacaacagactattatttggcccatgtcaaatgaactcccgcacctagtaaagacacccctcatattttcatcttaatccttttccattattctaattttcaattttaaggtgaaaactcacccacatatttataatcttcctccatgcgaccttcattttccctttcatggcaaagtatgttgtgctctacctctatttgcgacgactctgtccttctcagcttgccccctcttcaatctatccctagctctttctctatatattttgtctcgtgtttggatcaaatgcatcaactggaatgttgtacaacacattgcaaagtcattggattcaagatgaaattttttaaaactatgttgccacatgtggtggccacattagagtaccaaatctctcggcttgggttcgatcccaatttattttatttattttcttacttttgggatactaaacttttcgtgatgctatcttaagcttatgagttatgtgacaacatgtgattttccaattttggataggacaatgttaaaaattctctaaaatatgtttatactagtaaatatcatgcttttagggagtagttgttttagggagaattatttgtatagataaaaaaaaacaaacattttgataaatactttttttttttgacagggagttttgctaaaatacttagtatccaataagattgatttggtataataaaactttgtttattaattaaaaatagtaaaagagattagaaaatagtcaattgaaaattaaaatatttaacctttttaataaagaatttctttatttacaattcctaaataatgtttttaggacactcgttaacaagattgaaaatttatttatgtttcaactattttcatatgtttgtataaaaaagaagtattttcatatgtggaaaattcatggtttttcacattttgagatatacaaaatatgttcttttttttttttttttttttttttttttctttctatatttttaatactcactttctaacccgaaaatgttaccgtgatacgaataaaaatgtccagaaatattgagatattttcttttgtgaaaagtgtggaggcaaaatgtatggggcatggggacttatttggggacgtagagtaaaattttcctttattatttcttctggaacatttgctatttgcaaggagatgcacatagaggtggaaaatgtgtcttgtgtttagaatgtctatgtaatgtgaagcttagaatgggatcttgcacacaaatactcaatcaccgttgctgtaattttggttttcagattgtttgcttgcttgttctccaattggatatcaaaaacatacttaaagttaaagactactcaactcactataccatattatatatataaatataccattgtatataaaataaatattccaacaaacgttcctttaaagcaatatctaaaacacaacctatatatactactccctttgcacattctttctcatcccttctcttatattcttctcttctatttaccaattcaataacacatattgtttccaatc

SEQ ID NO: 7 - DNA, 2266 bp, Pisum sativum
  source 1..2266: mol_type = genomic DNA
gggtcaagtggagtggatttatgatggacgaggccttagtcattttgggccttttccctgtccataacaattactggaaggagaatacctagaaagactatcaaagaagttattaagaatgatcccgagattaatgatttagatagaagtatggtccttgatagaacattttattgaaagaatgatctctgccataccattttgttgtagtgtcttgcgaattgagaaatatatttgaatattgtgttcttcacataacctcttgaatttaaaattagtgtactatgtcccaattatctaatagcaaatacttgattttactgcctgtttgattctctacccctgccttccacaacttgaatttgataaagacttcagatttttgtttcaagaaatacacccaaacattgcaagaaaaatcattgctgaaagtcacaaaatacatggaacttcccatggaaatctctttggtaggtccctacacgtcaaaatgtatatagtctaaaattctctctattatgtgctttctggtcttgaatcaaacacgacattgtttcccaagtatacaatacttgcacaattccaacttgcaactacaaacacccttcaatagaattctcttatgaagttccatcatcctacactcgttgagatgacccaaacacatatgttagagtttggttgcaccattatcagaatcaactaatgcaacatcacctacaacaatgtccctctaaaaatttgtagacgttacctacaatcctccttgctctcatcacaatcaatgcaccctttcttaacttcataatgtctctattttcttcagacctagaggagaaacgatttaccagtgaaatacctaggaaaatcaaattcttcctccattctaaaatatatctgacttcgttccatgtttgcacgccaccatcaccaatcacataatttttcaaaaaatatatatatcaaagatgttttttataaactacttaaaatagttttaaattttaataactttttaaatatttttatatataaaagtttatagtaaaaagatgatataattaaaacaaatttttatgagaaacggtttaaaatcaatttttattttaaacttaaatttttttttcaaaaaaatgtattaatataaaaattattcttaaaagaaacaatcaaactctcagtttcaaaataaaatattaaaaatcagtttcataaaaaaattatgttaataaacaatgatgaaacgataattgtaaatattttgcaacaaatttaatatattaggaactcattaaaatatatattttttaaaatacttttaatgcaacttttttgggtacaaacttttaatgtaactttaaaaaacatataaatgaaaaaccaatccatggactgggttctgcctcatcatcattatcttcatccactcattaccatttcttaggattctcttgttcatcttaacaatcaagagataagagaaaccaaaaaaggaaaaaaagaaagaaaaacttatgatgatgacaatgaaaacctcaccatccatttgtagtaacagcttcatcttctactcttctccatcaacacaaaactctcacactttctcacctttccttgtcaaatccatggctactcccaaacctcctccaccatctgtctctagaaccgtagcttctaaaaaggtcagtgcaaaataatcactgcataatcaatatattttccttctacttgttattatgatttgttataatttatatagtttcttaaattttgattccttatatgcaatattatgaggagtgcaactgtggaacatgtggaacataaattgttattgatatgtaagaaaaagattggtataaaacatttctactctaagtagaatttaacataagcatttgaaatgatattgcacggattcgaatccgcattttagagccgtctaaagtttttagagatacaggcttgttaactatggtttatggtttaaccctgacaagacaattgaagatttttttttagttctttagagaaaataattaataatgagtagaaactcatacatacaatcgtgactatcagaattcaaagtctggtgattgcgtataacctatcaatatcagcttttttctgttatgtcagagcccgacaattttaagactatatgctttaatccacctgcacacttttaaagacggccctcttgttttcggtgtcaaaattttgatttttaactgttgctaattcactaacacaattgcagaat

SEQ ID NO: 8 - DNA, 2924 bp, Medicago truncatula
  source 1..2924: mol_type = genomic DNA
gtctccatatggatccacgttggcattgatatctcttcaatattactttgacgtcttgattctatcgaagggattctgctcaatctcaacttcattttgaatgattctagcatgttacttggaaaaaagattacaactcttttttatcctaactatagttatgactgatagtaatttgtgtttacatgtgtgtcgtgtttttcttccccattccaacaatacaacaatagattttcgaggagtgattcacgtaaaggataggagagtgagagattttcataattttttgacagtaaatttgactacttcgtgttcaatgatttcttcattattccaatgcatttggggatctttattttatttttttatgcttctccagatgtttatgaatatgtatgttttgtgagttcattttcaagaatagaaattattcggagatcaaaggctaagaattgagtacttttaagttatactctataattcggaaacaatccaaacaccttcgtgagtgaaatcattgagtttcttgataacgtgaagagaatcaagaaaatggtagaacttcgagtttatactttaagactaatttcttgtaactcttctatttttttgtaagagattctttgatagtggattgaagagctctctctccaatagacatatgtcaaatttggactgaacttggtaaacaaatatcgttgtgctttttgcaaaaaattatttgtgaacattttttttttttttttattgtttgtgcttgtttgattgatgctaattttgcttcacacattaaagtatcgatgtgagttttgattgaattcacaacaattgacgcctactgtggggcaataacaacaaatcattaaacgagcaccatggttaccaagtggaatatcgaaaaattcatcggacgaagatgaagatgcaagcagtcttcatgttccaacaaaagtgtgcaggcaccgactgttgaggctgcattgtctgctgccatgtcaaaagcagacaaaactaagatggtgcacaaggcgagaagtgtcattgttttgtgcctctaagataaagttttgagagaaatcgtgaagtaaccacaacaacatcttgggcaaggttggatttgttgtatatgccccagatgttggctcataggcaattcttgaaactaaatcttatttactaaaaagtgtctgcgaaagtaaatttttatttttttaattttattattcgtaatagtaaatacaaattattttttagaataaaatgtaaaaaatttagtatgaaattattatttttattatgaaatgatattttctaaaacataaaagtccacaaataattctttattttataaaaataaacattaatttattgatctaaaaaaaaaccgatgtatgatgtatcaatacacttcaatttacgaacgtactagtaaaagctccctatttacatacatgtatcaaacataagaatgttaaattatcatccttactttctctttggtaaacttaaaacaaacacacatttaaaaatttattaaaaaaaaaaaaaacttcaaaaaataaatgacataatggaaaaccaatccatggtctctattgtacctcatcatcaattatatcatccactcattatcattgcttatgatactcttcttcatcacaactactaagtgataagaaaaaaaaagaaaagaaagaaagaaacttgatgatgatgacaatgaaggcctcagcatccatttgtagtaacagcttgatcttctcttctcctccctcaacacaaaactctcacactttctcaccttttctggtcaaatccatggctactcccaaaccagttactgtcacttctaaaaaggtcagtgtaaaacaataactatattattacttgttattatgatttttgatgatttacataattgcttttattccttttatgcaaaatattatgctgtgtggaactggaacatgtctataatattatattatttaccactttaattatgagaaaggttgaaaaactagctcttataatattataaaacgtatgtgaaatttaacaatatgtgacatcggttgttatgggagttaattgatctttaagaaacaaacttgtataaaacttttgtcgcgtaagtacaactcatatggtaaaagactgaggagacatttgattttctcagaggcgggtttgaacttccaattccctacttatcaacacttcgtgtgtgtgtgagttttgtcgcttgctggactatttagggaaaataaaggaactatactgtcaaaaaatttcaataatttaaccattagcctacgtagaaattttactttgtcaggcttttaagtaggctaacacgccaggccttcgaaaaggtcagacaagtactaaaaatggtgtatgatagtgaatttatcacgtttagaatttaaattttttaacagaccaggcatatttaaacaaagcatttctaaatatggcattatgtaaaacatgttttttgagaaaattctatggtggtccagttagataggtgtccaaggtggattaacacttgaaaataacccgaaactatgactcatggtacttgtcggaagtagaaactcatgatcctctttgtcttcatgagtgagacggtgacttgattgatactagttcgaaaaaaacatatgggaaaacgaatgagccaacaaaactcaactcatattcgtttttctatgttttggtagaacacgcaccaatcaagtcaccatatcacctatgacagcaacgatgactgtgattttctacttccaagatcaacatcggttgtcattggtatctctacgataattttcaagtggtctaccgtagaaccagcctgttttttggggagaaaagttttatttcttacagtaactaagtcactagcacaattgcagaat

SEQ ID NO: 9 - DNA, 2608 bp, Pisum sativum
  source 1..2608: mol_type = genomic DNA
agtcagggttcgaacctcgaaccttgatactgttgtggtaataatcactaaaaattcatacacacaattatgagagtcaaggttcgaaccttgaaactcctgtatgaataatcactagaaattcacacatataactttgagagttagggtgcgaatcttgatactgccgtagtaataatcattagaaattcacacacacaattatgagagtcaggattcgaaccatggtgttgatgtagtaataatcattagaaattcacacacataattatgagaatcaagattccaaccgtggtgctgacgtagtaataatcattagaaattcacacatataattatgagagtcgaggttccaaccatggtgctgacgtagtaataatccttagaaattcacacacttaattatgagagttagggttcgaaccatagtgttggcgtatttataatcactagaactcacacatacaactgcgagagtcaggtttcaaaaccctgattgatgttggcattgaagctaacaattttcaatttaaatgtcactttaataaagaaaatttaaggttctttccataactataaactaacaaaatagatgaatggttattggaacataatttgttgacataggtacatcgaaagagcagggttactaggatgtttgaattggaggtatccaagtacattaaagcagacagaactcaaatgggctgagaaaggatcaaggaagaatggtgacaaaggctatggtcatcatcaatcaagaccaacaacacctacaactccaacaagtcctttctctattgctagcagtgaagcctcctatgccatgtctgcaacttattctcctttcactcctcatcatcaccaccaccctctttgaatacaagaagaatcaatttagctttaaaaatagttttttcaactgtgttttctttttatatttgatcttatgctgttttattttatcaactagttgatataacttatattaaaagttctatcatttcttttttcttatctgtcactttcttcaatgtgtcgtttacttgcatcaaaataatcaatttataaacgacaaatagaagtttggacaaattgtatagatcaaggcattagattattcaataaggagaatttaaatattaaaagccatctaagtttgccggccaattccttaacccaataaatatcatctaataaaaaagacaatgatatttgaaactatttatgtcaaaaaaatcatacttttgattaaattgagtatctgttgcatgtttctgcagagactaatcgttcgagtcgagtctttcaagacgttttaactttgctgcgtgtttaggactaaattcgaacccaaattcacttgtataaacatttatcatcttacataagattccatgcgttaataactctatacatattcacgtggtaagtgatctctttcgatagtttgagattgaacggctcagattaaattaacgtcaaacaatttcaagtgttgatttgaaatcaaacggttaaaatcgaaaactacagcgatgtgtccatctaaatgaataaaattattcatttgattattggtaccaaagatgaaatatatactaatgtcttttttattccaatgtctcttggtgacataaagtaataaaaaaaatatatatgatatctatctcctatgtttggcacaacaccaaatgatctcaactctctcgtataatttctaaatggttaaggatattcctttcattggtttatgtatactaactttgagtttagcttttgtcttgaaaaaacttctctcaaaaagtcaaacaaaaaatgttccaaaagggtctcttggatatcctattattggagagacacttgagtttcttagagcacagaggcaagacaagggctatgagtggatacaagaaagagtatctaagtatggttctgtctttaaaacctccttaatgggttctccaacagtgtttattattggacaacaaggaaacaagtttgttcttggttcctcggatgatgttatttctgctaagaaacctacaactcttcaaaagatacttggaaagcaaagcatagctgaacttgtaggaacaaggtacatattcttaaattattacttatgatcttgttatccagccaaaaaatcgattaatcttggagatcagtcgattttttttagagttgttgatttataaatatcataaatttgtttagatatatatataccgataccatattccgtaaccaccatatttttaaaataaagaattcggagagaaagaatctatagaactttggaagcatgagtgaaaactcacaaatggaattatattgttaaaacatgttgaaatgggaatacaagatgtataagaaggttaactaactaattagttctcaacaaattgaaactataactaactaattattccctatttatccatatacttatatgtaacaaatagtctacacatatcacaagaagggtcatatctagttttggacagggactctagttttggacagggacacagacacacctttttttccagaggtgtcgatgttatgtgatttgagctttttgtttattttttcatgcaggcac

SEQ ID NO: 10 - DNA, 2702 bp, Medicago truncatula
  source 1..2702: mol_type = genomic DNA
ctgctaagaagtcgtcacatgtcccaaaacaagagttataacataactttttaataagataatgggtcaaaagatcatattcccttatgttaagggtcaaaatagcatatttttttaaacatagcggatcacttttgccattttttttagggttaaaatcgtaactttttaaaatttaatgggcgaaaagtacaattttgcctaaacattactatgtcttaaaatcgtaacatttttaatagagttactatgtcttaaattaaatactattgatgtatttttcattatcataaatgcaaagtgaaatatattgaattaagagtgataaaagtagtattaattagagttataattaaaacaaataattaatattatattgaaaagtgaaatgaatcagttattttaggaaaactttgttatgggacgaggagaatatttttttatgcctccaaatgaaaacattttacgtatatacatttgtttattcatacaatcattattttcatactttcttaaaaaaattattttcatactgtttacacaatcttgtcattattataagaaaaaaaatcataattttatattcatttattagatgatgtatgttgtctataataaagaccacataccctcttttgattggacaataacaataaaatgaaagaaaaaaggagagaaaaaataaaaatacaatgaaagtatgttgtcttaaaaatacaataatatcatttacttattacgataattacttttgaggtaaaatgtatcacatagaacaacactgaaaaaaaaaacaaaaatatatcatatctttgcacattaataaaagtaatttatgctcgtgagcttagttcaattggtaggggtatcacattttatatgcaagagtcggggttcgaaccccggacactccactctccacaattaaattgtgtgagctctagccactaggttacttgataaaaaaaaaaaaaagtaatttttgtttatttccataatgatgtatttcacttcatcatttgatacttaaaataaattaaaaattgtttacttagactaatagaaaatcatatatttacaaataacaaaaaaaaagaccgagtaagaatcctagagttagagttaggggaaccctagggcaacctttgggtaactactcgatgttgagtatgaatgaaacacaattaatccttcaattttgattttgatgacaacaaggtataagaatataattaatcctctaatatttgtttcacgtgtaaaaagtcattgaatcacatgactacaactttaattgtaattatttttgttacaatatggtccctccatttcttcaaaaaaaaaaaaaacaatatggtccctccataattatttttttttggctccgtcaccatcaattaatgataattataatgtttccctttcaacttaaaggaagcttttagataaatgatgatgtttccctttttttccttctaattgcacgaggtgttcctctcaatcaattggattatacgcctgcaaatataaaatgatgtaacttagaaatctcaacattaaatagagtagctataagaaaacgatatatatttgatttcaatcaccagaaataactactactttatatgatcttaggtctctcaatctaattttccaatggttgtggaaatatatttcatagttatatgtctactcactttgagtttagcttttctcctcacgaaatttctctcaaaaagtaaaattaaaaatgttccaaaaggatctatgggatatcctctcattggagagacatatggttttcttaaagcactgaggcaagacaagggctctgagtggttagaagaaagggtagctaaatatggtcctgtcttcaaaacttccatattgggttcccctactgtgtttattattggtcaacagggaaacaagtttgttcttggttcctcacatgatgttctttctagtaagaaacctccaacacttcaaaggatttttggaaaacaaagcctacttgaactcacaggatcaaggtacatattcctcattgatgcaacttaaattgttgatttaagtagattagattatttgtcaaaaatccttatatataacgatcaaggttttataaaacgatttgtgattgcaattgtggccgcaaccttaacgatcgagggtttataaaacgatatgtgacaccccctcacactcaggattgcacatctgaagcgtgaacataaatgacgggtggtccgataacggaaacctgataacaggtggtccaaagaaacgcgaagaggctctgataccatattagaatggtggttgggcctaacacaaccccacaaaaccggcctttgaggtgaggattgcccccacttataaacaatttgtcaggccaattcctgaccgatgtgggactcttaacattagttagttacactagttagatagtactctaattagttagttagttacactactagttgtttttctcatttgcaaaaaatatcttatgtatcccttgatgtgatttattttatcaatataattttaaattttacttttctcttatcgatgtcatctatcaagaatagcaatatgatattagaatatgttttattctatcaagaatagaaaaatgaaagatcgcgatgcaactgtgatcataatttaaaacctgtgtttattttttgtgtaggttc

SEQ ID NO: 11 - DNA, 3489 bp, Medicago truncatula
  source 1..3489: mol_type = genomic DNA
cgcatacatctagtcatatctcatcgcagatatttgtggagacataaaattgagatataattagatatatgtgtagattttttatttatttttttatgattgtttctgcaaattatgagatattaatgtacccaaaactattgtttctttcaaaatattctttcaactgtttgtttttttatttgatcttatgtttctttattcaatttgttgatttaacttgtattaagagttctatcatttctttctttatatgtgtcactttcttcaatgtgtcgtttacttttgtacctagctaaaaatattaaatacatatacaaaagttatttgcattaaaataatcaacaattgtcaagtttacaagcattgcttgatagttctgttttaataaacgacaaatacaagcttgtgtaaattgtatagatcaaggtgatacaataaataatgataaaataaaatacgttgttaagtctgccggccagttacttcacccaatacattgttatctatcaaattagtaaaaaaatgtttctgaatttactactgtcaaaagaatcatggaattggtgcagtaaaaaattagtaacatttgattaagatcagatggctcacgtgctaggtttgtattttgaataggatttattaaaataggaatgcaagtcactgatcttgatcaaatagtgctgcttgtttggctgctacgacttcatatttttcttgacctaggaaatcggatgtggataatttcttgttgtttcatgcggtaaacaatctctcggaggttaatatgagattagacgactcatactacacaataacattatctttaaacgacttcatgtattattttggactcaatggtgaagtgaaaattgaaataattaataactatcgatcagactttacttctcttccaaccgcactatcatgatcaatacgttatttagatagacctgctctttatttagagagatacacaatacccaccccaccattgcttgaacataaaaaaataaaagccattttgctgcaatgtgatatattttttatatttttgcataatgtcatcaccgtagacccaaagattatatatccaaattaaaaactgctgccatgtgtctattaattacaatatatatagaagattctcttatatattgtaatagtgtccctctcaatggataactagattattggcattgaatataaaatgatgtaccataccaaattaccaatgtcttctctttccaatgtctcttcattacattacataaagtaataagaaaaccatatatgatctctatctcgatagtttagcactacaccaattgatcacaagtctctcgtctaatttctaaatggtagaggatatacctttcattgctatatgtgtactcactttgagtttagtttttctcctcaaaaaaattctctcaaaaagtcaaacaaaaaatgttccaaaagggtctttggggtatcctattattggagagacacttaatttccttaaagcacaaaggcaagtcaagggctctgagtggatagaagaaagggtatcaaagtatggtcctgttttcaaaacctccttgctgggttctcctacagtgtttattattggacaacaaggaaacaagtttattcttggttcctcagatgatgttttgtctgctaacaaaccccctaccattcaaaagatacttggaaagcaaactctagctgaacttataggatcaaggtagatactcttcaactcttaatcattgttgcaacttaaattgttgttttaaaatttcaaatagtttagatagcttgtccaaagtgaaatggtattttaagggggataattaaatggcatcatttaaacaatatttcattgtggttaaaatctcgatcagcattctgaaatcttatgattttgtgattttgctctgccttaccgatcagaatcctgagtagaatcctaaactatgtaaaatccgccatataattggcgcgttcgtgatgtgatgtaggcttgtaggatctagattttgagtatttttttttgacttggttgttttatatattagagatttgggactttgagtcgtttattttaagatgtgaaacttggttgttttatgctaattttaagatttcaaactttacgtggcttatgcacgttatagcaaagttgttaaaatcaggattttgcttcaagtcgaaagggaagagcaaaatcgaaaatcttaaaatcgcaatcaaaatcgaaagattttactcaactacttttgtaaaatcgatataagaaatcgtaaaatcacaatcaaaatcaatggattttaccaaaacaaaaacaatgttaaatatatgttataaattatatatagtcataaatgactcggtttaaatccacaaaatacttatacataaatgactaaaaaatggacaaaatccttaaaaccccttttcaattcctgaatttttgtatggatttcacgattctgtgctattttactaggattttacctattttgggattctgctatggatttgagtcatagaaggtgaataaagtcgcaaaatcctgcattttacgatttaagtcaggattttgacaacaatgctttatagtatatatttaatattatttgattaaaaaccttcgattttggttgccatttttggttttgtgatcttgctatcctctttcaaagttaagcaaaatcttgattttgacaacctttgttgacacaattatgtacagttttgttttatattagttaggaactgtatatcaactgtgtgtgaagcatcttatgtaaatgttttcctatttttaacatacaaatttaacatcaaaattcaacaacaatttagatgcttaacgcaatttaaatatgtggctaattatgattagtagatgattaatgaatatggaagatgctaaaacttattaaacacttactcaacacaaccacacagtattgatcgttattaatcacaaatttcaagtgtatatttgaactcaaatagtggtcagtgagtataatgtatagttgaatatttgagtggaacatgaagttgaaaattgtgttatagtgaatggttaataagttaaaactcctttatttagcatatttaaccgcgagtttgaattgtgctaactatctaagttgatgtggaatattgatgtcaaatttgtatgtcaaaataatctttattataatcaaggtttttaaaaactcttgcatgatccaaattgaagcggcaacatcaagtttttctttattttttatgtcaacacatttgaccaaagttttgcataagatcatgacgcaacagcgacaacagtttcaattttaaaccttgattgtgatcttgattcaaacagtggcgctgtgaaataaggtctttcatatttggacacacaatttgaagaattgatatttataaaaggatggtgaatccatctagtttgaggcttgtgtttactttttgtgcaggcac

SEQ ID NO: 12 - DNA, 2050 bp, Solanum lycopersicum
  source 1..2050: mol_type = genomic DNA
gtccaaatgtagtaatgtgcttctttaattaaaacatacttaacacactaatgggattaatattattccatattagtggcaaataattaataaaatattgatcatgttattcatgacattttaatatcattcccttttaatggtagccattcaacaaaataaatatggaggacccataaaatcccactgacaattataagtagagccccatttcttcatgaccattattttctctaagtactcactatgctcacctcctttaaataaagaagaaagatgctggggtttatatatttgacggaatcaaaattttcattaaaagaacgtaaagtattaaagagaagtaaaaatatgaagaagtcaaagaaatctaacatgcatatactcttgtggacgtgtttggtgcgaaggaaagtatttttcttaaaaaaatatactcgtggaaataatcagatttcttacttattttcttgtatttgatctatatgtaagcaaaatttcttattttattgttttataaatattacatagatgtatattgtaggagttcgacggtcgggtggaaatgaaatgtgttagaaagtgaagaggacaaatattaatgtggaatattttttgtagaacttgtttttcctagttcctctaggaaccagttaaaaaatatatcttcatagagtaaatatttttcttttcgaagtatttaagcaaccgaacattgaaattttagttctccgcataaaaacaacccatttttatacaattaagaatgattttatttttttatatatataatataattttttgattaaaaaaattcagatgaacctattacgctctgtctactccaaactttacatatttattctttttttttttgtctgatgtgctaaaaagtcttaatgaaactgaccaatacaattgatacttttttaatccacattattccttaaaattgctaaaataattgggttgggaagtggggactgggcagggccaaaggaattgatgcacatacttctgatgtgctatatattttttttgttaatttaattaattatattatactgtaaatgtacatataattatattagactgaatgattaatgtgaaagagaaaaaaaattcttacaaaatcgattatcaagaagagatatgttgtttgctgcttacgtaaatatctgacctatactatagtcaagcaatttacatatattattattacgttcatcatatagtaccgaatataataacaattcaacaataataggggtggtttaatttcccatgaaaacatgatgatccgtctaattcgatccgttctaagttttggcggatcattaatctgtccatttataaactcaacatgtcttgattttccgctcaaattaagtctatttaaaaattcgattgacttagctaagtatataatctaaactgatcaatgagaagtatttatgtaatttttttttgtttgatatgttatataacaaaatatcattacaataaagaaaaatatattttttaaattagatttgtttggtaactaaaacatattataaaaaaaaattaaaatttggaaaaattgatcatattggaataagtctcattattcagttcatttcagctcaaataactttagaacaaaattgacaattcatttattaacacattccattttatctcgtcttattataattcagtccaaatcacacatttatcatcataatattatagcataaaatcacatatttaatgagaagttgaatctttaattcaaagccaacaaaaaaataaaaataaagcaccatgctttaagtctcctattactactactccatatactataaagcatatacaattaatatttttgcacaaaacttagctttaaagcaaatttatcaaaaaatttacaacttatataaactcattaattcccctctatttcttcattcatctcaacacctctctatattactacaaaacaaatataaaaatggcttccaaattctcatttcttatcctatttcccattttattactttctctcatc

SEQ ID NO: 13 - DNA, 1929 bp, Arachis hypogaea
  source 1..1929: mol_type = genomic DNA
cttgagcgttttgggagcaaataaatgatcttgttctgtgtcatttttataatagatttgtttgggtgaattgttaaaaaaaaattttgaagtaaatttttttaaaaaataaaaataattttatatttatatattttatataaaaatatttttttatataataattatattcagatacaataatataaaaataatttttgtttatttattatgttaaaaatatattttttattaaaaaataatttttttaaaaagtataaattatatgttttaaaaaaatatgttgttttgatttttttaattttttatttttactactaaaattttgtcaaacatattaaaataaaaaatatgaaaagaatatatttttctaatgacttaaatgacattcaaacaaataaattatcattaaaattacttttttttactaatatttttattgctgatgtgaaaaataatttactcatcaaagcaaaaatactaagtcactctcggaacatcttaaaatttctcattgtattgtatttggtaagagatgagtggctacaactttatagttttatactactctctctcccggtatttttttttttttttttggtgactactctctctcccggtataacagtatatagtggttttttttatgacttaattgcctttatttatacatatggtgatggaaagttctcttgtaatcaagaatcttatatactctaaagcattctcagactaatttgaatatcctccatttctgctacattcagggtcccccttttttaaaaaagattaatagtaataaataattaaatataatttaattttttaaaaaatttatttaatatttaatttaatataaataaaagtctaatctaacttaaatattaatgatttctaatatctaaaaagtaataatattaaaaaatctaaaaagatattattactaatatttttaaattaaataaaattttttaaatctcataaagtggatttttttttgtaagcgtctttcttgattcatttggtattaattctttatgtttcaactactataactgagaaatctttttcaactataaatgttgtgaaaaataactcagaaacaaaataaaagatgaattttttgctaattattttttaattatattaaaaagaaaattactaaaaaatttgatacagattatattatcgatgaattttatgatacaaaaaattgaccactttgttaataaaaaatacatatatatattttttatattttaaaatatattctttattgatatatttttataatatattttatattatataattttttacataattttttaatattatatatattattgggccatgataatatttttggatctctctcatgttataaaaagttaattttgggttcaacaagtttgaactcttgaccttctggatttggaactctaataccatgttctgaaactactcatcccaaaagtgttacctgataggataatgtaacactaataatcatatctctaatactttctaaaccttcattgtacatattgtacgtttaggtcattggctccctatattttctcttacttaaaatatagagagaatttaatatatatacatgacgtatgaaggctctcctactgcaccgaaacgtatacattgtaataacttagattatattggccacataattaagccatgtgattttatgctttgatgtaaagctaagtgaaagtaccaatcctaccttaaagactactactactactactcttctaactcgatcatcggtatatagcaattcccaacaaactttacttgaaagcaaccgcacccgatgcatatatatatatatacatcattccctttcttcattcttcaccattccttcacttcacttcactgttgtcttgaattcaattgcatgcc

SEQ ID NO: 14 - DNA, 1809 bp, Arachis hypogaea
  source 1..1809: mol_type = genomic DNA
ccccttagccttagcctgtcactcttttcccttttatcatctctctctttctcctctctccccctctttaattttacaccattattgaatcaaattatcctctcctctatggctctatccatattttatataataacatgtttattattacttcaaatagattttattaatatacctaacaaaatactagaacccacccaggtcaactactatataaatttgatcacatacatacagatatagtttttaatttaaaattgaagtagttagtgttaattatttctgaaattcaaattctcttgcgtaaagaagcctatcacaagtataaaccaaccttgttggcctcttatttttcataaataattacacatcaggcctaaaccaacaacaggataagttattgtatttttttcttttttggggtcatacaaacacacactcactcacatacactaacctagccaccactgttaggatttaaacttggtgtggcttagtttgcctcctttaccactacatcgcatgcctcagccacggtccacggataagttatttttttttggtacttgacggatgagttgttgtttgattgtctctaaggtaccacttagttactgggccaagtctaatatcacttcttaggtttaaaggcccattgttctattccactatttcctctcttttttcattcacactttatggctgtatgtattgcactggaaaatttcttgagcgttttgagagcaaataaatgatcttgttctgttatttttataatagatttgtttgggtgaatttaaaaaaaaaatgttttaagtaaatttttttaaaatttttaaaaaaataaaaataattttatatttagatattttatataaaaatattttttatataataattatatttagatataataatataaaagtattttttgtttatttattatgttaaaagtatattttttttactaaaaaataatttttttaaaaaagtataaattatacattttaaaaaaatatgttgtttgatttttttagtatttactattaaaattttatcaaacatactaaaataaaaaatataaaaaatatatttttttaacgacttaatgacactcaaacaaataaattatcattgaaattacttttttttaactaatatttttaatgctgatgtgaaaaataatttactcatcaaagcaaagtcactctcggaacatcttaaaatttctcattgtattgtatttggtaagagatgagtggccatacaactttatagttttatactatctctctctcccggtataatagtatatagtggtttttttatgcctttatttatgagaaagtatggtgatagaaagttctcttgtaatcaagaatcttatatactctaaagcattctcagactaatttgaatatccttcatttctgctacattacttaaaatgtaaacagaatttaatatatattcatacatatatatatgtgacatatgaaggctctcctactgcaccgaaacgtatacattataataacttagattatattggccacataattaaggcatgtgattttatgctttgatgtaaagctaagtgaaagtaccaatcctaccttaaagactactactactactactactactactcttctaactcgatcatcggtatatagcaattcccaacaaactttacttgaaagcaactgcacccgatgcatatatatatatatatacatcattccctttcttcattcttcaccattccttcacttcacttcactcttgtcttgaattcaattgcatgcc

SEQ ID NO: 15 - DNA, 1896 bp, Cicer arietinum
  source 1..1896: mol_type = genomic DNA
gcatgaatgcatcagcccgctaaaatataggggttgggcctaattattttaacccgacaatcatccaacatttaagaggagaatcatccaacattttaaaaaatatgaacatccaacattttgaaaaatatgaacattgtttaagtagtttaaaaacacttattttagagtcgcattataactatttaacattttatttgaatttggtttgagtttaggttatattagacaaatctaaattcatttgtgttatgttagataatatctctaactttttgacaatttgaacttattttaatttgtgtgttatttagataatgtattgttttgaaattttgaattaagtttgtgttaattaaaaaattcaaacttagtttatgtataaagagtaaaccattatttttttaaatggacattcaaattgattcatacttaatgcataagtagtaaatcataatttttttaaatgtgcatccaaaaaaattttaaagttaaaagagtcgcaaataaattaaaaatataatgagtcaaaaaaatataataaaaaatatgtggattacttgcccagcccacaacttgtctagggcagagcttgggtagtatttattgacctatattaaaacaaatctctttgcctcggtcagaagaaacccacaacttgttagggtcgactccatatgggtggggtgtcatgttttgacaactctaaggtagaagttatggatgggaatatattggaccacagcagtggttgttttgaaaatttaaaatataagtccggtctaattacttagcataggccatttttttagattttctaaaagtctattatgacatgaaagactatttaaaatcttattttttatatcaattttaaatacaataatagttaaataggtaaaggttaagagccatatcaatctacacctataccaaatataaatataagtccccaattaatttgctatatgcaataacacaatcgaaatttaaaaaaaataaaaaaaaatacagccaaatagacttttaaacattaatttaagaaacatattcaatatcattataacaagatcatagattttaaataagtaagtaatctatcaatttcaaccataatccactttaaattttacataaaattgaaatagcaaataaataataacaaactccattatatatataagttggtctatcaaatttaataaaaaatttctaaaaaaataaataaatttttaaaaaagtttaaagttgacatttttaataaacaagagaggtagaggtaaagcgcagtgcacgtggaagggagatgggaaatgatatttgtgaaaatatggtggtgtgtctagtaggtgtgggggtatatttagcatgggaaaaataacagtcttttgtttgtttaggaccaactacaaaaacatgttctctttcttctcctctctcttagtgtttcaattaacggtgtttgggatatgtaatattgaaacttacaatggatcttgtgcacaaaatattcaataaccgttgctgtaattttaattctctaattgtttgcttgcttgttttccaattggataaagaacaaacttaaagttaaagactactctctatacaacaatatcataccatattataaaattaaaataaatattccaacaaactttacttaaaagcaatgtctaatacacaacctatatatactactccttatgcacattatttctcatcccttcctttatattcttcttctgttctattaccaaacacattcttgcaatggcttttatatctttttcctccaaaacacttgttctagcttgttccttatgcttatttatttctttagcatttgcacgtgatttctccatcgtggggtattcatctgaggacttaaagtcc

SEQ ID NO: 16 - DNA, 1937 bp, Cicer arietinum
  source 1..1937: mol_type = genomic DNA
gagtatcatagacgagacatgatcaatgtaagttgtttcactttgaaataaatttgaatttaaatgagtttaactcattcttgagtctaaatatcacggaaacggtcctctcttttttttttgttacaaaaaagttcaatgtgacttgaaaagggtggtatcaaagacgattttttaactatgcataaattatattggatatttcattattttgtgaagcaaaattaaaagtaaaataatagttttaaagataactgattttaaatttataaataatgttacatatgaattaatttgttgaaacattgtgttttgtccaatgtaagtttcaattgtttatcaaataacttcatcaaaaaatcaattattaattattaaaaaaatcaaatatgtaaattaaaacattttttttttctttctaatttgaggacataagttatatatattgattcaaagaataccaaatatgtaatgaaaatttaatacaataaaagaaaactgcaaatactttaattatttcttataaatttagtttttcagattcaaattatgtaactttaaaatgtgtggatattgcatcgattctaagttttttgtctttagtgtccaatacaatgtctcaaaacacaatatatataggtgttttgcttagtttgtgtggacaatttatatttttaaatgatcattgaaacatttaagatgaaataatagtaggatattttgttaccaacttcttgtgcagagatttgtaaagttagaaatgaaggagtagccttcctaaaaaaaaaaaaaagaggagtcatgaaaattgattagaatgggatgatggtagaatatttttatgaggaatttgtgtatatttttaacatatatatgatgtgtgaaatgtaaaaatttaattgaaaaatagaagatattgaataaatttatagagacatatgaatggttctacataaaaatataagtaatgtgatacaaatattaaaaagtgacatgtatatgtgcgaagtatcatttaatagagttttaatattgttctaatatattataaatagattactttttttagtagctaattgacgggggtagattattttagcataattatgaattctttgaagttaagcttgtcattttgcaacaaggaactagttgctatagctgctaattttcctcatctcttgtcaaatgtgtgactggcatataataatatgggaagactgcatttttggtcagttatgtcttaacttatttaattactttccaaccgagaagaaaaaacatatttatttacttttttttcttttaataaatagaatagtataattatcgctacacattcacacacataattgcataatcttgaatttaaattttaaaaatattataaaagaaattttttagaacaaactgcacatgcaaatattttaagtgatcactctctagatagtaaacataggaataaaaaaaactgaaattgaaaaaaatagtaaattttaaagtaaaattaatttcaaacagttgatcaagtggaaggaaaaagaatcttatataaaaaaaaaatataatttttatttttaaagtacaagcaaactgaagcttagaggaaaaataagaaatgttatcataaaaataattaaaagaaaaaaattagtaattcaaaattgaaatatgtgagttgattctattttattagatagaagacctctgttccaattccatctacatatgcttccatcatgtgtcattatagacactgtttgattctaatacggcactcacgttgtagccttgacattaattttgtcatccacaagcatatcatatatgttaagatcacacataattttttgatatataaatatccaaggtgatcttaggaaccaacacaccaacttcattcttacatttcactaagcactcaagaaattgttttc

SEQ ID NO: 17 - DNA, 1782 bp, Lupinus albus
  source 1..1782: mol_type = genomic DNA
ccaagtagattgaaatgccctaaactacaagcctatttttttttatttttttttatgtttaaaagaaataaaaatatgcatacagggctgtagttacagtttgggccttgtcctgtggccccttgatagagcagataattgagtgaattttcctactcatattgcatttttaataacccctctacttcattttgaataaaaaattcttccttcttttgtaatcataattttcatatcaaaattcagttacaaataattagaaaatgatttactaacatataaaacctcatttacatccaccaacccatgattccaaggatggtataaactaaaatattatcaagtgttggatgttttttcactcagaataacaaatggtattgtctggtaaggaaataatcacttttagaatgtatatttgttcttcttaacaaatattgttctcatgaatcaaatacttaatacatttaaaaatctattgcaaacttgcaccattaatcaacttgttattggtaaagttggatggttacatggttttaaaagttagtaaaactatataatacatataatgtgacaatgaaagagtatggtacttggtgtggcaactttaacgccagtccttcattacaaatttgtgaatcttcttaacacgcaagagtaataattatgtgagttgcggttttttgttcttttatattccctgattggcaagaaactcctgcatttcacgaatcctttcagtttcatgtcaattccaaaacccatccagagagagaagaagaagaagaaggtaaaggataaggtaattttattcaatgtaaaaaagtgagttttacaccatcaaccatactcttgttgggtttcaaagggtgatagaaatgatagctggatataggaggagattatgttagatgaagagctagaacatacatttgaggagattgtgttagataaactaaacatatatttgattgatcaatagttggagtagtactttggtgggttgaaaggcctagattttcatatatagtagaacacccttcattatcaaactttacaatgaacctaagtcatatttataggaccccttaagaatctcataaaacttctagacaatacatgttatatctatcttaatataatttatctaaaagtttctagactcaagctaaatattatgcattgccacattttttatagaagtctctagaaacaaagctaaatatatcacaataatatagaattctctacaaatatggcatatttggcaaatcccttaagtgacttaaccaactgaaagataaaaacaaaagcaacactaaataggcttaacagaaacagacccaatatgaaacaaagaaaataaaataaaacgaggccctgtggccttataactaaacaacaaaaagtggtgaatgtgtggaaccatgttgcagtaagcaattaacatttcatgtagcaaatatataaaaagtatgtataattagtttattgaaattatttagtagccatactctaattttgtaaccctggtacatccaagatatatatattaaatagactatattttcacccacataattaaggcatgagattgtatgctttgatgtaaaggcaactgaaagaacctacttaaagactactcagtataccatatatgttataacaaatccaacaaactttacttcaaagcaagattaaaacataacctatatataccactctctttgttcattcttcctcacctctttccttctatttacaattcaattaacaatcact

SEQ ID NO: 18 - DNA, 2043 bp, Lotus japonicus
  source 1..2043: mol_type = genomic DNA
gggctgagagcctgagacactggtgttaatgttggtgagggatagagttgggaaggtgggtttaaagcggtggtgttgcaacatgttaggactggtagaaaaagagggtttagactttagagttgattcatgttgcaacaacaatgatatatacacacacattattttcaaaacacttcattttcactcatttttatttttatatctctcttcttatcatttatcacatctcatattttctctctcttactttttttttcttcatatctctctcctgcttcacctctctccacctcatcttagatggtgtgaacataatattattccgtgttgcaacacttaaattggtcaaaataatattttacttttaaaataatgtattgcaatatatttatgctttaaatcattttcatctagttatttatgattgggttaattatgtttcatacgtaaaccacattatagtgtggcgacatcgttcgacacgatgagtttatagcaattaaataaactctataatttacagcagttaaaactcccacagtctgcaagtgtagatagtttctgccacaaaattgtgtcaatgcatcatttttatgtatatttagtttacggcgagtcacggccaaccaaactaattggcacacacaaattgtacaatctgcaaagtatataaaaaacactttgcttacaacttgggagtaattagaacatctacccacttataatattggtttttttttctctctctttagtaggagtttttctacattttatgtaggtttttgctttcattttaccttattttttcctctggccatcacggcctatccttccctgtctacatcttttctttctttcagctgagtttttttttctccccatcttcctcttaaaccctcaattggattttgacctcaatgtatctacgtaaaacttcaacccagtacggttgagctgcttgttctatgattgactcctcgcaacctctgacacaaccaactaacccaaaaataataaggacaacgaagaaaaaacaaataatgtgattcaagaatttttttagtcaagcctagttttttttgtaaaatctaaggagttttttcttttatgatttatttgcgtgttttctttttctttttcattgggctagaaaaaacaaataatgctatgctagtgttttattttgtagcatttttgtattgctctgttatatgagatttgtcctctatttatttacttgatgcaagttctatgtggctgtcccaagtggctttaatctacgcatcattaaaaaaaaattcttcatctggatatgaaatttattgcctaaaagtaatggctttgaattacaagtttaatgtgattcaagttgggcaaatttccccatgactcgtagatctattttaagatacttttcgactctgaatggtgggtgcctatatctatctcttagaaactaaatacagagctatagatgatgctgctagtgagttgatttggctacatgaagtcatgaactcttaaattccccacaagtattctactccagctcttttgtggcaactccagtaggagcattaacatatttcatttacattagtattatcggacctgatgtatatatcataggagtactagatcatgggaccttttttttctgaaatcatcatgggacctcaatgctactagatttgatttatgaaatttgatttacattagaggcatatctaatgtcatatttacataaattttgggccgtttcttcttgtgcaccgaaataaatatactagtattaattaaatcaagcactatatctatcttgagctgcataattaaggcatgtgattttatgctttggtgtaaagctacctgaaagaacatacttaaagactactcagtagaataccaatcccaacaaacttaacttgaaagtaacatccaaaacataacctataaatactactccctctccccattcttcctcaccccttctcacattaattcttctcttctattcacaatccaataacaacaattgcc

SEQ ID NO: 19 - DNA, 1943 bp, Phaseolus acutifolius
  source 1..1943: mol_type = genomic DNA
gacatgtaaccgccatgactcttattatttctatatccttgattttgattaatgatagaaccaatttatgtaaggctttttaaatgcgcatttatgtaattattaaatcatgaaagtattataactcatatgagtcaagtgggagaatgtaagaaattattaaattaatttctataaagtgactcacatgacttagagtttggactttgggcccgaatatgatttaataataataacaacagaattaaaggtccattggttaatgtgagaaatatatatttgatgacatacctaatggaaaacctacatctgatggagattgggaactaaagtatatagggttctgtctctgcaaataatagttgtctcactgttaaccatcacaaaagtgtgtgacaagaacgggattgctaagagatagattgggacaaaggagatagactaactgctcaagtagatcactcatggatcaaggtaagtgcctattcctatttgaatgattgaatgtgtgagaatcatgatatgataagatctgtatgttgtctgtatgagaacatgtattgtatgcgttcataatcatgtttatcgtgtattaacttacagctccaaccctactttatgtatacactaaagatccaactataccacagtgcctggaggtgtaagcgcatacatcaattttgcataaccaagaaaaaagaaacagcatagagtttataacaggaataaaagctttcagagcatgctccctactcatataagaatttccaggaagcaaaagaggtaccaaagcaccatgggttgtcatccccaattagattgttttgtaacaaactatatgcagacttaagagtattttttttttgtcatcctctttcaacaatcatttatctaattgacaactttgaaatcaaatttccctaagttgctgacttaattcttcaagaagagatttctcacattcaaatcattctcttctctagctgaagatccaataccactgatttaattgtcagtatccaagttcccctttgttagtcttttttatgtatatatatccttgttcagaatgatgtaacattattgaagtatactactattattgacatagttctctttacataacagctttattaacatgatgtgtcatcgtgaaagtcgagaccatgaacttgaattgtagactcttaagaatttattttgcataaatattaaaaactaacgtgcttgatagttttaagtacttattttgataatgacctaatttttattttaattatatatatatatatatgtatatatgtatgattttcactttattaaattacactcgctgttactgcattagcatatatgtccgtaaatatctgtttgaaattgaaaatcattggtagtgtaaattttaggggcataaacaaggactactcttttctttcaactttattgtggaaaattttaaatacagttgtcgttctttcaaagaaaatctcaaccaaataatccttctaacacgttatttttcacctttcaccagaaagatacatatttttttatttatatacgtgttctgtcaatgtttgaagtctttttcttgtattaatctagtggacagaaacataagtaaaataaatatacagtatgtgaatattttcagcttggtttaaataagtatatgaaagttctcctactgcaccgaaataaatattctatgatatccactatcttctgccacacaattacggcatgtaatatattattatgctttgatgtaaagccacgttaaaagaacttgcatgcttgaagcctactactcatatcatggtataacaattccaacatctaaaaccccacacaacctatatatataatgctctctttctccattcttcttcatcccttacctattcacttctctctagtattacatttcaacaaccatagcc

SEQ ID NO: 20 - DNA, 1752 bp, Phaseolus acutifolius
  source 1..1752: mol_type = genomic DNA
ccagcatgttaaggagaccagaggataccacagaaatatctgctgcacgagtataaatttaaatgtccttgagatctcttcaaatcaacttcataattttgttttatataattgatcaaaattcctactgtataagattttttttcaaacatattttctcataaatatttagtgtctctatttctatagtaagttgtacttagtaccaattaatatactattgcattaactatcttcattattttttatctcggtactaaacactacaagaaaatcattaaataaaaaccaattttagagacaaaaaataattagttgctatagtgactaaattaaaaacaattctagaaattaaaataattttttatttctattatctttaaatagtttctaaattgatatctaattatctatcaaaattttaactaccaattatttatattttaaatttggtagcaaaaatcttagtagctaattagatacaaatttaaaaactatttaacaataatagaaaccaatttaaaaatcaaaaaaaattttattctccaaaataatatctaatttagttaatataacaactaattatttttgtctataaaattaatttttatggtaccattctttccattttatcatgtcaagttcaaaagagtaaattggtaattatgaatattatgtagattaattattttacaaaccagttaaaagaatattagaataacatgcttttatatatatatatatatatatatatatatatatatatatatatatatatatatataaattgaaatatccttaatatgtcttcaaatatgagataaccttaattacattcaaaagatgtctctcatgtaatgtgtattttcccccattgtatttaacttgtaattattattaaataatatacaaccacttcatctatcttcattattttctattttgcttcgtaattgggtgaatttataatttcacacaaaggtacatttaaagtattactttaatatcacggcaacttttgcatatatagagaataaagtttctttgaaaccaaaatagtaaaaataatttttgaaataaaatattatattataatataaaataataatttatttaagtgtaaatgagtgtaatatatatatatatatatatatatatatatatatatatatataatatgcaaactgatcgtgatcttttgtataattatgacaaacactatcactaaggtgaatgtgaatttgtaagcaagttattgatctcagttgcacacataaagagatagaacgcaaaactcctaattattatgtgaagaagaataaatataaatcatgtttaaatagttaatattaaaaataattgaaaactatttttgttaattaatttttaatattaaccatttacattgtatttaatattatattttttacatattcttggtgtataaaaatcattgtacatgtttataaattctgaaggccataaacagtgacaaatgttttaaaatgaaattgaaaaaatatatatatgaactttctcctactcctatcttagtattatgctttgatgtaaaggcacctacaaaaatatacttgaagactactactactcactctgccctgctataacactcccaacagactttacttcaaagtatcatctaaaactgcacaacctatatatactcctctcttctcccattcttcttcatccctcccctcttattctactcacacttcaacaactatagcc

SEQ ID NO: 21 - DNA, 1843 bp, Phaseolus lunatus
  source 1..1843: mol_type = genomic DNA
cttgacctatgtatactcttacaccaatcacatttttgtttgatttttttaatattaagcactcgagggatttaatttattacaaaaaccatagcacagaaatatctgctgcacgaatataaatttaaatgtccttgagatctcttcaaatcaactttataattttgttttatgtaaaggataaaaattcctactgtgtaagatcttatttcaaagatattttctcataaatatttagtgtctctacttctgtattaggttgtacttagtaccaattcatacacaattgctttaactatcttcattatttttttatctccattggcaccaacaaatgtcctcaagaattcataaagatgaaaaatggcagcaagtgtattccatcttttaatatccttaatatgtcttcaaatatgagataaaccttaattacattcaaaagaagtttctcatgtttttttttaataaaaaataaacaataaaaatattttaggaatgtttctcctataatcagtgtattttccctcactgtatttaacttgtattgttattaaacaatagacacccacttaagttatctttattattattatttaataattgtacttaggtattttgtgtgtaaagtgggatcatcctttaaatattctaaataattcatttattattagttgataacttttttcattattttttatcttaccttctaattgggtaaattttacataaaatacattaaaaatattactttaatatcatgacaacttttgtatatataaagaataaattttatttgatactcaaaataataaaaacaactattttataattatattttaaataaaatattagattataatataaaataataatttatttaaaaatgaatgtaaccggtattaagttacatatattttagttactaatctcaacgatactgaaacgtatatatcactaaggttttttttattttattttatcagcaccactatcactaaggtgaatttgtaagcaagttattgatctcaattcgctcacacataaagagatagaagcaaaactcctaattaatatgtgaagaggaataaatataaatcatgtttaagtacttaatattaaaaataattgaacactatttttgttaattaatttttaatattaaccatttgcattaatgattgtatttaatatatatatattttttacatattcttggtgtataaaaatcattctacatgtttataaaattcgaaagtcataaataatttttttttatcaacaagaaataattaaattaaaacggtccacttaggggtggttcaacccttatatagattaagaaagaaaaaaaaaatatctttccatatacacctctaaatcccataaagaaatctcatattctcttctccaacactactagctaaaaaagaaacgttaaatataatacatacataccaaatattctagataccaatcagagaacgagaaactcaatgacaatatatttaaaacgatccaagattaaaccttaagttgtgcagttaagtataatcaaccgcagtttctttaaataaacaaattattttaaaatgaaattgaataaaatgatatatgtgaaagttctgctactcatatcttattattatgctttgatgtaaaggcaactacaaaaatatacttgaagactactactactgctcactgtgccctgcaataacacttccaacagactttacttcaaagtatcatctaaaagactgcacaacctatatatactcctctcttctcccattcttcttcatcccttccctcttgttctactcacacttcaacaactatagcc

SEQ ID NO: 22 - DNA, 1962 bp, Phaseolus vulgaris
  source 1..1962: mol_type = genomic DNA
ctagatccttacaattcatgttaatcgatggtctaattatttttaacaacaataataaataaattgaattaaagtgaaagagtatgtgttctccaaccctacttgatgtatacactaaagatccaacaaaaattaggtctcacttagaccccccaaaaaattatataaccataaatctcagtccagtcatagcagatcacacatgatacatcaattttgcataaccaagaaaaaagaaacagcatagaatttagaacaggaatagaagctttcagagcatgctccctactcatataagaatttccaggaagcaaaagaggtaccaaagcaccatgggttgtcatctccaattagattgttttgtaacaaactaaatgcagacttacgagtatatcttttttgtcatcctctttcagcaatcatttatctaattgacaactttgaaatcaaatttccctaagttgctgacttaattcttcaataagagatttctcacattcaaatcattctcttctttagttaaaagattgatttaattgttagcatccctattgttaattggtctcgaatgagtttggggcctagaaaacgattgttcctgcttccaccttcggtcggtctacgatgctcccagtcctattacaccttcctcctctcggtcgtctggctccacaagtggggttggtacctgcagaagacactctaacgctcaagtcagtcggggtgttagcactcgggtgtcagagtaccataaataatgacatacctttgtccttgaaatgcgtgttatttatattaccttaatgggtcttcattgatgggccgaattagggaattggttcgtgtttagagttgcgctaaacaatcattaactacggattaaccctgctgacttggtcaaccattgactaaacgttaagggtcagtcggccacgtgaccgcgtcgtcatttcttaggcagtgacttagctcgagtactgaccgactcactggatcgtccagtcgtaccactacattagtttttttatctatatatatccttgttcagaatgatgtaacattattgaagtatactattattattgacatagttctctttacataacagttttattaacactacagtatattaacatgatgtatcatcgtgaaactcgagatcatgaacttgaattgtagactcttaagaatttattttgcataaatattaaaaactaacgtgcttgatagttttaagtacttgttttgataatgacctaacttttattttaattatatatatatatatatatatatatatgattttcactttattaaattacactcgttgttactgcattagcatatacatatatgtccgtaaatatctgtttgaaattgaaaatcattggtagtgtaaattttaggggcataaacaaggactactcttttctttcaactttattgtggaaaattttaaatacagttgtcgttcttttaaagaaaaatctcaaccaagtaatccttctaacacgttatttttcacctttcaccagaaagatacatattttttttttatttatatacgtgttctgtcaatgtttgaagtctttttcttgtattaatctagtggacagaaacataagtaaaatatacagtatgtgaatattttcagcttggtttaagtccattaattcaccttaagtaaatgaaagttctcctactgcaccgaaataaatattctatgatatccactatcttctgccacacaattacggcatgtaatagattattatgctttgatgtaaagccacgttaaaagaacttattgcatgcttgaagcctactactcatatcatggtataacaattccaacatctaaaagcccacacaacctatatatataatgctctctttctccattcctcttcatcccttacctattcacttctctctagtattcacatttcaacaactttagcc

SEQ ID NO: 23 - DNA, 1839 bp, Phaseolus vulgaris
  source 1..1839: mol_type = genomic DNA
ggtatctaattagcaaccaaggtttttgctaccaaatttagaatataaataattggtagttaaaaccttggtaactaattagatatcaatttagaaactaatttagaaagcaaaatttcttttagtttctaaaatatctctagtttagtcactataacaactaattatatttgatttctaaaattagttcttatttcatgattttcttatagtattagtaatctcacatcgttaactcacttaatcatctttcaatgacaaaaataataaatttatcagcaaataagggtttgtcttttaaacttatttggatttccataaataatatcaatgactttaagtccattaaaataagattattaagatataatatatgtcaacttatttttaaagatagaatttcctaaaaattcttctatatatactaagtttgatgtgacatatactagaaaaaagcaccaaatttagataaaaaaagactaaaccttgttttttctaagacatcttagtatactatgtcaatctgtttatataaataatggtatataattcatgtatacataataacataaaaatatgtaattacttatacatggttttagactaataaacctatatatatatatatatatatatatatatatagaaatatagaaatataaatgtgttaattaatattttatacttttaaaataaaatagagttatactttaagtaatttttttgtaagcatgaaacctttaaatagacttagtaccaattaatacacaattgctttatcttcattatttttaatctcggtactagaacctcaagcttacgctctggtaccataaattcaacctaaaaatgctctttccattttatcctgtcaagttaaacagagtaaattggtgattatgaatattatgtacattaattgttttacaaaccagttagaatattagaataacatgctcatatatatatatcaacaacataaatttaaatttaaatatccttaatatgtcttcaaatatgagataaccttaattacattcaaaagatgtctcaaatgtaaccacgtgtattttcccccattctatttaacttgtaattattattaaataatatacaaccacttcatctatcttcattattttctatcttgcttcgtaactgggtaaatttataatttcacacaaaggttcatttaaagtattactttaatatcatggcaacttttgtatatatataaagaataaagtttctttgataacaaaatagtaaaaataatttttgaaataaaatattatattataatataaaataataatttatttaagtgtaaatgaatgtaaccgtgtattaaagaacacttattttagttactaatttcaacgaaacgtgtatatatatatatatatatatatatatatatatatatatatatatatataatatgctgatcttgctcttttgtataattatgtcaaacactatctctaaggtgaatttgtaagcaagttattgatctcagtcgcacacataaagagatagaacgcaaaactcctaattattatgtgaagaagaataaaaataaatcatgtttaaatagttaatattaaaaatatatgtgaacgttctcctaaaccctatcttagtattatgctttgatgtaaaggcacctacaaaaatatacttgaagactactactactcactctgccctgctacaacactcaacagactttacttcaaagtatcatctaaaactgcacaacctatatatactcctctcttctcccattcttcttcatcccttccctcttattctactcactcttcaacaactatagcc

SEQ ID NO: 24 - DNA, 1944 bp, Trifolium pratense
  source 1..1944: mol_type = genomic DNA
ggacggattgggcctcattctttaattttgcggtttagactaggtaacaattttggacccgaagccgctcagacccgctcatttccacccctactctttcctcttttattttcatttcatttaatttaaaatctgcacataatattactagatctctcttgtcttatactaccatacaattctttttcaaattcattaaataacacatgttagaccaaatatttttactgaattcacatatcatgctgttgtacagtatataataattttcaagtcatgtttgacattttgtttaaaaaaaacattttttttttcgatttttgtacattaactctttgtaacctttgctagtctctcttttagcacaccttgtgagaaagtgtgaaaactagagattccacgtaactattgtacactaagttgtagcaatgagacattacaacgatttgagtgattttacttgagagttaaaatcatgtattactccatttattttcgccattaataaaaactacatcaattttcatacacaaatgaagaaaaaaaattattgattttataaaaaattatatcaccgaagtctaacacaaatggttaatacgaatggtatgtcggtgttgtaattttttagaattgagtttgacttttactaataaaaatttcaaaaccactttatttttaaaagttgtttttttttttatttggtacaattaaaagctgtcttttattaacttcatctataaataaagtttcaaaacaacatacttcggttgggcatgaatttccaactaaagaaaataacttttttttttaataaaaagaaaaaaacttgatattaagtgatgtcttggcagtttgagactctttgtatttgatattttaaaaacaaaatcggaagtatatttttcaaaatttaaaatataaacattttttgtgaaaaaatataggcacccatttctgaaacaatttagattccaaaaaaatattcatggtggggattggcaccggacaccctccgatgaaattatcacggaaaaggatccatgcccataagttgagtaaggtgtcaaacttagcatgtttttacacattcgataactctttgtagaagatgaactaaatcaatttgattgctgaataatcttatatcttatattatgttaaggatcatgtctacaagactacaaataatttagacactcatcaatcttatagagtaagtattttatcaaactaatcatccttgctgcaaattttctttattttttttcaaaataagcatttaccttgatggtgtcaacattgtttgttcactttaatttttaactatagcaaaaacaaaattccctagacaaactataaaaatatttccccctaaattattcaacattgtttgatggctttgcatcacatcacattgtacagtgcagttgatgcattggatccaaacacgacatggacgaggtttaagcttaaaatgggaaatcatcagaataagaaaaagattattgtaaaattatgggtgtgtaaatatatttaacaagggccaaataaatagtctgctgtttctttaggcccaactattttttgaccaaacatgagatcaatgatatatttgtttaggatatgtaatgaagcttacaatggatgatcttgcacacaaattctcaattaccgttgctgtaattttggttctagaattgtttgcgtgcttgttctccaattggatataaagaacatgcttaaagttaaagactactcactataccatatcataatacccataccatagtataaaataaatatttcaacatactttactttaaagcaatatctaaaacacaacctatatatactactcccttaccacattatttttcatcccttctcttatattcttctcttctatttaccaattcaattacacgttatttgtttccaatc

SEQ ID NO: 25 - DNA, 1991 bp, Trifolium pratense
  source 1..1991: mol_type = genomic DNA
ggacttgtgttatgggcgatgtctgagatggtaaagaatccaatggtgatgaaagaagcacgagctgaggtaagaagagtgtttgaaagaaaggggtatgtggatgagacaaggttgtgccatttgatatactgaaagggaatcgaaaaaattgaatggaagaatcgaacaaaagggaatgagtcgaactattttgtgaatcaaagatgcactgctgattttgtgaatgagactggaaaatagacttaatttctgtcactgtgaacaagatgattttattatgaatcaaagactccatctttctttttgatttttttttttttttacaaatctttctttttgttacaaatttgttacaaatcttttttttcatcgttttcctagttttttgtttttttttgttgtttttttttatcccttaaatttatttatttattttaatttttattttcaaaataaaaaagatttatatttatatatttaaaaaagatttttaaatatatgtttactattttattcctatttttaagcaaatcatcttttttttatatctttgtatatttttttggtatcttttctatattttcttagtaaaattacaatgaaggatataaatcgtggaacatgattaaacgtaataaggataaattcggtagtttggtaatatgattttaatatattagaagtagaagtttgttaggatttgcttaacttttgggagcaataaagattttgttatgacttgtagaccaagtaaaacacctattagatggtcataaataggtgatcaactttgaaaattaggattcacaccactctttcacataaatcagaaaataaaaacctagagatccacgatcaacaagatgttggaactccgtctagttcaaggttcacttctcaagaaagttcttgaatcgatcaaggaattggttaccgatgctaactttgattgttcttccaccggtttctcccttcaagccatggattctagccatgtcgctttggttgctcttttgcttcgttctgaagggtttgaacattatcgttgtgaccataacctttccatgggcatgaatattaacaatatggctaaaatgttgaaatttgccaaaaatgacgatatcatcaccattaagactgacgatgggagtgatactgtcactttcatgtttgaaagccctacccaagacaagatttctgattttgagatgaagctgatggacattgatagtgaacaccttgtaattccggaggctgaatatgatgccattgttaggatgccatcggctgagtttgctaggatttgcaaagatctaagtagcattggtgacactgttgtcatctcgattactagggaaggtgtaaagttttcaaccaagggagatattggaagtgcaaatatagtttgcagacagaatactactgtggatgatgctactgtcatagagatgaatgagtcggtggccttgcaatttgcattaagatacatgaattcttttacaaaggcgactcctttgtccaatacagttaccatcagtttgtcaaatgagcggccagttgttgttgagtacaagattgctgagatgggatatgttcggttctatttggctcccaaaattgaggaggatgaaccagagacaagtttcgtttgataaagatgtttgattgtcgttttaagtttaatttgatataaagatgcttctatacattgttaaggtgttggttcagtattagtataattgatttgatttgattattattttatttaatttgctttaatcaagagttttgattaaattaattttcatatatatttaataattggaagatagtcaaagatttattttaaaagagtttccttaagtttagattaaaaaaataaaaaacagttgataaatttaaaatatggttcaatggagttctaattacaatttgacttacttaataaaccttgcaagtaagtaaaaccgcttcttaattcgattatt

SEQ ID NO: 26 - DNA, 1995 bp, Vigna unguiculata
  source 1..1995: mol_type = genomic DNA
cgatggacattgcggaagtgacctagatcatggtgtagcagctgttggatacggaagtgcgaaaggtgtggactatatcatagtgaagaattcatggggttctaagtggggagaaaaaggatacatcagaatgaagaggaacattggaaaacccgagggaatctgtggaatctacaagatggcttcttatcccactaagaacaaataagattgattactccattgttgtgtctcaatttctttccttcattctacctgtttcagcttttccttgaaaacaatgtaacttttcccacataatgataataaaaatttcgttaactcttctcattattcccttgagtcttgagggatttttccttcttcgttgttctatgtttcctttccaaaaccgaaaggtatagaactgagaccactgttatcactctaatttaaagtagtgcaatgagaacacaaatgtatatatatgatcaccatgacaagaacacagcacacctttcatttagggttgtcttaaaagaaataatctaatacactataaaagaaaaaatataacactattctgcattgcgaaatttttatgcacccaaaattttaacgcgatatcgtatcacatcctaaaagagttatctattactctgtctaccctgataatatggtatcaataacatgagtatgtcaaaaaacgttggtttaacatttatcaaattatttaattaaaaaaaaaacatagcttttttttatttttaagaagcttctggtaattgatattcaaaacataaataagtataaaaaaaataccaatgaggtgaattagtaattagataaaaacaaagaaaaaaaatcatgaatttttctaaaataaacaacttgaaaaaataaaattgatttagtaagaaaaatactaataattagcatttgtttataaaataaaatcagtaacaaaaagtatgaaaaagaatccatagactttttattatacctatatttctttacaaatctgaaagttaactttttttaatgattaaaatacttctaaaaattagttgtcatgggtgcaaagtggtgtaaaaaaatattttcctaagaaaaaagtttcaatctcattctgcctcaactctcttgaatcacccgagtgcagatattattatctttcaacttctttatggaaaatggaaaatttcaaatacagtttctgttctttcaaagagaaatctcaaccaaagtaatccttctaacacgttttttaacctttcaccagaaagatatatattatttagggttaaatatgtttttctccctcaactttaagtgaaaattgaaattagtcccttatcgaaattttggttcaatttaatctctcaactttagacctgcatgaatttaatcctttgaaacaaattttgataagtttatttaacgtttcaaaggcatttcatgatagtatttgagatgtttacacaatttgacacatttttgcttaaatattagctaataaactatcatgaaacacatttaaaacgtcaaataattaaacaaaatctggttaaaaaaactaaatctatacaattttgaagttcagaaactaaattgaaccaaaattttgaagagtgactaattctaattttcactacttaagggacaaataacatattttacactattatttacatatgtcttgtatcaatgtttgaagtctttgtcttgcgttgaaccgaaacataagtaaaatattatccgaataagttctcctactgcatgcaccgaagtaaatatattgtatcatatactccatcttgtgccacacaattacggcatgtgatataatattattatgctttgatgtaaagccacgtcaaaagaacatgcatgcttgaagcatactactcataacaatcccacatctaaaaaccacacaaaacctatatatataagcctctctttcctcattcttcttcatcccttacctatattcacattgcaacaaccatagcc

SEQ ID NO: 27 - DNA, 1927 bp, Vigna unguiculata
  source 1..1927: mol_type = genomic DNA
gcatgcataaacgtatccatctcaaatttatgcttaaaataaaaattaaagaattgcattataatttcaatttcaattttaaattagtaataaatgttgagaatttgttctggaacgtttaaatggatataaaaatttgaaggtgagaaaaatgatgatatacaaaaaaaggaaattatttggtacgggtgtatacaataatgggtaaccgttaggacttaggatatgcaacaagttagcgtatgatctacatgtacgtacgctgtacgtactctgttatcatagcttgggccgtattgacaggaaccatacataataaaagagcaataatatcaaggacaaatactgaagaccgacaaaaacataatgttaagtattgaccacattttaaaaaaataaattatctaatttggaatcacaatatataacaaggttaaatttctcctatattataaaataaattcatcatgtaaaataaaatacacagcttcaacagtgagattatttctcttctcataattttaatttaatatttttctctctttctttacatacatttcacataattaatattccacaataagccaaaatttcctcacataacaatatccaattacactcattttaacgtattttttttttctttatattggaattaaattccaatatagaaactgtagcggcacctacccacacaggatccagatataagaaatcaataaaatggcatacataataaagaaaaattccatgataatgtaatatctcattaaatctcatatatttagtcttaggatatatgccattttttttcagagagaatttcctaaatattaaaaattacttcctagtatatactaaatttggtgtgatgaagtgtttgaaaaatttaagtttcacccacattaggtaaaaataataaaattaaatagatttaaaaatttattctcttaaaatttttggtgaaagtaatgtgaatttttatataagttagactcaatatatatatatatatatatatatatatatatatatatataaacatcacccatgtcttttacaaaatttagataataaaaaacactaaactttatgttttctttttaagaaaatcctatctagtatcatatgtcaatctgttcatataaataattgtgtaatgcatatattatacataataacataaaaatatatgtaattgcttatatatggttttaggtcaagaaacctatatattaggtaaatgcggcaattagtatttttataattttaaggttaaaatagacgtatactttaaataaaatatttgtacgcatcaaacctttaagaagacttataatttaggtcaaacttaaagtcttatctaacttgactatttatactcttaagtaaatcacataattttttgtttgtttgattttttattttagtattgagcactcgaaggatttaatttattagaataaccagcatgttaaagagataagaggatactacaaaatatctgctgcacagataaatttagttttatttaaaatgatccaattgtataagtcttatttgacagataaatattgaatctctgttataataagtttggttggtttttcagttgcaaattagtaacatcattatgagtcctaaaaaaaccataggaagaagcttatgataacctagtttaaaatccaaggttaccataaaccgacctttcacataataaatccaaggttctccaaaacatatcttagtattatgctttgatgtaaaggcagaatcaaaaatatacttgaagactactacaatcccagaagactttacttcaaagtatcatcacacaacctatatatatacttctctcttttcccattcttcttcatcccttccctcttaaattcttctcttctactcacacttcaaaaacatagcc

SEQ ID NO: 28 - DNA, 168 bp, Glycine max
  source 1..168: mol_type = other DNA
atggttatggaggcctcggcatccatttgcaacaacagacccttcatcttctcttcctctcgtgccataacacaaaaatcttgcactctctcatcacctatgcttgtcaaatccatggccactcccaagcctcttccctcggtctctagaactgtaggctctagaaag

SEQ ID NO: 29 - DNA, 1167 bp, Glycine max
  source 1..1167: mol_type = other DNA
gtcagtgcgcacacacacatattctttttgttcaaatggtttgctatgaagtgtgacacttatctattatttctttactactaaaagaatgaaaagggtattgtgaatttaactgttttgaacaaaatattagagaagaaggaaacattgtaatggttattagctcttaaattattgtaggtcagtgtaatatatatctatatatgtagaaaacttgcttacacaaattgttctttttcttcaaatgttttgtgaaactgaaacctgtctatagtgtttgaatttgatcacttggaaaaaaaaaagaaaatgatctgtggtgaattagctgatttgaacaaaaattacagtttaatggttattagtttttaaaatattgcaagtcagtgtaatatctgtctatatatctgtttatgtattaaacttgcttacataatgattctttttgttttattatgaagagtgaaacctgtctccaatgtttgaaacttattattaaaaaaagaaagaaaaatatctgttgtgaattgcctgttctgaaccaaaaaatacattatgaaaaaacaaaattgtaatggttagtattagctcttaatttttgtgagtcagtgtaataatatgtatctatatatatctatacgtattaaacatgcttacataatgattctctctgttcatattatgaagactgaagtctgtttctaatgttttaacttaaccactttaaaaaacaggctcttttgtgaattacctgttataaaccaaaaattacattggaaaagacattgtaacggttgtaagctcttaaatagaacttgttcaagatagtttccaacatatgaaagaacacctcgtttaaatggcataatggcatgagtgcatcaaataactaaatggcaaaacttagatgtagtttcatagttattttttgtgctattttctagtcaaaattagtgttttacctcagtaatcagcgtaataaaggtttgtattaaaggtcaacatacattaccaaagtgtaagtccaactctgatcggaaaacaacatcaaaaacacctaagaaactgtgtgcagtttttgtcctaaatgaaaaattgtttttcttttcatctggttaagtagtagtgtttagttacaataataactgaaccaagtgcacaatgcag

SEQ ID NO: 30 - DNA, 168 bp, Pisum sativum
  source 1..168: mol_type = other DNA
atgatgatgacaatgaaaacctcaccatccatttgtagtaacagcttcatcttctactcttctccatcaacacaaaactctcacactttctcacctttccttgtcaaatccatggctactcccaaacctcctccaccatctgtctctagaaccgtagcttctaaaaag

SEQ ID NO: 31 - DNA, 581 bp, Pisum sativum
  source 1..581: mol_type = other DNA
gtcagtgcaaaataatcactgcataatcaatatattttccttctacttgttattatgatttgttataatttatatagtttcttaaattttgattccttatatgcaatattatgaggagtgcaactgtggaacatgtggaacataaattgttattgatatgtaagaaaaagattggtataaaacatttctactctaagtagaatttaacataagcatttgaaatgatattgcacggattcgaatccgcattttagagccgtctaaagtttttagagatacaggcttgttaactatggtttatggtttaaccctgacaagacaattgaagatttttttttagttctttagagaaaataattaataatgagtagaaactcatacatacaatcgtgactatcagaattcaaagtctggtgattgcgtataacctatcaatatcagcttttttctgttatgtcagagcccgacaattttaagactatatgctttaatccacctgcacacttttaaagacggccctcttgttttcggtgtcaaaattttgatttttaactgttgctaattcactaacacaattgcag

SEQ ID NO: 32 - DNA, 150 bp, Medicago truncatula
  source 1..150: mol_type = other DNA
atgatgatgacaatgaaggcctcagcatccatttgtagtaacagcttgatcttctcttctcctccctcaacacaaaactctcacactttctcaccttttctggtcaaatccatggctactcccaaaccagttactgtcacttctaaaaag

SEQ ID NO: 33 - DNA, 1064 bp, Medicago truncatula
  source 1..1064: mol_type = other DNA
gtcagtgtaaaacaataactatattattacttgttattatgatttttgatgatttacataattgcttttattccttttatgcaaaatattatgctgtgtggaactggaacatgtctataatattatattatttaccactttaattatgagaaaggttgaaaaactagctcttataatattataaaacgtatgtgaaatttaacaatatgtgacatcggttgttatgggagttaattgatctttaagaaacaaacttgtataaaacttttgtcgcgtaagtacaactcatatggtaaaagactgaggagacatttgattttctcagaggcgggtttgaacttccaattccctacttatcaacacttcgtgtgtgtgtgagttttgtcgcttgctggactatttagggaaaataaaggaactatactgtcaaaaaatttcaataatttaaccattagcctacgtagaaattttactttgtcaggcttttaagtaggctaacacgccaggccttcgaaaaggtcagacaagtactaaaaatggtgtatgatagtgaatttatcacgtttagaatttaaattttttaacagaccaggcatatttaaacaaagcatttctaaatatggcattatgtaaaacatgttttttgagaaaattctatggtggtccagttagataggtgtccaaggtggattaacacttgaaaataacccgaaactatgactcatggtacttgtcggaagtagaaactcatgatcctctttgtcttcatgagtgagacggtgacttgattgatactagttcgaaaaaaacatatgggaaaacgaatgagccaacaaaactcaactcatattcgtttttctatgttttggtagaacacgcaccaatcaagtcaccatatcacctatgacagcaacgatgactgtgattttctacttccaagatcaacatcggttgtcattggtatctctacgataattttcaagtggtctaccgtagaaccagcctgttttttggggagaaaagttttatttcttacagtaactaagtcactagcacaattgcag

SEQ ID NO: 34 - DNA, 377 bp, Glycine max
  source 1..377: mol_type = other DNA
atggtttatgggatactattctttgtcctatttgcattcactttgagtttagcttttctactctcaaagtgtctatcaaaaagtcaaacaaaaaatgttccaaaagggtctctggggtaccctatcatcggagagacacttagttttctcaaagcacagagacaagataagggttctgtttggttggaagaacggatatctaagtatggccctatcttcaaaacctccttaatggggttccctacagtctttgtaattggacaagaaggaaacaagtttgtccttggttcaccagatgatttgctttcttctaagaaacccctcaccctgcgaaagatacttggaaggcaaagcttagttgaacttacaggaccaag

SEQ ID NO: 35 - DNA, 626 bp, Glycine max
  source 1..626: mol_type = other DNA
gtacataattttcaacttaactcactgattcagcttaagttgtctgtttaaatgatttggatgaaatgtcaaaataattcttatatgatgatgatcttgattcaaattatgacactttgaaataaggtgtttaacctctgaatggatggattatggatagttggatagtggataagtgattttttggattagtggtagtatttagataactcattactgttataaagggacttactggtggtccttgtttcaaatttaatagaaatgaatttgaaagagttgtggttaaaaagattaatttgtgttggggaatgatatttataagtgagtttaaaagaattgattttgttttgaatactaaagtaaaatcactttagaagtgtaaatatatattttcaggtctcatacccaaaatcaatttatgtctaaagaaaagttagaaagtttcatcctctcaaaattatttctactcactgcaaaattgattttcaaaatcccaaaaagaaattggcaaaaaataatcaattcctagataatcaaatattatgatctccagtgtgattctcacgtgttacccaacaggatactagtcatctgttttgaggctttgttaatgtttgatgcag

SEQ ID NO: 36 - DNA, 377 bp, Pisum sativum
  source 1..377: mol_type = other DNA
atggttaaggatattcctttcattggtttatgtatactaactttgagtttagcttttgtcttgaaaaaacttctctcaaaaagtcaaacaaaaaatgttccaaaagggtctcttggatatcctattattggagagacacttgagtttcttagagcacagaggcaagacaagggctatgagtggatacaagaaagagtatctaagtatggttctgtctttaaaacctccttaatgggttctccaacagtgtttattattggacaacaaggaaacaagtttgttcttggttcctcggatgatgttatttctgctaagaaacctacaactcttcaaaagatacttggaaagcaaagcatagctgaacttgtaggaacaag

SEQ ID NO: 37 - DNA, 503 bp, Pisum sativum
  source 1..503: mol_type = other DNA
gtacatattcttaaattattacttatgatcttgttatccagccaaaaaatcgattaatcttggagatcagtcgattttttttagagttgttgatttataaatatcataaatttgtttagatatatatataccgataccatattccgtaaccaccatatttttaaaataaagaattcggagagaaagaatctatagaactttggaagcatgagtgaaaactcacaaatggaattatattgttaaaacatgttgaaatgggaatacaagatgtataagaaggttaactaactaattagttctcaacaaattgaaactataactaactaattattccctatttatccatatacttatatgtaacaaatagtctacacatatcacaagaagggtcatatctagttttggacagggactctagttttggacagggacacagacacacctttttttccagaggtgtcgatgttatgtgatttgagctttttgtttattttttcatgcag

SEQ ID NO: 38 - DNA, 377 bp, Medicago truncatula
  source 1..377: mol_type = other DNA
atggttgtggaaatatatttcatagttatatgtctactcactttgagtttagcttttctcctcacgaaatttctctcaaaaagtaaaattaaaaatgttccaaaaggatctatgggatatcctctcattggagagacatatggttttcttaaagcactgaggcaagacaagggctctgagtggttagaagaaagggtagctaaatatggtcctgtcttcaaaacttccatattgggttcccctactgtgtttattattggtcaacagggaaacaagtttgttcttggttcctcacatgatgttctttctagtaagaaacctccaacacttcaaaggatttttggaaaacaaagcctacttgaactcacaggatcaag

SEQ ID NO: 39 - DNA, 665 bp, Medicago truncatula
  source 1..665: mol_type = other DNA
gtacatattcctcattgatgcaacttaaattgttgatttaagtagattagattatttgtcaaaaatccttatatataacgatcaaggttttataaaacgatttgtgattgcaattgtggccgcaaccttaacgatcgagggtttataaaacgatatgtgacaccccctcacactcaggattgcacatctgaagcgtgaacataaatgacgggtggtccgataacggaaacctgataacaggtggtccaaagaaacgcgaagaggctctgataccatattagaatggtggttgggcctaacacaaccccacaaaaccggcctttgaggtgaggattgcccccacttataaacaatttgtcaggccaattcctgaccgatgtgggactcttaacattagttagttacactagttagatagtactctaattagttagttagttacactactagttgtttttctcatttgcaaaaaatatcttatgtatcccttgatgtgatttattttatcaatataattttaaattttacttttctcttatcgatgtcatctatcaagaatagcaatatgatattagaatatgttttattctatcaagaatagaaaaatgaaagatcgcgatgcaactgtgatcataatttaaaacctgtgtttattttttgtgtag

SEQ ID NO: 40 - DNA, 377 bp, Medicago truncatula
  source 1..377: mol_type = other DNA
atggtagaggatatacctttcattgctatatgtgtactcactttgagtttagtttttctcctcaaaaaaattctctcaaaaagtcaaacaaaaaatgttccaaaagggtctttggggtatcctattattggagagacacttaatttccttaaagcacaaaggcaagtcaagggctctgagtggatagaagaaagggtatcaaagtatggtcctgttttcaaaacctccttgctgggttctcctacagtgtttattattggacaacaaggaaacaagtttattcttggttcctcagatgatgttttgtctgctaacaaaccccctaccattcaaaagatacttggaaagcaaactctagctgaacttataggatcaag

SEQ ID NO: 41 - DNA, 1750 bp, Medicago truncatula
  source 1..1750: mol_type = other DNA
gtagatactcttcaactcttaatcattgttgcaacttaaattgttgttttaaaatttcaaatagtttagatagcttgtccaaagtgaaatggtattttaagggggataattaaatggcatcatttaaacaatatttcattgtggttaaaatctcgatcagcattctgaaatcttatgattttgtgattttgctctgccttaccgatcagaatcctgagtagaatcctaaactatgtaaaatccgccatataattggcgcgttcgtgatgtgatgtaggcttgtaggatctagattttgagtatttttttttgacttggttgttttatatattagagatttgggactttgagtcgtttattttaagatgtgaaacttggttgttttatgctaattttaagatttcaaactttacgtggcttatgcacgttatagcaaagttgttaaaatcaggattttgcttcaagtcgaaagggaagagcaaaatcgaaaatcttaaaatcgcaatcaaaatcgaaagattttactcaactacttttgtaaaatcgatataagaaatcgtaaaatcacaatcaaaatcaatggattttaccaaaacaaaaacaatgttaaatatatgttataaattatatatagtcataaatgactcggtttaaatccacaaaatacttatacataaatgactaaaaaatggacaaaatccttaaaaccccttttcaattcctgaatttttgtatggatttcacgattctgtgctattttactaggattttacctattttgggattctgctatggatttgagtcatagaaggtgaataaagtcgcaaaatcctgcattttacgatttaagtcaggattttgacaacaatgctttatagtatatatttaatattatttgattaaaaaccttcgattttggttgccatttttggttttgtgatcttgctatcctctttcaaagttaagcaaaatcttgattttgacaacctttgttgacacaattatgtacagttttgttttatattagttaggaactgtatatcaactgtgtgtgaagcatcttatgtaaatgttttcctatttttaacatacaaatttaacatcaaaattcaacaacaatttagatgcttaacgcaatttaaatatgtggctaattatgattagtagatgattaatgaatatggaagatgctaaaacttattaaacacttactcaacacaaccacacagtattgatcgttattaatcacaaatttcaagtgtatatttgaactcaaatagtggtcagtgagtataatgtatagttgaatatttgagtggaacatgaagttgaaaattgtgttatagtgaatggttaataagttaaaactcctttatttagcatatttaaccgcgagtttgaattgtgctaactatctaagttgatgtggaatattgatgtcaaatttgtatgtcaaaataatctttattataatcaaggtttttaaaaactcttgcatgatccaaattgaagcggcaacatcaagtttttctttattttttatgtcaacacatttgaccaaagttttgcataagatcatgacgcaacagcgacaacagtttcaattttaaaccttgattgtgatcttgattcaaacagtggcgctgtgaaataaggtctttcatatttggacacacaatttgaagaattgatatttataaaaggatggtgaatccatctagtttgaggcttgtgtttactttttgtgcag

SEQ ID NO: 42 - DNA, 842 bp, synthetic construct
  source 1..842: mol_type = other DNA
agatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggact

SEQ ID NO: 43 - DNA, 304 bp, synthetic construct
  source 1..304: mol_type = other DNA
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccgta

SEQ ID NO: 44 - protein, 240 aa, synthetic construct
MDLRLIFGPTCTGKTSTAIRLAQQTGLPVLSLDRVQCCPQLSTGSGRPTVEELKGTTRLYLEDRPLVKGIIAAKQAHERLIGEVYNYEAHGGLILEGGSISLLRCMAQSSYWSTDFRWHIIRHKLADEETFMNAAKARVRQMLRPAVGPSIIQELVHLWNEPRLRPILKEIDGYRYAMLFASQNQITPDMLLQLDPDMEGELIHGIAQEYLIHARRQEQEFPPVSVVAFEGFEGPPFGMC

SEQ ID NO: 45 - protein, 302 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGFYAAGNGGGSAVLLDTSSDWGSSGAAMATETCFLQDYMGVTDTGSSSQWPRFSSSDTIMAAAAARAATTRAPETLPLFPTCGDDGGSGSSSYLPFWGAASTTAGATSSVAIQQQHQLQEQYSFYSNSNSTQLAGTGNQDVSATAAAAAALELSLSSWCSPYPAAGSM

(19) United States
(12) Patent Application Publication
(10) Pub. No.: US 2025/0051792 A1
(43) Pub. Date: Feb. 13, 2025

(54) SPATIO-TEMPORAL PROMOTERS FOR POLYNUCLEOTIDE EXPRESSION IN PLANTS

(71) Applicant: Benson Hill, Inc., St. Louis, MO (US)

(72) Inventors: Ross A. Johnson, Olivette, MO (US); Jaya Soneji, Wildwood, MO (US)

(73) Assignee: Benson Hill, Inc., St. Louis, MO (US)

(21) Appl. No.: 18/719,701

(22) PCT Filed: Dec. 15, 2022
(86) PCT No.: PCT/IB2022/062332

§ 371 Date: Jun. 13, 2024

Related U.S. Application Data
(60) Provisional application No. 63/289,810, filed on Dec. 15, 2021.

(51) Int. Cl. C12N 15/82; C07K 14/415; C12N 9/10

(52) U.S. Cl. CPC C12N 15/8234; C07K 14/415; C12N 9/1085; C12N 15/8262; C12Y 205/01075

(57) ABSTRACT

Provided herein are compositions and methods for expressing a polynucleotide of interest in a plant or plant part. Compositions include promoter molecules that initiate spatio-temporal expression of polynucleotides of interest, and DNA constructs comprising the promoter molecule operably linked to one or more polynucleotides of interest. Plants and plant parts comprising the compositions or produced according to the methods are also provided.

### RELATED APPLICATIONS

This application claims priority to U.S. Provisional Application No. 63/289,810, filed on Dec. 15, 2021, the content of which is incorporated herein by reference in its entirety.

### SEQUENCE LISTING

This application contains a Sequence Listing which is submitted herewith in electronically readable format. The Sequence Listing file was created on Dec. 6, 2022, is named “B88552_1350WO_SL.xml” and its size is 105 kb. The entire contents of the Sequence Listing in the sequencelisting.xml file are incorporated by reference herein.

### FIELD OF THE INVENTION

The present disclosure relates to compositions and methods for expressing a polynucleotide of interest in a plant or plant part.

### BACKGROUND OF THE INVENTION

Expressing a polynucleotide of interest in a plant or plant part is useful for product development. Certain polynucleotides may be desired for limited expression across growth stages and/or tissue types, such as when their expression has negative consequences on plant physiology. For example, while causative nucleic acid molecules in organogenesis or embryogenesis, referred to as “morphogens” herein, can be used to improve plant transformation metrics, their prolonged expression can prevent healthy, fertile plants to be regenerated. Additionally, there are molecules, such as transposases, that may be desired for regulated expression in plants, but their constitutive expression may have potentially fitness-compromising impacts. If expressed in a spatio-temporally specific manner, it is possible that any toxicity of such molecules can be avoided. Accordingly, optimization of upstream regulatory elements for a polynucleotide of interest to enable its expression in a spatio-temporally regulated matter in plants or plant parts could offer important commercial advantages by, for example, enabling regeneration of healthy, fertile plants whilst enhancing plant transformation after delivery of a transgene or a polynucleotide of interest.

### SUMMARY OF THE INVENTION

Compositions and methods for regulating gene expression in a plant or plant part are provided. Compositions can include nucleic acid molecules comprising a promoter molecule for expressing a polynucleotide of interest, or DNA constructs comprising the promoters operably linked to polynucleotides of interest. The promoters can enable desired spatio-temporal expression patterns of one or more polynucleotides of interest. Methods of expressing a polynucleotide of interest in a plant or plant part and methods of transforming a plant or plant part are also described. Plants and plant parts comprising the compositions or being regenerated according to the methods of the present disclosure are also described.

In some aspects, the present disclosure provides DNA constructs comprising, in operable linkage: (a) a first promoter molecule comprising a nucleic acid sequence that shares at least 80% sequence identity with any one of SEQ ID NOs: 1-27 and retains transcription initiation activity, or a nucleic acid sequence of any one of SEQ ID NOs: 1-27; and (b) a first polynucleotide of interest. In some embodiments, the polynucleotide encodes a morphogen, a transforming protein, a recombination modulator, a repair modulator, a defense pathway modulator, a nuclease, a selectable marker, and/or a regulatory RNA. In some embodiments, said first polynucleotide of interest comprises a nucleic acid sequence that encodes: (i) a polypeptide comprising an amino acid sequence having at least 80% sequence identity to SEQ ID NO: 44 or SEQ ID NO: 45, wherein said polynucleotide has morphogenic activity; or (ii) a polypeptide comprising an amino acid sequence of SEQ ID NO: 44 or SEQ ID NO: 45. In some embodiments, the regulatory RNA is one or more of a microRNA (miRNA), a short-hairpin RNA, a guide RNA, a transposase, a homology-directed repair enhancer, and a non-homologous end-joining suppressor. In some embodiments, the promoter molecule further comprises a 5′UTR sequence, a 5′UTR intron sequence, an exon sequence from a coding region, and/or an intron sequence from a coding region of a plant genome.

In some embodiments, the DNA construct of the present disclosure further comprises a second promoter molecule operably linked to a second polynucleotide of interest. In some embodiments, the first and second polynucleotides of interest each encode a morphogen.

In some embodiments, the first polynucleotide of interest operably linked to the first promoter molecule comprises a nucleic acid sequence that encodes: (i) a polypeptide comprising an amino acid sequence that shares at least 80% sequence identity with SEQ ID NO: 44, wherein said polypeptide has morphogenic activity, or (ii) a polypeptide comprising an amino acid sequence of SEQ ID NO: 44; and wherein the second polynucleotide of interest operably linked to the second promoter molecule comprises a nucleic acid sequence that encodes: (iii) a polypeptide comprising an amino acid sequence that shares at least 80% sequence identity with SEQ ID NO: 45, wherein said polypeptide has morphogenic activity, or (iv) a polypeptide comprising an amino acid sequence of SEQ ID NO: 45.

In some embodiments, the first polynucleotide of interest operably linked to the first promoter molecule comprises a nucleic acid sequence that encodes: (i) a polypeptide comprising an amino acid sequence that shares at least 80% sequence identity with SEQ ID NO: 45, wherein said polypeptide has morphogenic activity, or (ii) a polypeptide comprising an amino acid sequence of SEQ ID NO: 45; and wherein the second polynucleotide of interest operably linked to the second promoter molecule comprises a nucleic acid sequence that encodes: (iii) a polypeptide comprising an amino acid sequence that shares at least 80% sequence identity with SEQ ID NO: 44, wherein said polypeptide has morphogenic activity, or (iv) a polypeptide comprising an amino acid sequence of SEQ ID NO: 44.

In some embodiments, the first promoter molecule comprises a nucleic acid sequence that shares at least 80% sequence identity with any one of SEQ ID NOs: 1-3 and retains transcription initiation activity, or a nucleic acid sequence of any one of SEQ ID NOs: 1-3.

In some embodiments, the second promoter molecule comprises a nucleic acid sequence that shares at least 80% sequence identity with any one of SEQ ID NOs: 1-27 and retains transcription initiation activity, or a nucleic acid sequence of any one of SEQ ID NOs: 1-27. In some embodiments, said promoter molecule(s) comprising a nucleic acid sequence that shares at least 80% sequence identity with any one of SEQ ID NOs: 1-27 and retains transcription initiation activity, or a nucleic acid sequence of any one of SEQ ID NOs: 1-27, initiates expression of the first polynucleotide of interest limited to a seed-to-seedling developmental phase when the DNA construct is introduced in a plant or plant part. In some embodiments, said promoter molecule(s) comprising a nucleic acid sequence that shares at least 80% sequence identity with any one of SEQ ID NOs: 1-27 and retains transcription initiation activity, or a nucleic acid sequence of any one of SEQ ID NOs: 1-27, initiates embryonic tissue-preferred expression of the first polynucleotide of interest when the DNA construct is introduced in a plant or plant part. In some embodiments, the preferred embryonic tissue is epicotyl, hypocotyl, radicle, cotyledon, or a combination thereof.

In some embodiments, the present disclosure provides cells comprising the DNA construct of the present disclosure. In some embodiments, the cell is a plant cell.

In some embodiments, the present disclosure provides plants or plant parts comprising the DNA construct comprising the promoter molecule of the present disclosure or the cell of the present disclosure.

In some embodiments, said plant of the present disclosure is selected from the group consisting of corn (Zea mays), Brassica species, Brassica napus, Brassica rapa, Brassica juncea, alfalfa (Medicago sativa), pea (Pisum sativum), fava bean (Vicia faba), common bean (Phaseolus vulgaris), chickpea (Cicer arietinum), mung bean (Vigna radiata), white lupin (Lupinus albus), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. In some embodiments, said plant is a dicot. In some embodiments, said plant is a legume.

In some aspects, provided herein is a method of expressing a polynucleotide of interest in a plant or plant part comprising introducing a DNA construct into said plant or plant part, wherein the DNA construct comprises, in operable linkage: (a) a first promoter molecule comprising a nucleic acid sequence that shares at least 80% sequence identity with any one of SEQ ID NOs: 1-27 and retains transcription initiation activity, or a nucleic acid sequence of any one of SEQ ID NOs: 1-27; and (b) a first polynucleotide of interest.

In some aspects, provided herein is a method of transforming a plant or plant part, comprising: introducing a DNA construct into a plant cell, wherein the DNA construct comprises, in operable linkage: (a) a first promoter molecule comprising a nucleic acid sequence that shares at least 80% sequence identity with any one of SEQ ID NOs: 1-27 and retains transcription initiation activity, or a nucleic acid sequence of any one of SEQ ID NOs: 1-27; and (b) a first polynucleotide of interest; and regenerating a transformed plant or plant part from said plant cell. In some embodiments, the method increases normal shoot formation, frequency of shoot producing plants or plant parts, and/or number of regenerated shoots from transformed plants or plant parts relative to a control method comprising introducing a control DNA construct comprising a control promoter molecule into a plant cell. In some embodiments, the frequency of shoot producing plants or plant parts is increased by about 10% to about 500% relative to a control method. In some embodiments, the number of regenerated shoots from transformed plants or plant parts is increased by about 10% to about 1200% relative to a control method.

In some embodiments of the methods of present disclosure, the first polynucleotide of interest encodes a morphogen, a transforming protein, a recombination modulator, a repair modulator, a defense pathway modulator, a nuclease, a selectable marker, and/or a regulatory RNA. In some embodiments, said first polynucleotide of interest comprises a nucleic acid sequence that encodes: (i) a polypeptide comprising an amino acid sequence having at least 80% sequence identity to SEQ ID NO: 44 or SEQ ID NO: 45, wherein said polypeptide has morphogenic activity; or (ii) a polypeptide comprising an amino acid sequence of SEQ ID NO: 44 or SEQ ID NO: 45. In some embodiments, the regulatory RNA is one or more of a microRNA (miRNA), a short-hairpin RNA, a guide RNA, a transposase, a homology-directed repair enhancer, and a non-homologous end-joining suppressor. In some embodiments, the first promoter molecule further comprises a 5′UTR sequence, a 5′UTR intron sequence, an exon sequence from a coding region, and/or an intron sequence from a coding region of a plant genome.

In some embodiments, the DNA construct further comprises a second promoter molecule operably linked to a second polynucleotide of interest. In some embodiments, the methods of the present disclosure further comprise introducing a second DNA construct into said plant or plant part, wherein the second DNA construct comprises a second promoter molecule operably linked to a second polynucleotide of interest. In some embodiments, the first and second polynucleotides of interest each encode a morphogen.

In some embodiments, the first polynucleotide of interest operably linked to the first promoter molecule comprises a nucleic acid sequence that encodes: (i) a polypeptide comprising an amino acid sequence that shares at least 80% sequence identity with SEQ ID NO: 44, wherein said polypeptide has morphogenic activity, or (ii) a polypeptide comprising an amino acid sequence of SEQ ID NO: 44; and wherein the second polynucleotide of interest operably linked to the second promoter molecule comprises a nucleic acid sequence that encodes: (iii) a polypeptide comprising an amino acid sequence that shares at least 80% sequence identity with SEQ ID NO: 45, wherein said polypeptide has morphogenic activity, or (iv) a polypeptide comprising an amino acid sequence of SEQ ID NO: 45.

In some embodiments, the first polynucleotide of interest of the first DNA construct comprises a nucleic acid sequence that encodes: (i) a polypeptide comprising an amino acid sequence having at least 80% sequence identity to SEQ ID NO: 45, wherein said polypeptide has morphogenic activity; or (ii) a polypeptide comprising an amino acid sequence of SEQ ID NO: 45; and wherein the second polynucleotide of interest of the second DNA construct comprises a nucleic acid sequence that encodes: (iii) a polypeptide comprising an amino acid sequence having at least 80% sequence identity to SEQ ID NO: 44, wherein said polypeptide has morphogenic activity; or (iv) a polypeptide comprising an amino acid sequence of SEQ ID NO: 44.

In some embodiments, the first promoter molecule comprises a nucleic acid sequence that shares at least 80% sequence identity with any one of SEQ ID NOs: 1-3 and retains transcription initiation activity, or a nucleic acid sequence of any one of SEQ ID NOs: 1-3.

In some embodiments, the second promoter molecule comprises a nucleic acid sequence that shares at least 80% sequence identity with any one of SEQ ID NOs: 1-27 and retains transcription initiation activity, or a nucleic acid sequence of any one of SEQ ID NOs: 1-27. In some embodiments, said promoter molecule(s) comprising a nucleic acid sequence that shares at least 80% sequence identity with any one of SEQ ID NOs: 1-27 and retains transcription initiation activity, or a nucleic acid sequence of any one of SEQ ID NOs: 1-27, initiates expression of the polynucleotide of interest limited to a seed-to-seedling developmental phase in the plant or plant part. In some embodiments, the said promoter molecule(s) comprising a nucleic acid sequence that shares at least 80% sequence identity with any one of SEQ ID NOs: 1-27 and retains transcription initiation activity, or a nucleic acid sequence of any one of SEQ ID NOs: 1-27, initiates embryonic tissue-preferred expression of the polynucleotide of interest in the plant or plant part. In some embodiments, the preferred embryonic tissue is epicotyl, hypocotyl, radicle, cotyledon, or a combination thereof.

In some embodiments of the methods of present disclosure, said plant is selected from the group consisting of corn (Zea mays), Brassica species, Brassica napus, Brassica rapa, Brassica juncea, alfalfa (Medicago sativa), pea (Pisum sativum), fava bean (Vicia faba), common bean (Phaseolus vulgaris), chickpea (Cicer arietinum), mung bean (Vigna radiata), white lupin (Lupinus albus), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. In some embodiments, said plant is a dicot. In some embodiments, said plant is a legume.

In some embodiments of the methods of the present disclosure, the polynucleotide of interest is stably inserted into a genome of said plant or plant part.

The present disclosure provides plants or plant parts produced by the method of the present disclosure, wherein said plant or plant part comprises said DNA construct.

### BRIEF DESCRIPTION OF THE DRAWINGS

FIG. 1 depicts sequence diagrams of spatio-temporal promoters, ST1p, ST2p, and ST3p as items A, B, and C, respectively. The nucleic acid sequences of ST1p, ST2p, and ST3p are set forth as SEQ ID NOs: 1, 2, and 3, respectively.

FIG. 2 schematically depicts responses of explants after introduction of a polynucleotide of interest. FIG. 2A represents formation of a normal shoot (“shoot”). FIG. 2B represents formation of multiple immature shoots that are not transplantable (“morphogenic”). FIG. 2C represents formation of multiple normal shoots (“multiple shoots”).

FIG. 3 depicts exemplary shooting responses of pea explants stably transformed (using Agrobacterium tumefaciens). FIG. 3A depicts a transformant without morphogens. FIG. 3B depicts a transformant with constitutively-expressed morphogens. FIG. 3C depicts a transformant with morphogens, one of which is operably linked to a spatio-temporal promoter.

FIG. 4 depicts percentages of explants producing shoots in TO pea plants stably transformed via Agrobacterium with morphogens operably linked to a spatio-temporal promoter or a constitutive promoter, or the negative control, as described in the figure.

FIG. 5 depicts percentages of explants producing shoots in TO pea plants stably transformed via Agrobacterium with morphogens operably linked to a spatio-temporal promoter or a constitutive promoter, or the negative control, as described in the figure.

FIG. 6 depicts exemplary shooting responses of soybean explants stably transformed (using Agrobacterium tumefaciens). FIG. 6A depicts a transformant without morphogens. FIG. 6B depicts a transformant with constitutively-expressed morphogens. FIG. 6C depicts a transformant with morphogens, one of which is operably linked to a spatio-temporal promoter.

FIG. 7 depicts percentages of explants producing shoots in TO soybean plants stably transformed via Agrobacterium with morphogens operably linked to a spatio-temporal promoter or a constitutive promoter, or the negative control, as described in the figure.

FIG. 8 depicts percentages of explants producing shoots in TO soybean plants stably transformed via Agrobacterium with morphogens operably linked to a spatio-temporal promoter or a constitutive promoter, or the negative control, as described in the figure.

FIG. 9 depicts percentages of explants producing shoots in TO soybean plants with morphogens operably linked to a spatio-temporal promoter or a constitutive promoter, or the negative control, transformed stably (or transiently) via Agrobacterium as described in the figure.

### DETAILED DESCRIPTION OF THE INVENTION

The present disclosure now will be described more fully hereinafter. The disclosure may be embodied in many different forms and should not be construed as limited to the aspects set forth herein; rather, these aspects are provided so that this disclosure will satisfy applicable legal requirements.

### I. Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an,” or “the” can mean one or more than one. For example, “a” cell can mean a single cell or a multiplicity of cells. Further, the term “a plant” may include a plurality of plants.

As used herein, unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. The recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

A “plant” refers to a whole plant, any part thereof, or a cell or tissue culture derived from a plant, comprising any of: whole plants, plant components or organs (e.g., leaves, stems, roots, embryos, pollen, ovules, seeds, grains, leaves, flowers, branches, fruit, pulp, juice, kernels, ears, cobs, husks, stalks, root tips, anthers, etc.), plant tissues, plant cell tissue cultures from which plants can be regenerated, plant calli, plant clumps, seeds, plant cells, protoplasts and/or progeny of the same. A plant cell is a biological cell of a plant, taken from a plant or derived through culture of a cell taken from a plant. Grain is intended to mean the mature seed produced by commercial growers for purposes other than growing or reproducing the species. Progeny, variants, and mutants of the regenerated plants comprising the introduced polynucleotides are also within the scope of the invention. Further provided is a processed plant product (e.g., extract) or byproduct that retains one or more polynucleotides disclosed herein.

As used herein, a “subject plant or plant cell” is one in which genetic alteration, such as a mutation, has been effected as to a polynucleotide of interest, or is a plant or plant cell which is descended from a plant or cell so altered and which comprises the alteration. As used herein, the term “mutated” or “genetically modified” or “transgenic” or “transformed” or “edited” plants, plant cells, plant tissues, plant parts or seeds refers plants, plant cells, plant tissues, plant parts or seeds that have been mutated by the methods of the present disclosure to include one or more mutations (e.g., insertions, substitutions, or deletions) in the genomic sequence.

As used herein, a “control plant” or “control plant part” or “control cell” or “control seed” refers to a plant or plant part or plant cell or seed that has not been subject to the methods and compositions described herein. A “control” or “control plant” or “control plant part” or “control cell” or “control seed” provides a reference point for measuring changes in phenotype of the subject plant or plant cell. A control plant or plant cell may comprise, for example: (a) a control promoter with reference to the promoters of the present disclosure; (b) a wild-type plant or cell, i.e., of the same genotype as the starting material for the genetic alteration which resulted in the subject plant or cell; (c) a plant or plant cell of the same genotype as the starting material but which has been transformed with a null construct (i.e. with a construct which has no known effect on the trait of interest, such as a construct comprising a marker gene); (d) a plant or plant cell which is a non-transformed segregant among progeny of a subject plant or plant cell; (e) a plant or plant cell genetically identical to the subject plant or plant cell but which is not exposed to conditions or stimuli (e.g., sucrose) that would induce expression of the polynucleotide of interest; or (f) the subject plant or plant cell itself, under conditions in which the polynucleotide of interest is not expressed. In certain instances, a control plant of the present disclosure is grown under the same environmental conditions (e.g., same or similar temperature, humidity, air quality, soil quality, water quality, and/or pH conditions) as a subject plant described herein. Similarly, a control protein or control protein composition can refer to a protein or protein composition that is isolated or derived from a control plant. In specific embodiments, a control plant, plant part, or plant cell is a plant, plant part, or plant cell that comprises a control promoter molecule or does not comprise the promoter molecule of the present disclosure.

Plant cells possess nuclear, plastid, and mitochondrial genomes. The compositions and methods of the present invention may be used to modify the sequence of the nuclear, plastid, and/or mitochondrial genome, or may be used to modulate the expression of a gene or genes encoded by the nuclear, plastid, and/or mitochondrial genome. Accordingly, by “chromosome” or “chromosomal” is intended the nuclear, plastid, or mitochondrial genomic DNA. “Genome” as it applies to plant cells encompasses not only chromosomal DNA found within the nucleus, but organelle DNA found within subcellular components (e.g., mitochondria or plastids) of the cell.

As used herein, the term “gene” or “coding sequence”, herein used interchangeably, refers to a functional nucleic acid unit encoding a protein, polypeptide, or peptide. As will be understood by those in the art, this functional term includes genomic sequences, cDNA sequences, and smaller engineered gene segments that express, or may be adapted to express proteins, polypeptides, domains, peptides, fusion proteins, and mutants.

As used herein, the term a “nucleic acid”, used interchangeably with a “nucleotide”, refers to a molecule consisting of a nucleoside and a phosphate that serves as a component of DNA or RNA. For instance, nucleic acids include adenine, guanine, cytosine, uracil, and thymine.

As used herein, a “mutation” is any change in a nucleic acid sequence. Nonlimiting examples comprise insertions, deletions, duplications, substitutions, inversions, and translocations of any nucleic acid sequence, regardless of how the mutation is brought about and regardless of how or whether the mutation alters the functions or interactions of the nucleic acid. For example, and without limitation, a mutation may produce altered enzymatic activity of a ribozyme, altered base pairing between nucleic acids (e.g. RNA interference interactions, DNA-RNA binding, etc.), altered mRNA folding stability, and/or how a nucleic acid interacts with polypeptides (e.g. DNA-transcription factor interactions, RNA-ribosome interactions, guide RNA-endonuclease reactions, etc.). A mutation might result in the production of proteins with altered amino acid sequences (e.g. missense mutations, nonsense mutations, frameshift mutations, etc.) and/or the production of proteins with the same amino acid sequence (e.g. silent mutations). Certain synonymous mutations may create no observed change in the plant while others that encode for an identical protein sequence nevertheless result in an altered plant phenotype (e.g. due to codon usage bias, altered secondary protein structures, etc.). Mutations may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements such as tRNAs, ribozymes, ribosome components, and microRNAs.

Accordingly, “plant with a mutation” or “plant part with a mutation” or “plant cell with a mutation” or “plant genome with a mutation” refers to a plant or plant part or plant cell or plant genome that contains a mutation (e.g., an insertion, a substitution, or a deletion) described in the present disclosure.

“Genome editing” or “gene editing” as used herein refers to a type of genetic engineering by which one or more mutations (e.g., insertions, substitutions, deletions, modifications) are introduced at a specific location of the genome. “Editing reagents”, as used herein, refers to a set of molecules or a construct comprising or encoding the molecules for introducing one or more mutations in the genome. Exemplary editing reagents comprise a nuclease and a guide RNA. For example, a CRISPR (clustered regularly interspaced short palindromic repeats) system comprises a CRISPR nuclease [e.g., CRISPR-associated (Cas) endonuclease or a variant thereof, such as Cas12a] and a guide RNA. A CRISPR nuclease associates with a guide RNA that directs nucleic acid cleavage by the associated endonuclease by hybridizing to a recognition site in a polynucleotide. The guide RNA comprises a direct repeat and a guide sequence, which is complementary to the target recognition site. In certain embodiments, the CRISPR system further comprises a tracrRNA (trans-activating CRISPR RNA) that is complementary (fully or partially) to the direct repeat sequence present on the guide RNA. A “TALEN” nuclease is an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, and yeast HO endonuclease. A “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, and yeast HO endonuclease.

As used herein, the terms “nuclease” and “endonuclease” are used interchangeably to refer to naturally-occurring or engineered enzymes, which cleave a phosphodiester bond within a polynucleotide chain.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory molecules and polynucleotides that are not found together in nature. For example, a recombinant DNA construct may comprise regulatory molecules and polynucleotides that are derived from different sources, or regulatory molecules and polynucleotides derived from the same source and arranged in a manner different than that found in nature. Such a construct may be used by itself or may be used in conjunction with a vector. In specific embodiments, a recombinant DNA construct or expression cassette comprises a promoter operably linked to a polynucleotide of interest, wherein the promoter is heterologous to the polynucleotide of interest.

An expression construct can permit transcription of a particular polynucleic acid sequence in a host cell (e.g., a bacterial cell or a plant cell). An expression cassette may be part of a plasmid, viral genome, or nucleic acid fragment. Typically, an expression cassette includes a polynucleotide to be transcribed, operably linked to a promoter. “Operably linked” is intended to mean a functional linkage between two or more elements. For example, an operable linkage between a promoter of the present invention and a heterologous nucleotide is a functional link that allows for expression of the heterologous nucleic acid molecule. Operably linked elements may be contiguous or non-contiguous. When used to refer to the joining of two protein coding regions, by operably linked is intended that the coding regions are in the same reading frame. The cassette may additionally contain at least one additional polynucleotide for co-transforming into the plant. Alternatively, the additional polynucleotide(s) can be provided on multiple expression cassettes or DNA constructs. Such an expression cassette or construct is provided with a plurality of restriction sites and/or recombination sites for insertion of the heterologous nucleotide sequence of interest to be under the transcriptional regulation of the promoter regions of the invention. The expression cassette may additionally contain selectable marker genes. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

As used herein, “function” of a gene, a polynucleotide, a peptide, a protein, or a molecule refers to activity of a gene, a polynucleotide, a peptide, a protein, or a molecule. For example, the function of a morphogen may be assessed by developmental phenotypes of plants or plant parts comprising the morphogen, e.g., number and form of shoot formation in the plants or plant parts.

As used herein, the term “expression” or “expressing” refers to the transcription and/or translation of a particular nucleic acid sequence driven by a promoter.

“Introduced” in the context of inserting a nucleic acid molecule (e.g., a DNA construct comprising a promoter molecule and a polynucleotide sequence of interest) into a cell, a plant, or a plant part means “transfection” or “transformation” or “transduction” and includes reference to the incorporation of a nucleic acid fragment into a plant cell where the nucleic acid fragment may be incorporated into the genome of the cell (e.g., nuclear chromosome, plasmid, plastid chromosome or mitochondrial chromosome), converted into an autonomous replicon, or transiently expressed (e.g., transfected mRNA).

As used herein with respect to a parameter, the term “decreased” or “decreasing” or “decrease” or “reduced” or “reducing” or “reduce” or “lower” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter from a comparison control, e.g., an established normal or reference level of the parameter, or an established standard control. Accordingly, the terms “decreased”, “reduced”, and the like encompass both a partial reduction and a complete reduction compared to a control.

As used herein with respect to a parameter, the term “increased” or “increasing” or “increase” or “enhanced” or “enhancing” or “enhance” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or 1000%) positive change in the parameter from a comparison control, e.g., an established normal or reference level of the parameter, or an established standard control.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the term “polypeptide” refers to a linear organic polymer containing a large number of amino-acid residues bonded together by peptide bonds in a chain, forming part of (or the whole of) a protein molecule. The amino acid sequence of the polypeptide refers to the linear consecutive arrangement of the amino acids comprising the polypeptide, or a portion thereof.

As used herein the term “polynucleotide” refers to a single or double stranded nucleic acid sequence which is isolated and provided in the form of an RNA sequence (e.g., an mRNA sequence), a complementary polynucleic acid sequence (cDNA), a genomic polynucleic acid sequence and/or a composite polynucleic acid sequences (e.g., a combination of the above).

The term “isolated” refers to at least partially separated from the natural environment e.g., from a plant cell.

As used herein, the terms “exogenous” or “heterologous” in reference to a nucleic acid sequence or amino acid sequence are intended to mean a sequence that is purely synthetic, that originates from a foreign species, or, if from the same species, is substantially modified from its native form in composition and/or genomic locus by deliberate human intervention. Thus, a heterologous nucleic acid sequence may not be naturally expressed within the plant (e.g., a nucleic acid sequence from a different species) or may have altered expression when compared to the corresponding wild type plant. An exogenous polynucleotide may be introduced into the plant in a stable or transient manner, so as to produce a ribonucleic acid (RNA) molecule and/or a polypeptide molecule. It should be noted that the exogenous polynucleotide may comprise a nucleic acid sequence which is identical or partially homologous to an endogenous nucleic acid sequence of the plant.

As used herein, the term “endogenous” in reference to a gene or nucleic acid or protein is intended to mean a gene or nucleic acid or protein that is naturally comprised within or expressed by a cell. Endogenous genes can include genes that naturally occur in the cell of a plant, but that have been modified in the genome of the cell without insertion or replacement of a heterologous gene that is from another plant species or another location within the genome of the modified cell.

As used herein, “fertilization” and/or “crossing” broadly includes bringing the genomes of gametes together to form zygotes but also broadly may include pollination, syngamy, fecundation and other processes related to sexual reproduction. Typically, a cross and/or fertilization occurs after pollen is transferred from one flower to another, but those of ordinary skill in the art will understand that plant breeders can leverage their understanding of fertilization and the overlapping steps of crossing, pollination, syngamy, and fecundation to circumvent certain steps of the plant life cycle and yet achieve equivalent outcomes, for example, a plant or cell of a soybean cultivar described herein. In certain embodiments, a user of this innovation can generate a plant of the claimed invention by removing a genome from its host gamete cell before syngamy and inserting it into the nucleus of another cell. While this variation avoids the unnecessary steps of pollination and syngamy and produces a cell that may not satisfy certain definitions of a zygote, the process falls within the definition of fertilization and/or crossing as used herein when performed in conjunction with these teachings. In certain embodiments, the gametes are not different cell types (i.e. egg vs. sperm), but rather the same type and techniques are used to effect the combination of their genomes into a regenerable cell. Other embodiments of fertilization and/or crossing include circumstances where the gametes originate from the same parent plant, i.e. a “self” or “self-fertilization”. While selfing a plant does not require the transfer pollen from one plant to another, those of skill in the art will recognize that it nevertheless serves as an example of a cross, just as it serves as a type of fertilization. Thus, methods and compositions taught herein are not limited to certain techniques or steps that must be performed to create a plant or an offspring plant of the claimed invention, but rather include broadly any method that is substantially the same and/or results in compositions of the claimed invention.

“Homolog” or “homologous sequence” may refer to both orthologous and paralogous sequences. Paralogous sequence relates to gene-duplications within the genome of a species. Orthologous sequence relates to homologous genes in different organisms due to ancestral relationship. Thus, orthologs are evolutionary counterparts derived from a single ancestral gene in the last common ancestor of given two species and therefore have great likelihood of having the same function. One option to identify homologs (e.g., orthologs) in monocot plant species is by performing a reciprocal BLAST search. This may be done by a first blast involving blasting the sequence-of-interest against any sequence database, such as the publicly available NCBI database which may be found at: ncbi.nlm.nih.gov. If orthologs in rice were sought, the sequence-of-interest would be blasted against, for example, the 28,469 full-length cDNA clones from Oryza sativa Nipponbare available at NCBI. The blast results may be filtered. The full-length sequences of either the filtered results or the non-filtered results are then blasted back (second blast) against the sequences of the organism from which the sequence-of-interest is derived. The results of the first and second blasts are then compared. An ortholog is identified when the sequence resulting in the highest score (best hit) in the first blast identifies in the second blast the query sequence (the original sequence-of-interest) as the best hit. Using the same rational a paralog (homolog to a gene in the same organism) is found. In case of large sequence families, the ClustalW program may be used [ebi.ac.uk/Tools/clustalw2/index.html], followed by a neighbor-joining tree (wikipedia.org/wiki/Neighbor-joining) which helps visualizing the clustering.

In some embodiments, the term “homolog” as used herein, refers to functional homologs of genes. A functional homolog is a gene encoding a polypeptide that has sequence similarity to a polypeptide encoded by a reference gene, and the polypeptide encoded by the homolog carries out one or more of the biochemical or physiological function(s) of the polypeptide encoded by the reference gene. In general, it is preferred that functional homologs and/or polypeptides encoded by functional homologs share at least some degree of sequence identity with the reference gene or polypeptide encoded by the reference gene. Homology (e.g., percent homology, sequence identity+sequence similarity) can be determined using any homology comparison software computing a pairwise sequence alignment.

As used herein, “sequence identity,” “identity,” “percent identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. The determination of percent sequence identity between any two sequences can be accomplished using a mathematical algorithm, or a computer implementation thereof. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms for comparison of sequences to determine sequence identity include, but are not limited to: CLUSTAL in the PC/Gene program (available from Intelligenetics, Mountain View, California); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, California, USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection. Identity (e.g., percent homology) can be determined using any homology comparison software, including for example, the BlastN software of the National Center of Biotechnology Information (NCBI) such as by using default parameters.

According to some embodiments, the identity is a global identity, i.e., an identity over the entire amino acid or nucleic acid sequences of the invention and not over portions thereof.

According to some embodiments, the term “homology” or “homologous” refers to identity of two or more nucleic acid sequences; or identity of two or more amino acid sequences; or the identity of an amino acid sequence to one or more nucleic acid sequence. According to some embodiments, the homology is a global homology, e.g., a homology over the entire amino acid or nucleic acid sequences of the invention and not over portions thereof. The degree of homology or identity between two or more sequences can be determined using various known sequence comparison tools which are described in WO2014/102774.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “population” refers to a set comprising any number, including one, of individuals, objects, or data from which samples are taken for evaluation, e.g., estimating quantitative trait locus (QTL) associations and/or disease tolerance. Most commonly, the terms relate to a breeding population of plants from which members are selected and crossed to produce progeny in a breeding program. A population of plants can include the progeny of a single breeding cross or a plurality of breeding crosses and can be either actual plants or plant derived material, or in silico representations of plants. The member of a population need not be identical to the population members selected for use in subsequent cycles of analyses, nor does it need to be identical to those population members ultimately selected to obtain a final progeny of plants. Often, a plant population is derived from a single biparental cross but can also derive from two or more crosses between the same or different parents. Although a population of plants can comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population in a plant breeding program.

As used herein, the term “crop performance” is used synonymously with “plant performance” and refers to of how well a plant grows under a set of environmental conditions and cultivation practices. Crop performance can be measured by any metric a user associates with a crop's productivity (e.g., yield), appearance and/or robustness (e.g., color, morphology, height, biomass, maturation rate, etc.), product quality (e.g., fiber lint percent, fiber quality, seed protein content, seed carbohydrate content, etc.), cost of goods sold (e.g., the cost of creating a seed, plant, or plant product in a commercial, research, or industrial setting) and/or a plant's tolerance to disease (e.g., a response associated with deliberate or spontaneous infection by a pathogen) and/or environmental stress (e.g., drought, flooding, low nitrogen or other soil nutrients, wind, hail, temperature, day length, etc.). Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g., the volume or weight of seed or other plant product measured in liters or grams) or some other metric assigned to some aspect of a plant that can be represented on a scale (e.g., assigning a 1-10 value to a plant based on its disease tolerance).

A “microbe” will be understood to be a microorganism, i.e. a microscopic organism, which can be single celled or multicellular. Microorganisms are very diverse and include all the bacteria, archaea, protozoa, fungi, and algae, especially cells of plant pathogens and/or plant symbionts. Certain animals are also considered microbes, e.g. rotifers. In various embodiments, a microbe can be any of several different microscopic stages of a plant or animal. Microbes also include viruses, viroids, and prions, especially those which are pathogens or symbionts to crop plants. A “pathogen” as used herein refers to a microbe that causes disease or harmful effects on plant health.

A “fungus” includes any cell or tissue derived from a fungus, for example whole fungus, fungus components, organs, spores, hyphae, mycelium, and/or progeny of the same. A fungus cell is a biological cell of a fungus, taken from a fungus or derived through culture of a cell taken from a fungus.

A “pest” is any organism that can affect the performance of a plant in an undesirable way. Common pests include microbes, animals (e.g. insects and other herbivores), and/or plants (e.g. weeds). Thus, a pesticide is any substance that reduces the survivability and/or reproduction of a pest, e.g. fungicides, bactericides, insecticides, herbicides, and other toxins.

“Tolerance” or “improved tolerance” in a plant to disease conditions (e.g. growing in the presence of a pest) will be understood to mean an indication that the plant is less affected by the presence of pests and/or disease conditions with respect to yield, survivability and/or other relevant agronomic measures, compared to a less tolerant, more “susceptible” plant. Tolerance is a relative term, indicating that a “tolerant” plant survives and/or performs better in the presence of pests and/or disease conditions compared to other (less tolerant) plants (e.g., a different soybean cultivar) grown in similar circumstances. As used in the art, “tolerance” is sometimes used interchangeably with “resistance”, although resistance is sometimes used to indicate that a plant appears maximally tolerant to, or unaffected by, the presence of disease conditions. Plant breeders of ordinary skill in the art will appreciate that plant tolerance levels vary widely, often representing a spectrum of more-tolerant or less-tolerant phenotypes, and are thus trained to determine the relative tolerance of different plants, plant lines or plant families and recognize the phenotypic gradations of tolerance.

“Yield” as used herein is defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance, photosynthetic carbon assimilation rates, and early vigor may also be important factors in determining yield. Optimizing the abovementioned factors may therefore contribute to increasing crop yield. Yield can be measured and expressed by any means known in the art. In specific embodiments, yield is measured by seed weight or volume in a given harvest area.

A plant, or its environment, can be contacted with a wide variety of “agriculture treatment agents.” As used herein, an “agriculture treatment agent”, or “treatment agent”, or “agent” can refer to any exogenously provided compound that can be brought into contact with a plant tissue (e.g. a seed) or its environment that affects a plant's growth, development and/or performance, including agents that affect other organisms in the plant's environment when those effects subsequently alter a plant's performance, growth, and/or development (e.g. an insecticide that kills plant pathogens in the plant's environment, thereby improving the ability of the plant to tolerate the insect's presence). Agriculture treatment agents also include a broad range of chemicals and/or biological substances that are applied to seeds, in which case they are commonly referred to as seed treatments and/or seed dressings. Seed treatments are commonly applied as either a dry formulation or a wet slurry or liquid formulation prior to planting and, as used herein, generally include any agriculture treatment agent including growth regulators, micronutrients, nitrogen-fixing microbes, and/or inoculants. Agriculture treatment agents include pesticides (e.g. fungicides, insecticides, bactericides, etc.) hormones (abscisic acids, auxins, cytokinins, gibberellins, etc.) herbicides (e.g. glyphosate, atrazine, 2,4-D, dicamba, etc.), nutrients (e.g. a plant fertilizer), and/or a broad range of biological agents, for example a seed treatment inoculant comprising a microbe that improves crop performance, e.g. by promoting germination and/or root development. In certain embodiments, the agriculture treatment agent acts extracellularly within the plant tissue, such as interacting with receptors on the outer cell surface. In some embodiments, the agriculture treatment agent enters cells within the plant tissue. In certain embodiments, the agriculture treatment agent remains on the surface of the plant and/or the soil near the plant. In certain embodiments, the agriculture treatment agent is contained within a liquid. Such liquids include, but are not limited to, solutions, suspensions, emulsions, and colloidal dispersions. In some embodiments, liquids described herein will be of an aqueous nature. However, in various embodiments, such aqueous liquids that comprise water can also comprise water insoluble components, can comprise an insoluble component that is made soluble in water by addition of a surfactant, or can comprise any combination of soluble components and surfactants. In certain embodiments, the application of the agriculture treatment agent is controlled by encapsulating the agent within a coating, or capsule (e.g. microencapsulation). In certain embodiments, the agriculture treatment agent comprises a nanoparticle and/or the application of the agriculture treatment agent comprises the use of nanotechnology. In some embodiments, the plants described herein can grow in the presence of one or more agricultural treatment agents. For example, the plants described herein can have an increased expression of the polynucleotide of interest, e.g., a guide RNA or a nuclease, or mutations in the genome introduced by such editing reagents, and can grow in the presence of commonly used herbicides.

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

All publications, patent applications, patents, and other references mentioned herein are incorporated by reference herein in their entirety.

### II. Overview of the Invention

Certain polynucleotides of interest are desired for expression limited to specific growth stages and/or tissue types, particularly in cases where their constitutive expression has negative consequences on plant physiology. Such polynucleotides of interest may include those encoding morphogens, transposes, other molecules that may be desired for regulated expression in plants, but their constitutive expression potentially has fitness-compromising impacts, and molecules whose toxicity has prevented further research, but may be useful when expressed in a spatially, temporally, and/or spatio-temporally specific manner.

Accordingly, disclosed herein are promoter molecules capable of driving expression of operably-linked polynucleotides of interest (e.g., transgenes) in a spatially, temporally, and/or spatio-temporally specific manner, i.e., in specific tissue and/or in a specific timeframe/phase of plants' life cycle. Associated compositions and methods for expressing a polynucleotide of interest in a plant or plant part using a spatio-temporal promoter provided herein are also provided. In some aspects, the promoters of the present disclosure are active in the specific tissue and/or the specific timeframe, and/or are inactive outside the specific tissue and/or outside the specific timeframe. The advantage of such self-regulatory spatio-temporal promoter approach of the present disclosure, relative to the common alternative of inducible expression using inducible promoters, includes the automatic activation of the expression of operably linked polynucleotides by the plant, thereby avoiding the skilled labor needed to exogenously regulate activity of the polynucleotides of interest.

The promoter molecules of the present disclosure can comprise the nucleic acid sequence for a soybean XCP promoter, soybean DUF1118 promoter, soybean T5AH promoter, pea XCP promoter, medicago XCP promoter, pea DUF1118 promoter, medicago DUF1118 promoter, pea T5AH promoter, medicago T5AH promoter, tomato XCP-LIKE promoter, Arachis hypogaea XCP-1 promoter, Arachis hypogaea XCP-2 promoter, Cicer arietinum XCP-1 promoter, Cicer arietinum XCP-2 promoter, Lupinus albus XCP-1 promoter, Lotus japonicus XCP-1 promoter, Phaseolus acutifolius XCP-1 promoter, Phaseolus acutifolius XCP-2 promoter, Phaseolus lunatus XCP-1 promoter, Phaseolus vulgaris XCP-1 promoter, Phaseolus vulgaris XCP-2 promoter, Trifolium pratense XCP-1 promoter, Trifolium pratense XCP-2 promoter, Vigna unguiculata XCP-1 promoter, Vigna unguiculata XCP-2 promoter, optionally including a 5′UTR sequence, a 5′UTR intron sequence, an exon sequence from a coding region, and/or an intron sequence from a coding region. The promoter molecules can also comprise a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-27, or a nucleic acid sequence of any one of SEQ ID NOs: 1-27. The promoters of the present disclosure can be used to express any polynucleotides of interest in plants or plant parts, including those encoding molecules that facilitate transformation and/or gene editing, but whose prolonged expression can prevent healthy, fertile plants to be regenerated or produced. The promoters of the present disclosure can be used to express a polynucleotide of interest limited to a specific timeframe (e.g., in early stages of plant growth) and/or a specific tissue (e.g., an embryonic tissue, such as epicotyl, hypocotyl, radicle, and/or cotyledon). In some embodiments, the promoters of the present disclosure can be operably linked to morphogens, and can enable expression of morphogens in the seed-to-seedling developmental phase, and diminish expression during growth following the cotyledon/seedling stage, thereby producing increased numbers of normally growing transformed shoots, without the hyper-differentiation or terminal arrest as observed with constitutive expression of the morphogen. As used herein, a “seed-to-seedling developmental phase” refers to the growth period starting with germination (i.e. radicle protrusion), through emergence, continuing to the vegetative cotyledon/seedling stage, but ceasing before the first vegetative node stage. In specific embodiments, compositions and methods to express morphogens and gene editing reagents in a plant or plant part using a spatio-temporal promoter are provided. The compositions and methods provided herein can increase editing efficiency of a target gene of interest and/or generation of healthy plants or plant parts having desired edits relative to methods without morphogens or a spatio-temporal promoter. The spatio-temporal promoters disclosed herein can be used in any plant or plant parts of interest, in both monocots (e.g., maize) and dicots (e.g., legumes).

### III. Spatio-Temporal Promoters and Constructs Comprising the Same

The present disclosure provides promoter molecules for expressing polynucleotides of interest in plants or plant parts in a specific spatial, temporal, and/or spatio-temporal manner. Further, the present disclosure provides DNA constructs (e.g., expression constructs) comprising, in operable linkage, the promoter of the present disclosure and a polynucleotide of interest (e.g., encoding a morphogen, a transforming protein, a recombination modulator, a repair modulator, a defense pathway modulator, a nuclease, a selectable marker, and/or a regulatory RNA). The DNA constructs of the methods may comprise one promoter operably linked to one polynucleotide of interest. The DNA constructs may comprise more than one polynucleotides of interest, or a polynucleotide encoding more than one molecules of interest, that are operably linked to the promoter of the present disclosure. The DNA constructs may comprise one spatio-temporal promoter and operably linked to one, or more than one, polynucleotides of interest. The DNA constructs may comprise more than one promoter molecules, at least one of which is a spatio-temporal promoter, each promoter operably linked to one, or more than one, polynucleotides of interest. The DNA constructs may comprise more than one spatio-temporal promoters, each of which operably linked to one, or more than one, polynucleotides of interest. The polynucleotides or the molecules of interest can have similar types of functions (e.g., more than one morphogen, or polynucleotides encoding them) or different types of functions (e.g., a morphogen, a nuclease, and a guide RNA, or polynucleotides encoding them).

The invention encompasses isolated or substantially purified polynucleotide or nucleic acid compositions. An “isolated” or “purified” polynucleotide, or biologically active portion thereof, is substantially or essentially free from components that normally accompany or interact with the polynucleotide as found in its naturally occurring environment. Thus, an isolated or purified polynucleotide is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived.

Fragments and variants of the disclosed promoter molecules are also encompassed by the present invention. By “fragment” is intended a portion of the nucleic acid sequence. Variant sequences can be isolated by PCR as well as hybridization. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

### A. Spatio-Temporal Promoter

The present disclosure provides promoters, including 5′ untranslated regions (5′UTRs), for expression of downstream polynucleotides of interest in a plant or plant part. As used herein, “promoter” is intended to mean an upstream regulatory region of DNA prior to the ATG of a native gene, having a transcription initiation activity (e.g., function) for said gene and other downstream genes. A promoter sequence can include a 5′ untranslated region (5′UTR), including intronic sequences, in addition to a core promoter that contains a TATA box capable of directing RNA polymerase II (pol II) to initiate RNA synthesis at the appropriate transcription initiation site for a particular polynucleotide sequence of interest. A promoter may additionally comprise other recognition sequences positioned upstream of the TATA box, and well as within the 5′UTR intron, which influence the transcription initiation rate.

“Transcription initiation” as used herein refers to a phase or a process during which the first nucleotides in the RNA chain are synthesized. It is a multistep process that starts with formation of a complex between a RNA polymerase holoenzyme and a DNA template at the promoter, and ends with dissociation of the core polymerase from the promoter after the synthesis of approximately first nine nucleotides.

“Spatio-temporal promoter” as used herein refers to a promoter that is capable of initiating transcription of an operably linked polynucleotide of interest in a spatially, temporally, and/or spatio-temporally specific manner, e.g., in a tissue-specific, an axis-specific, a phase (e.g., developmental phase)-specific, a stage-specific, a timeframe-specific, and/or a timing-specific matter. “Spatio-temporal” transcription initiation as used herein refers to initiation of transcription of an operably linked polynucleotide of interest by a promoter in a spatially, temporally, and/or spatio-temporally specific manner, e.g., in a tissue-specific, an axis-specific, a phase (e.g., developmental phase)-specific, a stage-specific, a timeframe-specific, and/or a timing-specific matter. In some aspects, a spatio-temporal promoter becomes inactive (i.e., does not initiate transcription of an operably linked polynucleotide of interest) in a spatial, temporal, and/or spatio-temporal manner, e.g., outside the desired or designated tissue, axis, phase, stage, timeframe, or timing. In some aspects, a spatio-temporal promoter can turn itself on and/or off, i.e., initiate transcription in a spatial, temporal, and/or spatio-temporal manner (in a specific tissue, axis, phase, stage, timeframe, and/or timing) without exogenous regulation, and/or becomes inactive (i.e., does not initiate transcription) in a spatial, temporal, and/or spatio-temporal manner (outside a specific tissue, axis, phase, stage, timeframe, and/or timing) without exogenous regulation. Self-regulatory aspects of spatio-temporal promoters of the present disclosure, compared to inducible promoters, can help reduce the skilled labor needed to exogenously regulate activity of the polynucleotides of interest.

For instance, in some embodiments, promoter molecules of the present disclosure enable short-term, self-regulating expression of polynucleotides of interest without manual induction steps. By minimizing long-term expression, polynucleotides that can or may pose undesirable consequences e.g., in a post-germination, seedling-maturation phase, can be used or tested without compromising the regeneration process, health, or fertility of the resulting transformed plant. In some embodiments, promoter molecules of the present disclosure enable expression (e.g., initiate transcription) of an operably-linked polynucleotide of interest limited to early stages of plant growth (e.g., a seed-to-seedling developmental phase, cotyledon/seedling stage) and turn themselves off during post-cotyledon growth when the DNA construct is introduced in a plant or plant part. In some embodiments, promoter molecules of the present disclosure enable (e.g., initiate) embryonic tissue (e.g., epicotyl, hypocotyl, radicle, cotyledon, or a combination thereof)-preferred expression of an operably-linked polynucleotide of interest when the DNA construct is introduced in a plant or plant part.

The promoter molecules of the present disclosure can comprise the nucleic acid sequence for soybean XCP (e.g., Glyma.04G014800) promoter, soybean DUF1118 (e.g., Glyma.04G161600) promoter, soybean T5AH (e.g., Glyma.18G052400) promoter, pea XCP (e.g., Psat4g084640, Psat5g008960) promoter, medicago XCP (e.g., Medtr3g116080) promoter, pea DUF1118 (e.g., Psat5g207080) promoter, medicago DUF1118 (e.g., Medtr3g026020) promoter, pea T5AH (e.g., Psat5g148400) promoter, medicago T5AH (e.g., Medtr3g467130, Medtr3g467140) promoter, tomato XCP-LIKE (e.g., Solyc12g094700) promoter, Arachis hypogaea XCP-1 (e.g., arahy.Tifrunner.gnm1.ann1.8AM4UR) promoter, Arachis hypogaea XCP-2 (e.g., arahy.Tifrunner.gnm1.ann1.Q7CDUE) promoter, Cicer arietinum XCP-1 (e.g., Ca_04803) promoter, Cicer arietinum XCP-2 (e.g., Ca 17491) promoter, Lupinus albus XCP-1 (e.g., Lalb_Chr23g0265531) promoter, Lotus japonicus XCP-1 (e.g., Lj1g0003774) promoter, Phaseolus acutifolius XCP-1 (e.g., Phacu.CVR.009G145500) promoter, Phaseolus acutifolius XCP-2 (e.g., Phacu.CVR.009G145300) promoter, Phaseolus lunatus XCP-1 (e.g., P109G0000016600.v1) promoter, Phaseolus vulgaris XCP-1 (e.g., Phvul.009G008200) promoter, Phaseolus vulgaris XCP-2 (e.g., Phvul.009G008100) promoter, Trifolium pratense XCP-1 (e.g., Tp57577_TGAC_v2_gene38208) promoter, Trifolium pratense XCP-2 (e.g., Tp57577_TGAC_v2_gene15758) promoter, Vigna unguiculata XCP-1 (e.g., Vigun09g263200) promoter, Vigna unguiculata XCP-2 (e.g., Vigun09g263100) promoter, and/or fragments, variants, and combinations thereof. In some aspects, the promoter molecules of the present disclosure can comprise a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-27 and retains transcription initiation activity, or a nucleic acid sequence of any one of SEQ ID NOs: 1-27.

In some embodiments, the promoter molecules of the present disclosure further comprise a 5′UTR sequence, a 5′UTR intron sequence, an exon sequence from a coding region, and/or an intron sequence from a coding region of the sequence in the plant genome. For instance, the promoter molecules can comprise a nucleic acid sequence for soybean DUF1118 (e.g., Glyma.04G161600) exon 1, soybean DUF1118 (e.g., Glyma.04G161600) intron, pea DUF1118 (e.g., Psat5g207080) exon 1, pea DUF1118 (e.g., Psat5g207080) Intron, medicago DUF1118 (e.g., Medtr3g026020) exon 1, medicago DUF1118 (e.g., Medtr3g026020) intron, soybean T5AH (e.g., Glyma.18G052400) exon 1, soybean T5AH (e.g., Glyma.18G052400) intron, pea T5AH (e.g., Psat5g148400) exon 1, pea T5AH (e.g., Psat5g148400) intron, medicago T5AH (e.g., Medtr3g467130, Medtr3g467140) exon 1, medicago T5AH (e.g., Medtr3g467130, Medtr3g467140) intron, fragments, variants, and/or combinations thereof.

In some embodiments, the promoter molecule of the present disclosure comprises a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-3 and retains transcription initiation activity, or a nucleic acid sequence of any one of SEQ ID NOs: 1-3. The promoter molecule can initiate transcription of the operably-linked polynucleotide of interest in a spatial, temporal, and/or spatio-temporal manner. For example, the promoter molecule enables expression of the operably-linked polynucleotide of interest limited to a seed-to-seedling developmental phase when the DNA construct is introduced in a plant or plant part. In some embodiments, the promoter molecule enables embryonic tissue-preferred expression of the first polynucleotide of interest when the DNA construct is introduced in a plant or plant part. In some embodiments, the preferred embryonic tissue is epicotyl, hypocotyl, radicle, cotyledon, or a combination thereof.

The promoters of the invention can be used to express or enhance expression of any nucleic acid molecule of interest, such as any gene, polynucleotide, or regulatory element of interest. Eukaryotic promoters are complex and are comprised of components that include a TATA box consensus sequence at about 35 base pairs 5′ relative to the transcription start site or cap site which is defined as +1. The TATA motif is the site where the TATA-binding-protein (TBP) as part of a complex of several polypeptides (TFIID complex) binds and productively interacts (directly or indirectly) with factors bound to other sequence elements of the promoter. This TFIID complex in turn recruits the RNA polymerase II complex to be positioned for the start of transcription generally 25 to 30 base pairs downstream of the TATA element and promotes elongation thus producing RNA molecules. The sequences around the start of transcription (designated INR) of some pol I genes seem to provide an alternate binding site for factors that also recruit members of the TFIID complex and thus “activate” transcription. These INR sequences are particularly relevant in promoters that lack functional TATA elements providing the core promoter binding sites for eventual transcription. It has been proposed that promoters containing both a functional TATA and INR motif are the most efficient in transcriptional activity. (Zenzie-Gregory et al (1992) J. Biol. Chem. 267:2823-2830). See, for example, U.S. Pat. No. 6,072,050, herein incorporated by reference.

The spatio-temporal promoters of the invention can have a number of characteristics. For example, promoters of the present disclosure can be developmentally-regulated promoters, as discussed herein. Such promoters may show a peak in expression at a particular developmental stage. In some embodiments, the promoters of the present disclosure initiates transcription of a polynucleotide of interest limited to the seed-to-seedling developmental stage. The promoters of the present disclosure can have superior effects in expressing a polynucleotide of interest in a plant or plant part compared to developmentally-regulated promoters described in the art, e.g., U.S. Pat. No. 10,407,670; Gan and Amasino (1995) Science 270: 1986-1988; Rinehart et al. (1996) Plant Physiol 112: 1331-1341; Gray-Mitsumune et al. (1999) Plant Mol Biol 39: 657-669; Beaudoin and Rothstein (1997) Plant Mol Biol 33: 835-846; Genschik et al. (1994) Gene 148: 195-202, and the like. Additionally, the promoters of the present disclosure can express a polynucleotide of interest limited to a specific developmental stage in a self-regulatory manner, i.e., without exogenous regulation.

Additionally or alternatively, the spatio-temporal promoters of the present disclosure can be tissue-preferred promoters, as disclosed herein. In some embodiments, the promoters of the present disclosure preferably target embryonic tissue, e.g., epicotyl, hypocotyl, radicle, cotyledon, or a combination thereof. The promoters of the present disclosure can have superior effects in expressing a polynucleotide of interest in a plant or plant part compared to tissue-preferred promoters described in the art, e.g., Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Leaf-preferred promoters are also known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590. Additionally, the promoters of the present disclosure can express a polynucleotide of interest in a specific tissue in a self-regulatory manner.

Additionally or alternatively, promoters of the present disclosure can be cell-preferred promoters. Such promoters may preferentially drive the expression of a downstream polynucleotide in a particular cell type such as an embryonic tissue cell. The promoters of the present disclosure can have superior effects in expressing a polynucleotide of interest in a plant or plant part compared to cell-preferred promoters described in the art, e.g., Viret et al. (1994) Proc Natl Acad USA 91: 8577-8581; U.S. Pat. Nos. 8,455,718; 7,642,347; Sattarzadeh et al. (2010) Plant Biotechnol J 8: 112-125; Engelmann et al. (2008) Plant Physiol 146: 1773-1785; Matsuoka et al. (1994) Plant J 6: 311-319, and the like. Additionally, the promoters of the present disclosure can express a polynucleotide of interest in a specific cell in a self-regulatory manner.

Examples of promoters under developmental control include promoters that initiate transcription preferentially in certain tissues, such as leaves, roots, fruit, seeds, or flowers. A “tissue specific” promoter is a promoter that initiates transcription only in certain tissues. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the expression constructs comprise a tissue-preferred promoter. A “tissue preferred” promoter is a promoter that initiates transcription mostly, but not necessarily entirely or solely in certain tissues.

In some embodiments, the expression construct comprises a cell type specific promoter. A “cell type specific” promoter is a promoter that primarily drives expression in certain cell types in one or more organs, for example, embryonic tissue cells. The expression construct can also include cell type preferred promoters. A “cell type preferred” promoter is a promoter that primarily drives expression mostly, but not necessarily entirely or solely in certain cell types in one or more organs, for example, embryonic cells.

As disclosed herein, a specific, non-constitutive expression profile may provide an improved plant phenotype relative to constitutive expression of polynucleotides of interest. For instance, many plant genes are regulated by light conditions, the application of particular stresses, the circadian cycle, or the stage of a plant's development. These expression profiles may be important for the function of the gene or gene product in planta. One strategy that may be used to provide a desired expression profile in combination with the promoters, compositions, or methods of the present disclosure is the use of promoters containing cis-regulatory elements that drive the desired expression levels at the desired time and place in the plant. The promoters of the present disclosure can comprise cis-regulatory elements that can be used to alter polynucleotide expression in planta. Further, the promoters of the present disclosure can be superior or more efficient in expressing a polynucleotide of interest in a plant or plant part compared to promoters comprising cis-regulatory elements that have been described in the scientific literature, e.g., Vandepoele et al. (2009) Plant Physiol 150: 535-546; Rushton et al. (2002) Plant Cell 14: 749-762). Cis-regulatory elements may also be used to alter promoter expression profiles, as described in Venter (2007) Trends Plant Sci 12: 118-124.

Accordingly, the promoters of the present disclosure may produce improved effect on regeneration, development, growth, and/or physiology of plants or plant parts when expressing certain polynucleotides of interest, as compared to constitutive promoters, e.g., the CaMV 35S promoter (Odell et al. (1985) Nature 313:810-812), rice actin (McElroy et al. (1990) Plant Cell 2:163-171), ubiquitin (Christensen et al. (1989) Plant Mol. Biol. 12:619-632 and Christensen et al. (1992) Plant Mol. Biol. 18:675-689), pEMU (Last et al. (1991) Theor. Appl. Genet. 81:581-588), MAS (Velten et al. (1984) EMBO J. 3:2723-2730), ALS promoter (U.S. Pat. No. 5,659,026), and the like. Further, the promoters of the present disclosure may have improved efficiency and/or accuracy in expressing polynucleotides of interest as compared to inducible promoters known in the art, e.g., Yi et al. (2010) Planta 232: 743-754; Yamaguchi-Shinozaki and Shinozaki (1993)Mol Gen Genet 236: 331-340; U.S. Pat. No. 7,674,952; Rerksiri et al. (2013) Sci World J 2013: Article ID 397401; Khurana et al. (2013) PLoS One 8: e54418; Tao et al. (2015) Plant Mol Biol Rep 33: 200-208, and the like.

The expression levels of polynucleotides of interest can be measured by any methods known in the art. For example, polynucleotide expression levels can be measured by quantifying levels of the polynucleotide product, e.g., an RNA or a protein, by, e.g., PCR, real-time PCR, Western blotting, and ELISA. Polynucleotide expression levels can also be assessed by quantifying levels of function of polynucleotide product, for example by quantifying the occurrence of events caused by the polynucleotide product (e.g., morphology and number of regenerated shoots) or by quantifying the levels of product produced by the polynucleotide product, as further disclosed elsewhere in the present disclosure.

### B. Polynucleotide of Interest

The promoters disclosed herein can be operably linked to any polynucleotide of interest. As used herein, the term “polynucleotide of interest” can be interchangeably with the terms “coding sequence” or “nucleotide sequence of interest”. Polynucleotides of interest that are suitable for use in the present disclosed constructs include, but are not limited to, polynucleotides encoding a morphogen, a transforming protein, a recombination modulator, a repair modulator, a defense pathway modulator, a nuclease, a selectable marker, a regulatory RNA, a molecule that confers resistance to pests or disease, tolerance to herbicides, and/or advantageous agronomic traits, such as yield improvement, nitrogen use efficiency, water use efficiency, and nutritional quality. In accordance with some embodiments, the polynucleotides of interest can encode molecules that require short-term stable expression in specific tissues of interest, e.g., morphogens, modulators of recombination, repair, and defense pathways. In accordance with certain embodiments, the polynucleotides of interest can encode a selectable marker or a gene product conferring insecticidal resistance, herbicide tolerance, small RNA expression, nitrogen use efficiency, water use efficiency, or nutritional quality. Exemplary polynucleotides of interest that can be operably linked to the promoters of the present disclosure and expressed are disclosed below. More than one polynucleotides of interest, or a polynucleotide encoding more than one molecules of interest, can be operably linked to the promoter of the present disclosure. The polynucleotides or the molecules of interest can be of the same kind (e.g., more than one morphogens, or polynucleotides encoding them) or different kinds (e.g., a morphogen, a nuclease, and a guide RNA, or polynucleotides encoding them).

### 1. Morphogen

The regeneration process is a critical bottleneck in developing stably-transformed plants but can be enhanced by expressing morphogens. A “morphogen”, as used herein, refers to a molecule that is involved in organogenesis or embryogenesis. Having “morphogen activity” or “morphogenic activity”, as used herein, refers to having a function or an activity in the process of organogenesis, embryogenesis, or early development of a plant or plant part. Morphogens can be employed with, or in lieu of, exogenous phytohormones to enhance regeneration, whilst selecting for transformants using resistance markers. Morphogens more directly stimulate transformed cells to regenerate into plants. In maize transformation, advanced morphogen expression approaches have enabled more stable regenerated transformants plants being produced across and within transformed explants, with fewer inputs of skilled labor time and explant inputs, sometimes enabling transformation of recalcitrant lines. Exemplary morphogens include ISOPENTYL TRANSFERASE (IPT) and WUSCHEL 2 (WUS2), e.g., maize-derived WUS2 (ZmWUS2). Amino acid sequences for IPT and ZmWUS2 are set forth as SEQ ID NOs: 44 and 45, respectively.

In some embodiments, a polynucleotide of interest of the present disclosure can comprise a nucleic acid sequence that encodes IPT, ZmWUS2, variants, fragments, homologs, orthologs, and/or combinations thereof. In some embodiments, a polynucleotide of interest operably linked to the spatio-temporal promoter of the present disclosure encodes IPT, or its active variants or fragments. In some embodiments, a polynucleotide of interest of the present disclosure can comprise a nucleic acid sequence that encodes: (i) a polypeptide comprising an amino acid sequence having at least 80% sequence identity to SEQ ID NO: 44 and/or a polypeptide comprising an amino acid sequence having at least 80% sequence identity to SEQ ID NO: 45, wherein said polynucleotide encodes a morphogen; and/or (ii) a polypeptide comprising an amino acid sequence of SEQ ID NO: 44 or a polypeptide comprising an amino acid sequence of SEQ ID NO: 45.

In some aspects, the present disclosure provides DNA constructs comprising a first promoter molecule and a first polynucleotide of interest. The first promoter molecule can comprise a nucleic acid sequence that shares at least 80% sequence identity with any one of SEQ ID NOs: 1-27 and retains transcription initiation activity, or a nucleic acid sequence of any one of SEQ ID NOs: 1-27, as disclosed herein. In some embodiments, the first promoter molecule comprises a nucleic acid sequence that shares at least 80% sequence identity with any one of SEQ ID NOs: 1-3 and retains transcription initiation activity, or a nucleic acid sequence of any one of SEQ ID NOs: 1-3. The first polynucleotide of interest can encode one or more morphogens, e.g., IPT and/or ZmWUS2, as disclosed herein.

In some embodiments, the DNA construct of the present disclosure further comprises, in operable linkage, a second promoter molecule and a second polynucleotide of interest. The second promoter molecule can have any desirable characteristics. For example, the second promoter molecule can be a spatio-temporal promoter of the present disclosure, a promoter with a cis-regulatory element, a constitutive promoter, an inducible promoter, a tissue-specific promoter, a cell-specific promoter, a developmentally-regulated promoter, or others. In some specific embodiments, the first and second polynucleotides of interest can each encode a morphogen. In some embodiments, the first polynucleotide of interest operably linked to the first promoter molecule, i.e., a spatio-temporal promoter, comprises a nucleic acid sequence that encodes IPT, variants, fragments, and/or combinations thereof; a polypeptide comprising an amino acid sequence having at least 80% sequence identity to SEQ ID NO: 44, wherein said polynucleotide encodes a morphogen; or a polypeptide comprising an amino acid sequence of SEQ ID NO: 44; and the second polynucleotide of interest operably linked to the second promoter molecule, e.g., NOSp, comprises a nucleic acid sequence that encodes ZmWUS2, variants, fragments, and/or combinations thereof; a polypeptide comprising an amino acid sequence having at least 80% sequence identity to SEQ ID NO: 45, wherein said polynucleotide encodes a morphogen; or a polypeptide comprising an amino acid sequence of SEQ ID NO: 45. In some embodiments, the first polynucleotide of interest operably linked to the first promoter molecule, i.e., a spatio-temporal promoter, comprises a nucleic acid sequence that encodes ZmWUS2, variants, fragments, and/or combinations thereof, a polypeptide comprising an amino acid sequence having at least 80% sequence identity to SEQ ID NO: 45, wherein said polynucleotide encodes a morphogen; or a polypeptide comprising an amino acid sequence of SEQ ID NO: 45; and the second polynucleotide of interest operably linked to the second promoter molecule, e.g., 35Sp, comprises a nucleic acid sequence that encodes IPT, variants, fragments, and/or combinations thereof; a polypeptide comprising an amino acid sequence having at least 80% sequence identity to SEQ ID NO: 44, wherein said polynucleotide encodes a morphogen; or a polypeptide comprising an amino acid sequence of SEQ ID NO: 44. In some embodiments, the polynucleotide of interest comprises one morphogen, e.g., IPT or ZmWUS2, which is operably linked to the spatio-temporal promoter of the present disclosure. In some embodiments of the expression constructs disclosed herein, more than one polynucleotides of interest encoding more than one morphogen, e.g., IPT and ZmWUS2, are all operably linked to the same spatio-temporal promoter, or at least some of the morphogens are operably linked to different promoters. The promoter-polynucleotide of interest expression cassettes can be contained in the same DNA construct or separately in more than one DNA construct.

### 2. Gene Editing Reagent

A polynucleotide of interest can encode editing reagents for editing any gene or genomic site of interest. Additionally or alternatively, editing reagents for editing any gene or genomic site of interest can be introduced into a plant or plant part sequentially or simultaneously with the DNA construct provided herein. As used herein, “editing reagents” refer to a set of molecules or a construct comprising or encoding the molecules for introducing one or more mutations in the genome, including a nuclease and a guide RNA. For example, editing reagents can be CRISPR reagents, TALEN reagents, and ZFN reagents. CRISPR reagents comprise a CRISPR nuclease (e.g., Cas endonuclease or a variant thereof, such as Cas12a) and a guide RNA. In certain embodiments, the CRISPR components further comprise a tracrRNA (trans-activating CRISPR RNA) that is complementary (fully or partially) to the direct repeat sequence present on the guide RNA. A “TALEN” nuclease is an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, and yeast HO endonuclease. A “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, and yeast HO endonuclease. Editing reagents can also include base editing components. For example, cytosine base editing (CBE) reagents, which change a C-G base pair to a T-A base pair, comprise a single guide RNA, a nuclease (e.g., dCas9, CAS9 nickase), a cytidine deaminase (e.g., APOBEC1), and a uracil DNA glycosylase inhibitor (UGI). Adenine base editing (ABE) reagents, which change an A-T base pair to a G-C base pair comprise a deaminase, (TadA), a nuclease (e.g., dCas or Cas nickase), and a guide RNA.

Introducing mutations into plants or plant parts to obtain desired traits may be achieved through the use of precise genome-editing technologies to modulate the expression of the endogenous sequence. In this manner, a nucleic acid sequence can be inserted, substituted, or deleted proximal to or within a native plant sequence encoding a polynucleotide of interest through the use of methods available in the art. Such methods include, but are not limited to, use of meganucleases designed against the plant genomic sequence of interest (D'Halluin et al (2013) Plant Biotechnol J 11: 933-941); CRISPR-Cas9, CRISPR-Cas12a (Cpf1), transcription activator-like effector nucleases (TALENs), zinc finger nucleases (ZFNs), and other technologies for precise editing of genomes [Feng et al. (2013) Cell Research 23:1229-1232, Podevin et al. (2013) Trends Biotechnology 31: 375-383, Wei et al. (2013) J Gen Genomics 40:281-289, Zhang et al (2013) WO 2013/026740, Zetsche et al. (2015) Cell 163:759-771, U.S. Provisional Patent Application 62/295,325]; N. gregoryi Argonaute-mediated DNA insertion (Gao et al. (2016) Nat Biotechnol doi:10.1038/nbt.3547); Cre-lox site-specific recombination (Dale et al. (1995) Plant J 7:649-659; Lyznik, et al. (2007) Transgenic Plant J 1:1-9; FLP-FRT recombination (Li et al. (2009) Plant Physiol 151:1087-1095); Bxb1-mediated integration (Yau et al. (2011) Plant J 701:147-166); zinc-finger mediated integration (Wright et al. (2005) Plant J 44:693-705); Cai et al. (2009) Plant Mol Biol 69:699-709); and homologous recombination (Lieberman-Lazarovich and Levy (2011) Methods Mol Biol 701: 51-65; Puchta (2002) Plant Mol Biol 48:173-182).

The promoters of the present disclosure may be operably linked to a polynucleotide of interest encoding one or more nucleases. The DNA constructs of the present disclosure may comprise a polynucleotide of interest encoding one or more nucleases. Alternatively or additionally, one or more nucleases can be introduced into a plant or plant part sequentially or simultaneously with the DNA construct of the present disclosure. Nucleases can be used in the present disclosure in precise genome-editing technologies to modulate the expression of the endogenous sequence. A nuclease can be a nickase, an endonuclease, a meganuclease, or a nuclease fusion. For example, a Cas12a (Cpf1) endonuclease coupled with a guide RNA (guide RNA) designed against the genomic sequence of interest can be used (i.e., a CRISPR-Cas12a system). Alternatively, a Cas9 endonuclease coupled with a guide RNA designed against the genomic sequence of interest (a CRISPR-Cas9 system), or a Cms1 endonuclease coupled with a guide RNA designed against the genomic sequence of interest (a CRISPR-Cms1) can be used. Other nuclease systems for use with the methods of the present invention include CRISPR systems (e.g., Type I, Type II, Type III, Type IV, and/or Type V CRISPR systems (Makarova et al 2020 Nat Rev Microbiol 18:67-83)) with their corresponding guide RNA(s), TALENs, zinc finger nucleases (ZFNs), meganucleases, and the like. Alternatively, a deactivated CRISPR nuclease (e.g., a deactivated Cas9, Cas12a, or Cms1 endonuclease) fused to a transcriptional regulatory element can be targeted to the upstream regulatory region of a polynucleotide of interest, thereby modulating the function of the polynucleotide of interest (Piatek et al. (2015) Plant Biotechnol J 13:578-589). In some embodiments, the nuclease encoded by the coding sequence of the DNA construct is a CRISPR-associated Cas endonuclease. In specific embodiments, the CRISPR nuclease is a Cas12a nuclease, herein used interchangeably with a Cpf1 nuclease. In a specific embodiment, the Cas12a nuclease is a McCpf1 nuclease, e.g., a Mc.2Cpf1 2C-NLS nuclease. In some embodiments, the nuclease is further operably linked to one or more nuclear localization sequences (NLSs) and/or one or more epitope tags.

The promoters of the present disclosure may be operably linked to a polynucleotide of interest encoding one or more guide RNAs. The DNA constructs of the present disclosure may comprise a polynucleotide of interest encoding one or more guide RNAs. Alternatively or additionally, one or more guide RNAs can be introduced into a plant or plant part sequentially or simultaneously with the DNA construct of the present disclosure. To introduce one or more mutations into a gene or a genomic site of interest, antisense constructions, complementary to at least a portion of the messenger RNA (mRNA) for the sequences of the gene or the genomic site of interest can be constructed. Antisense nucleotides are designed to hybridize with the corresponding mRNA. Modifications of the antisense sequences may be made as long as the sequences hybridize to and interfere with expression of the corresponding mRNA. In this manner, antisense constructions having at least 75%, optimally 80%, more optimally 85%, 90%, 95% or greater sequence identity to the corresponding sequences to be edited may be used. Furthermore, portions of the antisense nucleotides may be used to disrupt the expression of the target gene.

In some instances, a guide RNA may comprise a targeting region that is complementary to a targeted sequence as well as another region that allows the guide RNA to form a complex with a nuclease (e.g., a CRISPR nuclease) of interest. The targeting region of a guide RNA for use in the method described herein above may be 10-40 nucleotides long (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long). For example, the targeting region of a guide RNA for use in the method described hereinabove may be 24 nucleotides in length.

In some embodiments, methods and compositions of the present disclosure can be used to introduce mutations in the genome of a plant. Editing reagents targeting any gene or genomic site of interest in a plant or plant parts can be expressed from the promoters disclosed herein. Further, the embodiments disclosed herein are not limited to certain methods of introducing nucleic acids into a plant and are not limited to certain forms or structures that the introduced nucleic acids take. Any method of transforming a cell of a plant described herein with nucleic acids are also incorporated into the teachings of this innovation, and one of ordinary skill in the art will realize that the use of particle bombardment (e.g. using a gene-gun), Agrobacterium infection and/or infection by other bacterial species capable of transferring DNA into plants (e.g., Ochrobactrum spp., Ensifer spp., Rhizobium spp.), viral infection, and other techniques can be used to deliver nucleic acid sequences into a plant described herein.

Editing reagents are not limited by certain techniques of mutagenesis. Any reagents capable of creating a change in a nucleic acid of a plant can be used in conjunction with the disclosed invention, including the use of chemical mutagens (e.g. methanesulfonate, sodium azide, aminopurine, etc.), genome/gene editing techniques (e.g. CRISPR-like technologies, TALENs, zinc finger nucleases, and meganucleases), ionizing radiation (e.g. ultraviolet and/or gamma rays) temperature alterations, long-term seed storage, tissue culture conditions, targeting induced local lesions in a genome, sequence-targeted and/or random recombinases, etc. It is anticipated that new methods of creating a mutation in a nucleic acid of a plant will be developed and yet fall within the scope of the claimed invention when used with the teachings described herein.

Any editing reagents for use in any genome-editing methods including those described herein can be operably linked to the promoter of the present disclosure and expressed in a plant or plant part.

### 3. Regulatory RNA/Small RNA

The promoters of the present disclosure may be operably linked to a polynucleotide of interest encoding regulatory RNA or small RNA. The DNA constructs of the present disclosure may comprise a polynucleotide of interest encoding regulatory RNA or small RNA. As used herein, a “regulatory RNA” refers to a non-coding RNA that regulates expression of genes. Regulatory RNAs comprise a heterogeneous group of short and long RNAs, including microRNA (miRNA) and long non-coding RNA (lncRNA). In some embodiments, the regulatory RNA for expression using the spatio-temporal promoter of the present disclosure is one or more of a microRNA (miRNA), a short-hairpin RNA, a guide RNA, a transposase, a homology-directed repair enhancer, and a non-homologous end-joining suppressor.

Various small RNA sequences can be operably linked to the promoters disclosed herein. As used herein, a “small RNA” refers to a polymeric RNA molecule, which is typically non-coding and regulates expression of genes. Types of small RNA can include microRNA (miRNA), piwi-interacting RNA (piRNA), small interfering RNA (siRNA), and small nuclear RNA 9snRNA). Examples of small RNA coding sequences that can be operably linked to the promoters of the present disclosure include delayed fruit ripening/senescence of the anti-efe small RNA delays ripening by suppressing the production of ethylene via silencing of the ACO gene that encodes an ethylene-forming enzyme. The altered lignin production of ccomt small RNA reduces content of guanacyl (G) lignin by inhibition of the endogenous S-adenosyl-L-methionine: trans-caffeoyl CoA 3-O-methyltransferase (CCOMT gene). Further, the black spot bruise tolerance in Solanum verrucosum can be reduced by the Ppo5 small RNA which triggers the degradation of Ppo5 transcripts to block black spot bruise development. Also included is the dvsnf7 small RNA that inhibits Western Corn Rootworm with dsRNA containing a 240 bp fragment of the Western Corn Rootworm Snf7 gene. Modified starch/carbohydrates can result from small RNA such as the pPhL small RNA (degrades PhL transcripts to limit the formation of reducing sugars through starch degradation) and pR1 small RNA (degrades R1 transcripts to limit the formation of reducing sugars through starch degradation). Additionally, benefits such as reduced acrylamide can result from the asnl small RNA that triggers degradation of Asnl to impair asparagine formation and reduce polyacrylamide. Finally, the non-browning phenotype of PGAS PPO suppression small RNA results in suppressing PPO to produce apples with a non-browning phenotype. The above list of small RNAs is not meant to be limiting. Any small RNA encoding sequences are encompassed by the present disclosure.

### 4. Other Polynucleotides of Interest

Various insect resistance genes can be operably linked to the promoters disclosed herein. As examples of insect resistance genes that can be operably linked to the regulatory elements of the subject disclosure, the following traits are provided. Genes that provide exemplary Lepidopteran insect resistance include: cry1A; cry1A.105; cry1Ab; cry1Ab(truncated); cry1Ab Ac (fusion protein); cry1Ac; cry1C; cry1F; cry1Fa2; cry2Ab2; cry2Ae; cry9C; mocry1F; pinII (protease inhibitor protein); vip3A(a); and vip3Aa20. Genes that provide exemplary Coleopteran insect resistance include: cry34Ab1; cry35Ab1; cry3A; cry3Bbl; dvsnf7; and mcry3A. Coding sequences that provide exemplary multi-insect resistance include ecry31.Ab. The above list of insect resistance genes is not meant to be limiting. Any insect resistance genes are encompassed by the present disclosure.

Various herbicide tolerance genes can be operably linked to the promoters disclosed herein. As examples of herbicide tolerance coding sequences that can be operably linked to the regulatory elements of the subject disclosure, the following traits are provided. The glyphosate herbicide contains a mode of action by inhibiting the EPSPS enzyme (5-enolpyruvylshikimate-3-phosphate synthase). This enzyme is involved in the biosynthesis of aromatic amino acids that are essential for growth and development of plants. Various enzymatic mechanisms are known in the art that can be utilized to inhibit this enzyme. The genes that encode such enzymes can be operably linked to any promoters disclosed herein. For example, selectable marker genes include, but are not limited to genes encoding glyphosate resistance genes such as: mutant EPSPS genes including 2mEPSPS genes, cp4 EPSPS genes, mEPSPS genes, dgt-28 genes; aroA genes; and glyphosate degradation genes such as glyphosate acetyl transferase genes (gat) and glyphosate oxidase genes (gox). Resistance genes for glufosinate and/or bialaphos compounds include dsm-2, bar and pat genes. Also included are tolerance genes that provide resistance to 2,4-D such as aad-1 genes (it should be noted that aad-1 genes have further activity on arloxyphenoxypropionate herbicides) and aad-12 genes (it should be noted that aad-12 genes have further activity on pyidyloxyacetate synthetic auxins). Resistance genes for ALS inhibitors (sulfonylureas, imidazolinones, triazolopyrimidines, pyrimidinylthiobenzoates, and sulfonylamino-carbonyl-triazolinones) are known in the art. These resistance genes most commonly result from point mutations to the ALS encoding gene sequence. Other ALS inhibitor resistance genes include hra genes, the csr1-2 genes, Sr-HrA genes, and surB genes. Herbicides that inhibit HPPD include the pyrazolones such as pyrazoxyfen, benzofenap, and topramezone; triketones such as mesotrione, sulcotrione, tembotrione, benzobicyclon; and diketonitriles such as isoxaflutole. These exemplary HPPD herbicides can be tolerated by known traits. Examples of HPPD inhibitors include hppdPF W336 genes (for resistance to isoxaflutole) and avhppd-03 genes (for resistance to meostrione). An example of oxynil herbicide tolerant traits include the bxn gene, which has been showed to impart resistance to the herbicide/antibiotic bromoxynil. Resistance genes for dicamba include the dicamba monooxygenase gene (dmo) as disclosed in International PCT Publication No. WO 2008/105890. Resistance genes for PPO or PROTOX inhibitor type herbicides (e.g., acifluorfen, butafenacil, flupropazil, pentoxazone, carfentrazone, fluazolate, pyraflufen, aclonifen, azafenidin, flumioxazin, flumiclorac, bifenox, oxyfluorfen, lactofen, fomesafen, fluoroglycofen, and sulfentrazone) are known in the art. Exemplary genes conferring resistance to PPO include over expression of a wild-type Arabidopsis thaliana PPO enzyme (Lermontova I and Grimm B, (2000) Overexpression of plastidic protoporphyrinogen IX oxidase leads to resistance to the diphenyl-ether herbicide acifluorfen. Plant Physiol 122:75-83.), the B. subtilis PPO gene (Li, X. and Nicholl D. 2005. Development of PPO inhibitor-resistant cultures and crops. Pest Manag. Sci. 61:277-285 and Choi K W, Han O, Lee H J, Yun Y C, Moon Y H, Kim M K, Kuk Y I, Han S U and Guh J O, (1998) Generation of resistance to the diphenyl ether herbicide, oxyfluorfen, via expression of the Bacillus subtilis protoporphyrinogen oxidase gene in transgenic tobacco plants. Biosci Biotechnol Biochem 62:558-560.) Resistance genes for pyri di noxy or phenoxy proprionic acids and cyclohexones include the ACCase inhibitor-encoding genes (e.g., Accl-S1, Accl-S2 and Accl-S3). Exemplary genes conferring resistance to cyclohexanedi ones and/or aryloxyphenoxypropanoic acid include haloxyfop, diclofop, fenoxyprop, fluazifop, and quizalofop. Finally, herbicides can inhibit photosynthesis, including triazine or benzonitrile are provided tolerance by psbA genes (tolerance to triazine), 1s+ genes (tolerance to triazine), and nitrilase genes (tolerance to benzonitrile). The above list of herbicide tolerance genes is not meant to be limiting. Any herbicide tolerance genes are encompassed by the present disclosure.

Various agronomic trait genes can be operably linked to the promoters disclosed herein. As examples of agronomic trait coding sequences that can be operably linked to the regulatory elements of the subject disclosure, the following traits are provided. Delayed fruit softening as provided by the pg genes inhibit the production of polygalacturonase enzyme responsible for the breakdown of pectin molecules in the cell wall, and thus causes delayed softening of the fruit. Further, delayed fruit ripening/senescence of acc genes act to suppress the normal expression of the native acc synthase gene, resulting in reduced ethylene production and delayed fruit ripening. Whereas, the accd genes metabolize the precursor of the fruit ripening hormone ethylene, resulting in delayed fruit ripening. Alternatively, the sam-k genes cause delayed ripening by reducing S-adenosylmethionine (SAM), a substrate for ethylene production. Drought stress tolerance phenotypes as provided by cspB genes maintain normal cellular functions under water stress conditions by preserving RNA stability and translation. Another example includes the EcBetA genes that catalyze the production of the osmoprotectant compound glycine betaine conferring tolerance to water stress. In addition, the RmBetA genes catalyze the production of the osmoprotectant compound glycine betaine conferring tolerance to water stress. Photosynthesis and yield enhancement is provided with the bbx32 gene that expresses a protein that interacts with one or more endogenous transcription factors to regulate the plant's day/night physiological processes. Ethanol production can be increase by expression of the amy797E genes that encode a thermostable alpha-amylase enzyme that enhances bioethanol production by increasing the thermostability of amylase used in degrading starch. Finally, modified amino acid compositions can result by the expression of the cordapA genes that encode a dihydrodipicolinate synthase enzyme that increases the production of amino acid lysine. The above list of agronomic trait coding sequences is not meant to be limiting. Any agronomic trait coding sequence is encompassed by the present disclosure.

Various selectable markers also described as reporter genes can be operably linked to the promoters disclosed herein Examples of reporter genes encode: beta-glucuronidase (GUS), luciferase, green fluorescent protein (GFP), yellow fluorescent protein (YFP, Phi-YFP), red fluorescent protein (DsRFP, RFP, etc), beta-galactosidase, and the like (See Sambrook, et al., Molecular Cloning: A Laboratory Manual, Third Edition, Cold Spring Harbor Press, N.Y., 2001, the content of which is incorporated herein by reference in its entirety).

Selectable marker genes are utilized for selection of transformed cells or tissues. Selectable marker genes include genes encoding antibiotic resistance, such as those encoding neomycin phosphotransferase II (NEO), spectinomycin/streptinomycin resistance (AAD), and hygromycin phosphotransferase (HPT or HGR) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate has been obtained by using genes coding for mutant target enzymes, 5-enolpyruvylshikimate-3-phosphate synthase (EPSPS). Genes and mutants for EPSPS are well known, and further described below. Resistance to glufosinate ammonium, bromoxynil, and 2,4-dichlorophenoxyacetate (2,4-D) have been obtained by using bacterial genes encoding PAT or DSM-2, a nitrilase, an AAD-1, or an AAD-12, each of which are examples of proteins that detoxify their respective herbicides.

Herbicides can inhibit the growing point or meristem, including imidazolinone or sulfonylurea, and genes for resistance/tolerance of acetohydroxyacid synthase (AHAS) and acetolactate synthase (ALS) for these herbicides are well known. Glyphosate resistance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include bar and pat genes from Streptomyces species, including Streptomyces hygroscopicus and Streptomyces viridichromogenes, and pyridinoxy or phenoxy proprionic acids and cyclohexones (ACCase inhibitor-encoding genes). Exemplary genes conferring resistance to cyclohexanediones and/or aryloxyphenoxypropanoic acid (including haloxyfop, diclofop, fenoxyprop, fluazifop, quizalofop) include genes of acetyl coenzyme A carboxylase (ACCase); Accl-S1, Accl-S2 and Accl-S3. Herbicides can also inhibit photosynthesis, including triazine (psbA and 1s+ genes) or benzonitrile (nitrilase gene). Further, such selectable markers can include positive selection markers such as phosphomannose isomerase (PMI) enzyme.

Selectable marker genes can further include, but are not limited to genes encoding: 2,4-D; neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA). Selectable marker genes can further include genes encoding resistance to: chloramphenicol; methotrexate; hygromycin; spectinomycin; bromoxynil; glyphosate; and phosphinothricin. The above list of selectable marker genes is not meant to be limiting. Any reporter or selectable marker gene are encompassed by the present disclosure.

The polynucleotides of interest can be synthesized for optimal expression in a plant. For example, a polynucleotide of interest can have been modified by codon optimization to enhance expression in plants. An insecticidal resistance transgene, an herbicide tolerance transgene, a nitrogen use efficiency transgene, a water use efficiency transgene, a nutritional quality transgene, a DNA binding transgene, or a selectable marker transgene/heterologous coding sequence can be optimized for expression in a particular plant species or alternatively can be modified for optimal expression in dicotyledonous or monocotyledonous plants. Plant preferred codons may be determined from the codons of highest frequency in the proteins expressed in the largest amount in the particular plant species of interest. For example, a polynucleotide of interest, e.g., a coding sequence, gene, heterologous coding sequence, or transgene/heterologous coding sequence can be designed to be expressed in plants at a higher level resulting in higher transformation efficiency. Guidance regarding the optimization and production of synthetic DNA sequences can be found in, for example, WO2013016546, WO2011146524, WO1997013402, U.S. Pat. Nos. 6,166,302, and 5,380,831, herein incorporated by reference.

### C. Other Elements of Constructs

As disclosed herein, the DNA constructs of present disclosure can comprise a first promoter molecule (a spatio-temporal promoter) operably linked to a first polynucleotide of interest, and optionally additional polynucleotide(s) of interest operably linked to the first promoter molecule, and/or additional promoter molecule(s) operably linked to additional polynucleotides of interest. In addition, the DNA constructs can comprise one or more of the following elements, and can also comprise other elements not exemplified herein.

### 1. Transfer DNA

The recombinant DNA constructs of the present disclosure may contain T-DNA sequences. For example, a recombinant DNA construct of the present disclosure may contain T-DNA of tumor-inducing (Ti) plasmid of Agrobacterium tumefaciens. Alternatively, a recombinant DNA construct of the present disclosure may contain T-DNA of tumor-inducing (Ti) plasmid of Agrobacterium rhizogenes. The vir genes of the Ti plasmid may help in transfer of T-DNA of a recombinant DNA construct into nuclear DNA genome of a host plant. For example, Ti plasmid of Agrobacterium tumefaciens may help in transfer of T-DNA of a recombinant DNA construct of the present disclosure into nuclear DNA genome of a host plant, thus enabling the transfer of a guide RNA of the present disclosure into nuclear DNA genome of a host plant (e.g., a pea plant).

### 2. Regulatory Signals

In some embodiments, a recombinant DNA construct described herein may contain additional regulatory signals, including, but not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

### 3. Reporter Genes/Selectable Marker Genes

The DNA constructs of the present disclosure may comprise reporter gene or selectable marker gene sequences. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987)Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Other selectable marker genes that could be employed on the expression constructs disclosed herein include, but are not limited to, GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449), herein incorporated by reference in their entirety.

### 4. Terminators

A transcription terminator may also be included in the expression cassettes of DNA constructs of the present invention. Plant terminators are known in the art and include those available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

### IV. Vectors, Cells, and Plants Comprising a Spatio-Temporal Promoter Construct

### A. Vectors

In some aspects, disclosed herein are vectors containing a spatio-temporal promoter of the present disclosure, or a DNA construct (e.g., a recombinant DNA construct) of the present disclosure comprising the promoter sequences of the present disclosure operably linked to a polynucleotide of interest. As used herein, “vector” refers to a nucleotide molecule (e.g., a plasmid, cosmid), bacterial phage, or virus for introducing a nucleotide construct, for example, a recombinant DNA construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide antibiotics resistance, e.g., tetracycline resistance, hygromycin resistance, or ampicillin resistance.

In some embodiments, a vector is a plasmid containing a DNA construct of the present disclosure. In some embodiments, a vector is a cosmid containing a DNA construct of the present disclosure.

In some embodiments, a vector is a recombinant virus containing a DNA construct of the present disclosure. A recombinant virus described herein can be a recombinant lentivirus, a recombinant retrovirus, a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

In some embodiments, also provided herein are expression cassettes located on a vector comprising the promoter molecule of the present disclosure operably linked to a polynucleotide of interest.

### B. Cells

In some embodiments, the present disclosure provides cells comprising a spatio-temporal promoter of the present disclosure, or a DNA construct (e.g., a recombinant DNA construct) of the present disclosure. In some embodiments, the cell is selected from the group consisting of a plant cell, a bacterial cell, and a fungal cell. For example, the present disclosure provides a bacterium, e.g., an Agrobacterium tumefaciens, containing a promoter molecule of the present disclosure or a DNA construct of the present disclosure for expressing a polynucleotide of interest, e.g., a morphogen, a transforming protein, a recombination modulator, a repair modulator, a defense pathway modulator, an editing reagent (e.g., a nuclease and/or a guide RNA) for genomic loci of interest, a selectable marker, and/or a regulatory RNA. The cells of the present disclosure may be grown, or have been grown, in a cell culture.

### C. Plants, Plant Parts, Plant Cells, and Plant Products

Disclosed herein are plants, plant parts (e.g., juice, pulp, seed, grain, fruit, flowers, nectar, embryos, pollen, ovules, leaves, stems, branches, bark, kernels, ears, cobs, husks, stalks, roots, root tips, anthers, etc.), or plant products comprising the spatio-temporal promoter molecule, the DNA construct, the vector, or the cell of the present disclosure. Also disclosed herein plants, plant parts (e.g., juice, pulp, seed, grain, fruit, flowers, nectar, embryos, pollen, ovules, leaves, stems, branches, bark, kernels, ears, cobs, husks, stalks, roots, root tips, anthers, etc.), or plant products generated by introducing the spatio-temporal promoter molecule, the DNA construct, the vector, or the cell of the present disclosure. into the plants or plant parts. “Plant products”, as used herein, refers to any product or composition produced from the plant, including any oil products, sugar products, fiber products, protein products (such as protein concentrate, protein isolate, flake, or other protein product), seed hulls, meal, or flour, for a food, feed, aqua, or industrial product, plant extract (e.g., sweetener, antioxidants, alkaloids, etc.), plant concentrate (e.g., whole plant concentrate or plant part concentrate), plant powder (e.g., formulated powder, such as formulated plant part powder (e.g., seed flour)), plant biomass (e.g., dried biomass, such as crushed and/or powdered biomass), grains, plant protein composition, plant oil composition, and food and beverage products containing plant compositions (e.g., plant parts, plant extract, plant concentrate, plant powder, plant protein, plant oil, and plant biomass) described herein. Plant parts and plant products provided herein can be intended for human or animal consumption.

A plant or plant part of the present disclosure can be a monocot. Alternatively, a plant or plant part of the present disclosure can be a dicot. A plant or plant part of the present disclosure can be a crop plant or part of a crop plant. Examples of crop plants include, but are not limited to, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally, or alternatively, a plant or plant part of the present disclosure can be a legume, i.e., a plant belonging to the family Fabaceae (or Leguminosae), or a part (e.g., fruit or seed) of such a plant. When used as a dry grain, the seed of a legume is also called a pulse. Examples of legume include, without limitation, beans (Phaseolus spp., such as tepary bean (Phaseolus acutifolius), lima bean (Phaseolus lunatus), common bean (Phaseolus vulgaris)), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Vicia faba), mung bean (Vigna radiata), lupins (Lupinus spp., such as white lupin (Lupinus albus)), mesquite (Prosopis spp.), carob (Ceratonia siliqua), tamarind (Tamarindus indica), alfalfa (Medicago sativa), Lotus japonicus, and clover (Trifolium spp.). Additionally, or alternatively, a plant or plant part of the present disclosure can be an oilseed plant (e.g., canola (Brassica napus), cotton (Gossypium spp.), camelina (Camelina sativa) and sunflower (Helianthus spp.)), or other species including wheat (Triticum spp., such as Triticum aestivum L. ssp. Aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). In some specific embodiments a plant or plant part of the present disclosure can be a dicot, e.g., a legume.

Plants or plant parts of the present disclosure can comprise a spatio-temporal promoter of the present disclosure, or a DNA construct (e.g., an expression construct) comprising, in operable linkage, the spatio-temporal promoter of the present disclosure and a polynucleotide of interest. The DNA constructs may comprise one promoter operably linked to one polynucleotide of interest. The DNA constructs may comprise more than one polynucleotides of interest, or a polynucleotide encoding more than one molecules of interest, that are operably linked to the promoter of the present disclosure. The DNA constructs may comprise one spatio-temporal promoter and operably linked to one, or more than one, polynucleotides of interest. The DNA constructs may comprise more than one promoter molecules, at least one of which is a spatio-temporal promoter, each promoter operably linked to one, or more than one, polynucleotides of interest. The DNA constructs may comprise more than one spatio-temporal promoters, each of which operably linked to one, or more than one, polynucleotides of interest. The polynucleotides or the molecules of interest can have similar types of functions (e.g., more than one morphogens, or polynucleotides encoding them) or different types of functions (e.g., a morphogen, a nuclease, and a guide RNA, or polynucleotides encoding them). The plant or plant part can comprise more than one DNA constructs each comprising different promoters and/or polynucleotides of interest. The plant or plant part can comprise more than one promoter molecules and/or more than one polynucleotides of interest.

The promoter molecules of the plants or plant parts can comprise the nucleic acid sequence for soybean XCP (e.g., Glyma.04G014800) promoter, soybean DUF1118 (e.g., Glyma.04G161600) promoter, soybean T5AH (e.g., Glyma.18G052400) promoter, pea XCP (e.g., Psat4g084640, Psat5g008960) promoter, medicago XCP (e.g., Medtr3g116080) promoter, pea DUF1118 (e.g., Psat5g207080) promoter, medicago DUF1118 (e.g., Medtr3g026020) promoter, pea T5AH (e.g., Psat5g148400) promoter, medicago T5AH (e.g., Medtr3g467130, Medtr3g467140) promoter, tomato XCP-LIKE (e.g., Solyc12g094700) promoter, Arachis hypogaea XCP-1 (e.g., arahy.Tifrunner.gnm1.ann1.8AM4UR) promoter, Arachis hypogaea XCP-2 (e.g., arahy.Tifrunner.gnm1.ann1.Q7CDUE) promoter, Cicer arietinum XCP-1 (e.g., Ca_04803) promoter, Cicer arietinum XCP-2 (e.g., Ca 17491) promoter, Lupinus albus XCP-1 (e.g., Lalb_Chr23g0265531) promoter, Lotus japonicus XCP-1 (e.g., Lj1g0003774) promoter, Phaseolus acutifolius XCP-1 (e.g., Phacu.CVR.009G145500) promoter, Phaseolus acutifolius XCP-2 (e.g., Phacu.CVR.009G145300) promoter, Phaseolus lunatus XCP-1 (e.g., P109G0000016600.v1) promoter, Phaseolus vulgaris XCP-1 (e.g., Phvul.009G008200) promoter, Phaseolus vulgaris XCP-2 (e.g., Phvul.009G008100) promoter, Trifolium pratense XCP-1 (e.g., Tp57577_TGAC_v2_gene38208) promoter, Trifolium pratense XCP-2 (e.g., Tp57577_TGAC_v2_gene15758) promoter, Vigna unguiculata XCP-1 (e.g., Vigun09g263200) promoter, Vigna unguiculata XCP-2 (e.g., Vigun09g263100) promoter, and/or fragments, variants, and combinations thereof. In some aspects, the promoter molecules of the present disclosure can comprise a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-27 and retains transcription initiation activity, or a nucleic acid sequence of any one of SEQ ID NOs: 1-27.

In some embodiments, the promoter molecules of the plants or plant parts further comprise a 5′UTR sequence, a 5′UTR intron sequence, an exon sequence from a coding region, and/or an intron sequence from a coding region of the sequence in the plant genome. For instance, the promoter molecules can comprise a nucleic acid sequence for soybean DUF1118 (e.g., Glyma.04G161600) exon 1, soybean DUF1118 (e.g., Glyma.04G161600) intron, pea DUF1118 (e.g., Psat5g207080) exon 1, pea DUF1118 (e.g., Psat5g207080) Intron, medicago DUF1118 (e.g., Medtr3g026020) exon 1, medicago DUF1118 (e.g., Medtr3g026020) intron, soybean T5AH (e.g., Glyma.18G052400) exon 1, soybean T5AH (e.g., Glyma.18G052400) intron, pea T5AH (e.g., Psat5g148400) exon 1, pea T5AH (e.g., Psat5g148400) intron, medicago T5AH (e.g., Medtr3g467130, Medtr3g467140) exon 1, medicago T5AH (e.g., Medtr3g467130, Medtr3g467140) intron, fragments, variants, and/or combinations thereof.

In some embodiments, the promoter molecule of the plant or plant part comprises a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-3 and retains transcription initiation activity, or a nucleic acid sequence of any one of SEQ ID NOs: 1-3. The promoter molecule can initiate transcription of the operably-linked polynucleotide of interest in a spatial, temporal, and/or spatio-temporal manner. For example, the promoter molecule enables expression of the operably-linked polynucleotide of interest limited to a seed-to-seedling developmental phase in a plant or plant part. In some embodiments, the promoter molecule enables embryonic tissue (e.g., epicotyl, hypocotyl, radicle, cotyledon, or a combination thereof)-preferred expression of the first polynucleotide of interest when the DNA construct is introduced in a plant or plant part.

The plants or plant parts of the present disclosure can comprise promoters operably linked to any polynucleotide of interest disclosed herein. For example, polynucleotides of interest that are suitable for use in the present disclosed constructs include, but are not limited to, polynucleotides encoding a morphogen, a transforming protein, a recombination modulator, a repair modulator, a defense pathway modulator, an editing reagent (e.g., a nuclease, a guide RNA), a selectable marker, a regulatory RNA, a small RNA, an enzyme, a transcription factor, a receptor, a ligand, a molecule that confers resistance to pests or disease, tolerance to herbicides, and/or advantageous agronomic traits (e.g., yield improvement, nitrogen use efficiency, water use efficiency, and nutritional quality). In accordance with some embodiments, the polynucleotides of interest can encode molecules that require short-term stable expression in specific tissues of interest, e.g., morphogens, modulators of recombination, repair, and defense pathways. The polynucleotides of interest can encode a selectable marker or a gene product conferring insecticidal resistance, herbicide tolerance, small RNA expression, nitrogen use efficiency, water use efficiency, or nutritional quality. More than one polynucleotides of interest, or a polynucleotide encoding more than one molecules of interest, can be operably linked to the promoter of the plant or plant part. The polynucleotides or the molecules of interest can have similar functions (e.g., more than one morphogens, or polynucleotides encoding them) or different functions (e.g., a morphogen, a nuclease, and a guide RNA, or polynucleotides encoding them).

In some specific embodiments, a polynucleotide of interest of the plant or plant part can comprise a nucleic acid sequence that encodes IPT, ZmWUS2, variants, fragments, and/or combinations thereof. In some embodiments, a polynucleotide of interest operably linked to the spatio-temporal promoter of the present disclosure is IPT, or its variants or fragments. In some embodiments, a polynucleotide of interest of the present disclosure can comprise a nucleic acid sequence that encodes: (i) a polypeptide comprising an amino acid sequence having at least 80% sequence identity to SEQ ID NO: 44 and/or a polypeptide comprising an amino acid sequence having at least 80% sequence identity to SEQ ID NO: 45, wherein said polynucleotide encodes a morphogen; and/or (ii) a polypeptide comprising an amino acid sequence of SEQ ID NO: 44 or a polypeptide comprising an amino acid sequence of SEQ ID NO: 45.

The plants or plant parts can comprise DNA constructs comprising a first promoter molecule and a first polynucleotide of interest. The first promoter molecule can comprise a spatio-promoter of the present disclosure. The first polynucleotide of interest can encode one or more morphogens, e.g., IPT and/or ZmWUS2, as disclosed herein.

In some embodiments, the DNA construct of the plant or plant part further comprises a second promoter molecule operably linked to a second polynucleotide of interest. The second promoter molecule can have any desirable characteristics. For example, the second promoter molecule can be a spatio-temporal promoter of the present disclosure, a promoter with a cis-regulatory element, a constitutive promoter, an inducible promoter, a tissue-specific promoter, a cell-specific promoter, a developmentally-regulated promoter, or others. In some specific embodiments, the first and second polynucleotides of interest can each encode a morphogen. In some embodiments, the first polynucleotide of interest operably linked to the first promoter molecule, i.e., a spatio-temporal promoter, comprises a nucleic acid sequence that encodes IPT, variants, fragments, and/or combinations thereof; a polypeptide comprising an amino acid sequence having at least 80% sequence identity to SEQ ID NO: 44, wherein said polynucleotide encodes a morphogen; or a polypeptide comprising an amino acid sequence of SEQ ID NO: 44; and the second polynucleotide of interest operably linked to the second promoter molecule, e.g., NOSp, comprises a nucleic acid sequence that encodes ZmWUS2, variants, fragments, and/or combinations thereof; a polypeptide comprising an amino acid sequence having at least 80% sequence identity to SEQ ID NO: 45, wherein said polynucleotide encodes a morphogen; or a polypeptide comprising an amino acid sequence of SEQ ID NO: 45. In some embodiments, the first polynucleotide of interest operably linked to the first promoter molecule, i.e., a spatio-temporal promoter, comprises a nucleic acid sequence that encodes ZmWUS2, variants, fragments, and/or combinations thereof, a polypeptide comprising an amino acid sequence having at least 80% sequence identity to SEQ ID NO: 45, wherein said polynucleotide encodes a morphogen; or a polypeptide comprising an amino acid sequence of SEQ ID NO: 45; and the second polynucleotide of interest operably linked to the second promoter molecule, e.g., 35Sp, comprises a nucleic acid sequence that encodes IPT, variants, fragments, and/or combinations thereof; a polypeptide comprising an amino acid sequence having at least 80% sequence identity to SEQ ID NO: 44, wherein said polynucleotide encodes a morphogen; or a polypeptide comprising an amino acid sequence of SEQ ID NO: 44. In some embodiments, the polynucleotide of interest comprises one morphogen, e.g., IPT or ZmWUS2, which is operably linked to the spatio-temporal promoter of the present disclosure. In some embodiments, the polynucleotides of interest comprise more than one morphogens, e.g., IPT and ZmWUS2, wherein the morphogens are all operably linked to the same spatio-temporal promoter, or at least some of the morphogens are operably linked to different promoters. The promoter-polynucleotide of interest cassettes can be contained in the same DNA construct or separately in more than one DNA construct.

In some embodiments, the promoter molecule(s) and/or the polynucleotide(s) of interest are stably inserted in the genome of said plant or plant part. In particular embodiments, the promoter molecule(s) and/or the polynucleotide(s) of interest are transiently expressed in the plant or plant part and/or are not integrated into the plant genome.

In some embodiments, the polynucleotide of interest operably linked to the spatio-temporal promoter is expressed in a specific spatial, temporal, and/or spatio-temporal manner. In some embodiments, the polynucleotide of interest, operably linked to a promoter that does not have a spatio-temporal function can be constitutively expressed; expressed throughout (i.e., ubiquitous expression); expressed more strongly in certain tissues or cells, e.g., embryonic tissues or cells, compared to other tissues or cells; in a developmentally-regulated manner; or expressed upon induction via an inducible promoter, in the plant or plant part.

Also provided herein are plant parts (e.g., juice, pulp, seed, grain, fruit, flowers, nectar, embryos, pollen, ovules, leaves, stems, branches, bark, kernels, ears, cobs, husks, stalks, roots, root tips, anthers, etc.), plant extract (e.g., protein, sweetener, antioxidants, alkaloids, etc.), plant concentrate (e.g., whole plant concentrate, plant part concentrate, or protein concentrate), plant powder [e.g., formulated powder, such as formulated plant part powder (e.g., seed flour)], and plant biomass (e.g., dried biomass, such as crushed and/or powdered biomass) obtained from plants of the present disclosure. Also provided herein are seeds, such as a representative sample of seeds, from a plant of the present disclosure.

Molecules encoded by the DNA constructs of the present disclosure (e.g., promoters, polynucleotides of interest) may be found in plants or plant parts to which the DNA constructs have been introduced, or plants or plant parts regenerated therefrom according to the methods of the present disclosure. Mutations introduced by the methods using the DNA constructs encoding editing reagents may be found in plants or plant parts to which the DNA constructs have been introduced, or plants or plant parts regenerated therefrom according to the methods of the present disclosure. Mutations can also be found in plant parts, plant extract, plant concentrate, plant powder, and plant biomass obtained from such plants.

Also provided herein are food and/or beverage products containing plant compositions (e.g., plant parts, plant extract, plant concentrate, plant powder, plant protein, and plant biomass) described hereinabove, such as plant compositions derived from the plants or plant parts of the present disclosure. Such food and/or beverage products include, without limitation, shakes, juices, health drinks, alternative meat products (e.g., meatless burger patties, meatless sausages, etc.), alternative egg products (e.g., eggless mayo), and non-dairy products (e.g., non-dairy whipped toppings, non-dairy milk, non-dairy creamer, non-dairy milk shakes, etc, and condiments. A food and/or beverage product that contains plant compositions obtained from plants or plant parts of the present disclosure can have desired traits, compared to a similar or comparable food and/or beverage product that contains plant compositions obtained from a control plant or plant part.

Plant parts (e.g., seeds) and plant products (e.g., plant biomass, seed compositions, protein compositions, food and/or beverage products) produced by the methods provided herein can be meant for consumption by agricultural animals or for use as feed in an agriculture or aquaculture system. In specific embodiments, plant parts and plant products produced according to the methods provided herein include animal feed (e.g., roughages—forage, hay, silage; concentrates—cereal grains, soybean cake) intended for consumption by bovine, porcine, poultry, lambs, goats, or any other agricultural animal. In some embodiments, plant parts and plant products produced according to the methods include aquaculture feed for any type of fish or aquatic animal in a farmed or wild environment including, without limitation, trout, carp, catfish, salmon, tilapia, crab, lobster, shrimp, oysters, clams, mussels, and scallops.

Where the invention is described in terms of transformed plants, it is recognized that transformed organisms of the invention also include plant cells, plant protoplasts, plant cell tissue cultures from which plants can be regenerated, plant calli, plant clumps, and plant cells that are intact in plants or parts of plants such as embryos, pollen, ovules, seeds, grains, leaves, flowers, branches, fruit, kernels, ears, cobs, husks, stalks, roots, root tips, anthers, and the like. Progeny, variants, and mutants of the regenerated plants are also included within the scope of the invention, provided that these parts comprise the introduced polynucleotides.

### V. Methods of Expressing a Polynucleotide of Interest in a Plant

Disclosed herein are methods of expressing a nucleotide sequence of interest in a plant or plant part (e.g., juice, pulp, seed, fruit, flower, nectar, embryo, pollen, ovule, leaf, stem, branch, bark, kernel, ear, cob, husk, stalk, root, root tip, anther) by introducing into the plant or the plant part the promoter molecule or the DNA construct of the present disclosure. Also disclosed herein are methods of transforming a plant or plant part by introducing into the plant or the plant part the promoter molecule or the DNA construct of the present disclosure and regenerating a transformed plant or plant part from said plant cell. In some embodiments, the promoter molecule or the DNA construct is introduced into the plant or the plant part by stable transformation. In other embodiments, the promoter molecule or the DNA construct is introduced into the plant by transient transformation.

### A. Transformation of Plants

Provided herein are methods for transforming plants or plant parts by introducing into the plants or plant parts a construct for expressing a polynucleotide of interest or for introducing one or more mutations (e.g., insertions, substitutions, or deletions) at a desired target site in the plant genome, wherein the construct comprises a promoter disclosed herein. The term “transform” or “transformation” refers to any method used to introduce polypeptides or polynucleotides into plant cells. For purpose of the present disclosure, the transformation can be: “stable transformation”, wherein the transformation construct (e.g., a construct comprising a polynucleotide of interest encoding a morphogen, a transforming protein, a recombination modulator, a repair modulator, a defense pathway modulator, an editing reagent (e.g., a nuclease, a guide RNA), a selectable marker, a regulatory RNA, a small RNA, an enzyme, a transcription factor, a receptor, a ligand, a molecule that confers resistance to pests or disease, tolerance to herbicides, and/or advantageous agronomic traits, for use in the methods of the present invention) is introduced into a host (e.g., a host plant, plant part, plant cell, etc.) and integrates into the genome of the host and is capable of being inherited by the progeny thereof, or “transient transformation”, wherein the transformation construct is introduced into a host (e.g., a host plant, plant part, plant cell, etc.) and expressed temporarily. The methods disclosed herein can also be used for insertion of heterologous polynucleotides and/or modification of native plant gene expression to achieve desirable plant traits, e.g., increased sugar content.

The promoters disclosed herein and/or any polynucleotide of interest operably linked to a promoter disclosed herein can be introduced into a plant cell, organelle, or plant embryo by a variety of means of transformation, including microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration [see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens)]; all of which are herein incorporated by reference.

Agrobacterium-and biolistic-mediated transformation remain the two predominantly employed approaches. However, transformation may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, viral infection, Agrobacterium and viral mediated (Caulimoriviruses, Geminiviruses, RNA plant viruses), liposome mediated and the like. Methods disclosed herein are not limited to any size of nucleic acid sequences that are introduced, and thus one could introduce a nucleic acid comprising a single nucleotide (e.g. an insertion) into a nucleic acid of the plant and still be within the teachings described herein. Nucleic acids introduced in substantially any useful form, for example, on supernumerary chromosomes (e.g. B chromosomes), plasmids, vector constructs, additional genomic chromosomes (e.g. substitution lines), and other forms is also anticipated. It is envisioned that new methods of introducing nucleic acids into plants and new forms or structures of nucleic acids will be discovered and yet fall within the scope of the claimed invention when used with the teachings described herein.

More than one polynucleotides of interest (e.g., a morphogen, a transforming protein, a recombination modulator, a repair modulator, a defense pathway modulator, an editing reagent (e.g., a nuclease, a guide RNA), a selectable marker, a regulatory RNA, a small RNA, an enzyme, a transcription factor, a receptor, a ligand, a molecule that confers resistance to pests or disease, tolerance to herbicides, and/or advantageous agronomic traits(e.g., yield improvement, nitrogen use efficiency, water use efficiency, and nutritional quality) can be introduced into the plant, plant cell, plant organelle, or plant embryo simultaneously or sequentially. More than one polynucleotides of interest can be introduced into the plant, plant cell, plant organelle, or plant embryo by introducing one DNA construct that comprise all the polynucleotides of interest operably linked to one or more promoters. Alternatively, more than one polynucleotides of interest can be introduced into the plant, plant cell, plant organelle, or plant embryo by introducing more than one DNA constructs that each comprise some of the polynucleotides of interest operably linked to one or more promoters simultaneously or sequentially. For example, a morphogen (or morphogens) and editing reagents can be introduced into the plant, plant cell, plant organelle, or plant embryo in one DNA construct, or in more than one DNA construct simultaneously or sequentially. The amount or ratio of more than one polynucleotides of interest, or molecules encoded therein, can be adjusted by adjusting the amount or concentration of the polynucleotides and/or timing and dosage of introducing the polynucleotides into the plant or plant part. In specific embodiments, polynucleotides of interest encode nuclease and guide RNA(s), and the ratio of the nuclease (or encoding nucleic acid) to the guide RNA(s) (or encoding DNA) generally will be about stoichiometric such that the two components can form an RNA-protein complex with the target DNA.

The cells that have been transformed may be cultured and grown into plants in accordance with conventional ways. See, for example, McCormick et al. (1986) Plant Cell Reports 5:81-84. In this manner, the present invention provides transformed plants or plant parts, transformed seed (also referred to as “transgenic seed”) or transformed plant progenies having a nucleic acid modification stably incorporated into their genome.

The present invention may be used for transformation of any plant species, e.g., both monocots and dicots. The present invention can be used for transformation of crop plants or part of crop plants, e.g., corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally or alternatively, the present invention can be used for transformation of a legume, i.e., a plant belonging to the family Fabaceae (or Leguminosae), or a part (e.g., fruit or seed) of such a plant, e.g., beans (Phaseolus spp., such as tepary bean (Phaseolus acutifolius), lima bean (Phaseolus lunatus), common bean (Phaseolus vulgaris)), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Vicia faba), mung bean (Vigna radiata), lupins (Lupinus spp., such as white lupin (Lupinus albus)), mesquite (Prosopis spp.), carob (Ceratonia siliqua), tamarind (Tamarindus indica), alfalfa (Medicago sativa), Lotus japonicus, and clover (Trifolium spp.). Additionally or alternatively, the present invention can be used for transformation of an oilseed plant (e.g., canola (Brassica napus), cotton (Gossypium spp.), camelina (Camelina sativa) and sunflower (Helianthus spp.)), or other species including wheat (Triticum spp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). In specific embodiments, the present invention can be used for transformation of dicots, e.g., legumes.

Also disclosed herein are plants and plant parts generated by the methods of the present disclosure, and plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant concentrate (e.g., whole plant concentrate or plant part concentrate), plant powder [e.g., formulated powder, such as formulated plant part powder (e.g., seed flour)], and plant biomass (e.g., dried biomass, such as crushed and/or powdered biomass), and food or beverage products obtained from plants of the present disclosure. Also provided herein are seeds, such as a representative sample of seeds, from a plant generated by the methods of the present disclosure.

### B. Confirmation of Transformation

A transformed plant cell, callus, tissue or plant may be identified and isolated by selecting or screening the engineered plant material for traits encoded by the marker genes present on the transforming DNA. For instance, selection can be performed by growing the engineered plant material on media containing an inhibitory amount of the antibiotic or herbicide to which the transforming gene construct confers resistance. Further, transformed plants and plant cells can also be identified by screening for the activities of any visible marker genes (e.g., the 3-glucuronidase, luciferase, or green fluorescent protein genes) that may be present on the recombinant nucleic acid constructs. Such selection and screening methodologies are well known to those skilled in the art. Molecular confirmation methods that can be used to identify transgenic plants are known to those with skill in the art. Several exemplary methods are further described below.

Molecular Beacons have been described for use in sequence detection. Briefly, a FRET oligonucleotide probe is designed that overlaps the flanking genomic and insert DNA junction. The unique structure of the FRET probe results in it containing a secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe(s) to the target sequence results in the removal of the probe secondary structure and spatial separation of the fluorescent and quenching moieties. A fluorescent signal indicates the presence of the flanking genomic/transgene insert sequence due to successful amplification and hybridization. Such a molecular beacon assay for detection of as an amplification reaction is an embodiment of the subject disclosure.

Hydrolysis probe assay is a method of detecting and quantifying the presence of a DNA sequence. Briefly, a FRET oligonucleotide probe is designed with one oligo within the transgene/heterologous coding sequence and one in the flanking genomic sequence for event-specific detection. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization. Such a hydrolysis probe assay for detection of as an amplification reaction is an embodiment of the subject disclosure.

A method of detecting and quantifying the presence of a DNA sequence by detecting an amplification reaction can be used. Briefly, the genomic DNA sample comprising the integrated gene expression cassette polynucleotide is screened using a polymerase chain reaction (PCR) based assay. The assay can utilize a PCR assay mixture which contains multiple primers. The primers used in the PCR assay mixture can comprise at least one forward primers and at least one reverse primer. The forward primer contains a sequence corresponding to a specific region of the DNA polynucleotide, and the reverse primer contains a sequence corresponding to a specific region of the genomic sequence. In addition, the primers used in the PCR assay mixture can comprise at least one forward primers and at least one reverse primer. For example, the PCR assay mixture can use two forward primers corresponding to two different alleles and one reverse primer. One of the forward primers contains a sequence corresponding to specific region of the endogenous genomic sequence. The second forward primer contains a sequence corresponding to a specific region of the DNA polynucleotide. The reverse primer contains a sequence corresponding to a specific region of the genomic sequence.

In some embodiments the fluorescent signal or fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye.

In other embodiments the amplification reaction is run using suitable second fluorescent DNA dyes that are capable of staining cellular DNA at a concentration range detectable by flow cytometry, and have a fluorescent emission spectrum which is detectable by a real time thermocycler. It should be appreciated by those of ordinary skill in the art that other nucleic acid dyes are known and are continually being identified. Any suitable nucleic acid dye with appropriate excitation and emission spectra can be employed.

In further embodiments, Next Generation Sequencing (NGS) can be used for detection. As described by Brautigma et al., 2010, DNA sequence analysis can be used to determine the nucleotide sequence of the isolated and amplified fragment. The amplified fragments can be isolated and sub-cloned into a vector and sequenced using chain-terminator method (also referred to as Sanger sequencing) or Dye-terminator sequencing. In addition, the amplicon can be sequenced with Next Generation Sequencing. NGS technologies do not require the sub-cloning step, and multiple sequencing reads can be completed in a single reaction.

The confirmation methods include a long read NGS, which uses emulsion PCR and pyrosequencing to generate sequencing reads. DNA fragments of 300-800 bp or libraries containing fragments of 3-20 kb can be used. The reactions can produce over a million reads of about 250 to 400 bases per run for a total yield of 250 to 400 megabases. This technology produces the longest reads but the total sequence output per run is low compared to other NGS technologies.

The confirmation methods also include is a short read NGS which uses sequencing by synthesis approach with fluorescent dye-labeled reversible terminator nucleotides and is based on solid-phase bridge PCR. Construction of paired end sequencing libraries containing DNA fragments of up to 10 kb can be used. The reactions produce over 100 million short reads that are 35-76 bases in length. This data can produce from 3-6 gigabases per run.

The confirmation methods also include a short read technology that uses fragmented double stranded DNA that are up to 10 kb in length. The system uses sequencing by ligation of dye-labelled oligonucleotide primers and emulsion PCR to generate one billion short reads that result in a total sequence output of up to 30 gigabases per run.

A NGS approach can use single DNA molecules for the sequence reactions, e.g., by producing up to 800 million short reads that result in 21 gigabases per run. These reactions are completed using fluorescent dye-labelled virtual terminator nucleotides that is described as a “sequencing by synthesis” approach. A NGS approach can also use a real time sequencing by synthesis. This technology can produce reads of up to 1,000 bp in length as a result of not being limited by reversible terminators. Raw read throughput that is equivalent to one-fold coverage of a diploid human genome can be produced per day using this technology.

In another embodiment, the detection can be completed using blotting assays, including Western blots, Northern blots, and Southern blots. Such blotting assays are commonly used techniques in biological research for the identification and quantification of biological samples. These assays include first separating the sample components in gels by electrophoresis, followed by transfer of the electrophoretically separated components from the gels to transfer membranes that are made of materials such as nitrocellulose, polyvinylidene fluoride (PVDF), or Nylon. Analytes can also be directly spotted on these supports or directed to specific regions on the supports by applying vacuum, capillary action, or pressure, without prior separation. The transfer membranes are then commonly subjected to a post-transfer treatment to enhance the ability of the analytes to be distinguished from each other and detected, either visually or by automated readers.

In a further embodiment the detection can be completed using an ELISA assay, which uses a solid-phase enzyme immunoassay to detect the presence of a substance, usually an antigen, in a liquid sample or wet sample. Antigens from the sample are attached to a surface of a plate. Then, a further specific antibody is applied over the surface so it can bind to the antigen. This antibody is linked to an enzyme, and, in the final step, a substance containing the enzyme's substrate is added. The subsequent reaction produces a detectable signal, most commonly a color change in the substrate.

### C. Expressing a Polynucleotide of Interest in a Plant

In some aspects, provided herein is a method of expressing a polynucleotide of interest in a plant or plant part comprising introducing a DNA construct into said plant or plant part, wherein the DNA construct comprises, in operable linkage a first promoter and a first polynucleotide of interest. In some aspects, provided herein is a method of transforming a plant or plant part by introducing a DNA construct, comprising a first promoter operably linked to a first polynucleotide of interest into a plant cell, and regenerating a transformed plant or plant part from said plant cell. The DNA constructs of the methods may comprise one promoter operably linked to one polynucleotide of interest. The DNA constructs of the methods may comprise more than one polynucleotides of interest, or a polynucleotide encoding more than one molecules of interest, that are operably linked to the promoter of the present disclosure. The DNA constructs may comprise one spatio-temporal promoter and operably linked to one, or more than one, polynucleotides of interest. The DNA constructs may comprise more than one promoter molecules, at least one of which is a spatio-temporal promoter, each promoter operably linked to one, or more than one, polynucleotides of interest. The DNA constructs may comprise more than one spatio-temporal promoters, each of which operably linked to one, or more than one, polynucleotides of interest. The polynucleotides or the molecules of interest can have similar types of functions (e.g., more than one morphogens, or polynucleotides encoding them) or different types of functions (e.g., a morphogen, a nuclease, and a guide RNA, or polynucleotides encoding them).

The method of the present disclosure can comprise introducing into a plant, plant part, or plant cell a promoter molecule operably linked to a polynucleotide of interest. The method can also comprise introducing into a plant, plant part, or plant cell more than one polynucleotides of interest simultaneously or sequentially. More than one polynucleotides of interest can be introduced into the plant, plant part, or plant cell by introducing one DNA construct that comprise all the polynucleotides of interest operably linked to one or more promoters. Alternatively, more than one polynucleotides of interest can be introduced into the plant, plant part, or plant cell by introducing more than one DNA constructs that each comprise some of the polynucleotides of interest operably linked to one or more promoters simultaneously or sequentially.

The DNA construct (e.g., an expression construct) according to the methods of the present disclosure can comprise a spatio-temporal promoter of the present disclosure. The promoter molecules of the methods can comprise the nucleic acid sequence for soybean XCP (e.g., Glyma.04G014800) promoter, soybean DUF1118 (e.g., Glyma.04G161600) promoter, soybean T5AH (e.g., Glyma.18G052400) promoter, pea XCP (e.g., Psat4g084640, Psat5g008960) promoter, medicago XCP (e.g., Medtr3g116080) promoter, pea DUF1118 (e.g., Psat5g207080) promoter, medicago DUF1118 (e.g., Medtr3g026020) promoter, pea T5AH (e.g., Psat5g148400) promoter, medicago T5AH (e.g., Medtr3g467130, Medtr3g467140) promoter, tomato XCP-LIKE (e.g., Solyc12g094700) promoter, Arachis hypogaea XCP-1 (e.g., arahy..gnm1 ann1.8AM4UR) promoter, Arachis hypogaea XCP-2 (e.g., arahy.Tifrunner.gnm1.ann1.Q7CDUE) promoter, Cicer arietinum XCP-1 (e.g., Ca_Tifrunner 04803) promoter, Cicer arietinum XCP-2 (e.g., Ca_17491) promoter, Lupinus albus XCP-1 (e.g., Lalb_Chr23g0265531) promoter, Lotus japonicus XCP-1 (e.g., Lj1g0003774) promoter, Phaseolus acutifolius XCP-1 (e.g., Phacu.CVR.009G145500) promoter, Phaseolus acutifolius XCP-2 (e.g., Phacu.CVR.009G145300) promoter, Phaseolus lunatus XCP-1 (e.g., P109G0000016600.v1) promoter, Phaseolus vulgaris XCP-1 (e.g., Phvul.009G008200) promoter, Phaseolus vulgaris XCP-2 (e.g., Phvul.009G008100) promoter, Trifolium pratense XCP-1 (e.g., Tp57577_TGAC_v2_gene38208) promoter, Trifolium pratense XCP-2 (e.g., Tp57577_TGAC_v2_gene15758) promoter, Vigna unguiculata XCP-1 (e.g., Vigun09g263200) promoter, Vigna unguiculata XCP-2 (e.g., Vigun09g263100) promoter, and/or fragments, variants, and combinations thereof. In some aspects, the promoter molecules of the methods for expressing a polynucleotide of interest in a plant or plant part, or the methods for transforming a plant or plant part, can comprise a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-27 and retains transcription initiation activity, or a nucleic acid sequence of any one of SEQ ID NOs: 1-27. In some embodiments, the promoter molecule of the methods comprises a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-3 and retains transcription initiation activity, or a nucleic acid sequence of any one of SEQ ID NOs: 1-3.

In some embodiments, the promoter molecules of the methods further comprise a 5′UTR sequence, a 5′UTR intron sequence, an exon sequence from a coding region, and/or an intron sequence from a coding region of the sequence in the plant genome. For instance, the promoter molecules can comprise a nucleic acid sequence for soybean DUF1118 (e.g., Glyma.04G161600) exon 1, soybean DUF1118 (e.g., Glyma.04G161600) intron, pea DUF1118 (e.g., Psat5g207080) exon 1, pea DUF1118 (e.g., Psat5g207080) Intron, medicago DUF1118 (e.g., Medtr3g026020) exon 1, medicago DUF1118 (e.g., Medtr3g026020) intron, soybean T5AH (e.g., Glyma.18G052400) exon 1, soybean T5AH (e.g., Glyma.18G052400) intron, pea T5AH (e.g., Psat5g148400) exon 1, pea T5AH (e.g., Psat5g148400) intron, medicago T5AH (e.g., Medtr3g467130, Medtr3g467140) exon 1, medicago T5AH (e.g., Medtr3g467130, Medtr3g467140) intron, fragments, variants, and/or combinations thereof.

The promoter molecule of the methods can initiate transcription of the operably-linked polynucleotide of interest in a spatial, temporal, and/or spatio-temporal manner. For example, in some specific embodiments, the promoter molecule enables expression of the operably-linked polynucleotide of interest limited to a seed-to-seedling developmental phase in a plant or plant part. In other specific embodiments, the promoter molecule enables embryonic tissue (e.g., epicotyl, hypocotyl, radicle, cotyledon, or a combination thereof)-preferred expression of the first polynucleotide of interest in a plant or plant part. The promoter molecule of the methods can exert its transcription initiation function in a self-regulated manner without exogenous regulation. For example, the promoter of the methods can turn itself off outside the specific tissue (e.g., embryonic tissue) or the specific phase, stage, timeframe, or timing (e.g., after seed-to-seedling developmental phase).

In some embodiments, expression or function of polynucleotide(s) of interest within the desired or designated tissue, axis, phase, stage, timeframe, or timing can be greater by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more compared to the expression or function of the polynucleotide(s) of interest outside the desired or designated tissue, axis, phase, stage, timeframe, or timing. In some embodiments, the expression or function of the polynucleotide(s) of interest within the desired or designated tissue, axis, phase, stage, timeframe, or timing can be greater by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45% 50%, 55%, 60%, 65%, 70%, 75%, 80%, 85%, 90%, 95%, 100%, 200%, 300%, 400%, 500%, 600%, 700%, 800%, 900%, 1000%, or more, or by at least 10%, 15%, 20%, 25%, 30%, 35%, 40%, 45% 50%, 55%, 60%, 65%, 70%, 75%, 80%, 85%, 90%, 95%, 100%, 200%, 300%, 400%, 500%, 600%, 700%, 800%, 900%, 1000%, or more compared to the baseline expression or function of the polynucleotide(s) of interest in a plant or plant part without the polynucleotide(s) of interest being introduced. In some embodiments, the expression or function of the polynucleotide(s) of interest outside the desired or designated tissue, axis, phase, stage, timeframe, or timing is not increased compared to the baseline expression or function of the polynucleotide(s) of interest in a plant or plant part without the polynucleotide(s) of interest being introduced. In some embodiments, the expression or function of the polynucleotide(s) of interest outside the desired or designated tissue, axis, phase, stage, timeframe, or timing is reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or by at least 10%, 15%, 20%, 25%, 30%, 35%, 40%, 45%, 50%, 55%, 60%, 65%, 70%, 75%, 80%, 85%, 90%, 95%, or 100%, as compared to the expression or function of the polynucleotide(s) of interest operably linked to a spatio-temporal promoter of the present disclosure within the desired or designated tissue, axis, phase, stage, timeframe, or timing; or as compared to the expression or as compared to the expression or function of the polynucleotide(s) of interest operably linked to control promoter.

In specific embodiments the polynucleotides of interest are genes encoding a protein. Gene or polynucleotide expression levels can be measured by any methods known in the art. For example, gene or polynucleotide expression levels can be measured by quantifying levels of the gene or polynucleotide product, e.g., an RNA or a protein, by, e.g., PCR, real-time PCR, Western blotting, and ELISA. Gene or polynucleotide expression levels can also be assessed by quantifying levels of function of gene or polynucleotide product, for example by quantifying the occurrence of events caused by the gene or polynucleotide product (e.g., shoot regeneration) or by quantifying the levels of product produced by the gene or polynucleotide product.

The methods of the present disclosure can be used to express any polynucleotide of interest disclosed herein in a plant or plant part. The methods of the present disclosure can be used to transform a plant or plant part with any polynucleotide of interest. For example, polynucleotides of interest that are suitable for use in the present disclosed constructs include, but are not limited to, polynucleotides encoding a morphogen, a transforming protein, a recombination modulator, a repair modulator, a defense pathway modulator, an editing reagent (e.g., a nuclease, a guide RNA), a selectable marker, a regulatory RNA, a small RNA, an enzyme, a transcription factor, a receptor, a ligand, a molecule that confers resistance to pests or disease, tolerance to herbicides, and/or advantageous agronomic traits (e.g., yield improvement, nitrogen use efficiency, water use efficiency, and nutritional quality). The polynucleotides of interest can encode molecules that require short-term stable expression in specific tissues of interest, e.g., morphogens, modulators of recombination, repair, and defense pathways. The polynucleotides of interest can encode a selectable marker or a gene product conferring insecticidal resistance, herbicide tolerance, small RNA expression, nitrogen use efficiency, water use efficiency, or nutritional quality. More than one polynucleotides of interest, or a polynucleotide encoding more than one molecules of interest, can be operably linked to the promoter of the plant or plant part. The polynucleotides or the molecules of interest can have similar functions (e.g., more than one morphogens, or polynucleotides encoding them) or different functions (e.g., a morphogen, a nuclease, and a guide RNA, or polynucleotides encoding them).

In some specific embodiments, a polynucleotide of interest of the plant or plant part can comprise a nucleic acid sequence that encodes IPT, ZmWUS2, variants, fragments, and/or combinations thereof. In some embodiments, a polynucleotide of interest operably linked to the spatio-temporal promoter of the present disclosure encodes IPT, or its variants or fragments. In some embodiments, a polynucleotide of interest of the present disclosure can comprise a nucleic acid sequence that encodes: a polypeptide comprising an amino acid sequence having at least 80% sequence identity to SEQ ID NO: 44, wherein said polynucleotide encodes a morphogen; a polypeptide comprising an amino acid sequence having at least 80% sequence identity to SEQ ID NO: 45, wherein said polynucleotide encodes a morphogen; a polypeptide comprising an amino acid sequence of SEQ ID NO: 44; and/or a polypeptide comprising an amino acid sequence of SEQ ID NO: 45.

DNA constructs of the methods can comprise a first promoter molecule and a first polynucleotide of interest. The first promoter molecule can comprise a spatio-promoter of the present disclosure. The first polynucleotide of interest can encode one or more morphogens, e.g., IPT, ZmWUS2, and/or variants, fragments, combinations thereof, as disclosed herein. In specific embodiments, the first polynucleotide of interest encodes IPT or its variants or fragments.

In some embodiments, the DNA construct of the method further comprises a second promoter molecule operably linked to a second polynucleotide of interest. The second promoter molecule can have any desirable characteristics. For example, the second promoter molecule can be a spatio-temporal promoter of the present disclosure, a promoter with a cis-regulatory element, a constitutive promoter, an inducible promoter, a tissue-specific promoter, a cell-specific promoter, a developmentally-regulated promoter, or others. In some specific embodiments, the first and second polynucleotides of interest each encode a morphogen. In some embodiments, the first polynucleotide of interest operably linked to the first promoter molecule, i.e., a spatio-temporal promoter, comprises a nucleic acid sequence that encodes IPT, variants, fragments, and/or combinations thereof, a polypeptide comprising an amino acid sequence having at least 80% sequence identity to SEQ ID NO: 44, wherein said polynucleotide encodes a morphogen; or a polypeptide comprising an amino acid sequence of SEQ ID NO: 44. In some embodiments, the second polynucleotide of interest operably linked to the second promoter molecule, e.g., NOSp, comprises a nucleic acid sequence that encodes ZmWUS2, variants, fragments, and/or combinations thereof; a polypeptide comprising an amino acid sequence having at least 80% sequence identity to SEQ ID NO: 45, wherein said polynucleotide encodes a morphogen; or a polypeptide comprising an amino acid sequence of SEQ ID NO: 45. In some embodiments, the first polynucleotide of interest operably linked to the first promoter molecule, i.e., a spatio-temporal promoter, comprises a nucleic acid sequence that encodes ZmWUS2, variants, fragments, and/or combinations thereof, a polypeptide comprising an amino acid sequence having at least 80% sequence identity to SEQ ID NO: 45, wherein said polynucleotide encodes a morphogen; or a polypeptide comprising an amino acid sequence of SEQ ID NO: 45. In some embodiments, the second polynucleotide of interest operably linked to the second promoter molecule, e.g., 35Sp, comprises a nucleic acid sequence that encodes IPT, variants, fragments, and/or combinations thereof, a polypeptide comprising an amino acid sequence having at least 80% sequence identity to SEQ ID NO: 44, wherein said polynucleotide encodes a morphogen; or a polypeptide comprising an amino acid sequence of SEQ ID NO: 44. In some embodiments, the polynucleotide of interest comprises one morphogen, e.g., IPT or ZmWUS2, which is operably linked to the spatio-temporal promoter of the present disclosure. In some embodiments, the polynucleotides of interest comprise more than one morphogens, e.g., IPT and ZmWUS2, wherein the morphogens are all operably linked to the same spatio-temporal promoter, or at least some of the morphogens are operably linked to different promoters.

Accordingly, in some embodiments, IPT (or its variants or fragments) is operably linked to a spatio-temporal promoter of the present disclosure, and is introduced into a plant, plant part, or plant cell. In some embodiments, IPT and ZmWUS2 (or their variants or fragments) are operably linked to one spatio-temporal promoter or two separate promoters, at least one of which is a spatio-temporal promoter, and introduced into a plant, plant part, or plant cell in one DNA construct, or in individual DNA constructs, simultaneously or sequentially.

In some embodiments, the method further comprises introducing additional DNA constructs, e.g., a second DNA construct, more than one additional DNA constructs, into said plant or plant part, simultaneously or sequentially with a first DNA construct comprising a spatio-temporal promoter and polynucleotides of interest. The second DNA construct or the additional DNA constructs can comprise a promoter operably linked to a polynucleotide of interest. The promoter of the second DNA construct or additional DNA constructs can have any desirable characteristics. For example, the second promoter molecule can be a spatio-temporal promoter of the present disclosure, a promoter with a cis-regulatory element, a constitutive promoter, an inducible promoter, a tissue-specific promoter, a cell-specific promoter, a developmentally-regulated promoter, or others. In some embodiments, polynucleotides of interest are introduced into a plant, plant part, or plant cell using more than one DNA constructs. For example, a morphogen and editing reagents can be introduced into a plant, plant part, or plant cell using more than one DNA constructs.

In some embodiments, the method comprises introducing one or more morphogens and editing reagents into a plant or plant part using a spatio-temporal promoter and/or a DNA construct provided herein, to increase editing efficiency of a target gene of interest and/or generation of healthy plants or plant parts having desired edits relative to methods without morphogens or a spatio-temporal promoter. One or more morphogens and editing reagents can be introduced into a plant, plant part, or plant cell using one or more DNA constructs. In specific embodiments, a DNA construct encoding morphogens and editing reagents, each operably linked to a promoter, are introduced into a plant, plant part, or plant cell. For example, a construct comprising (i) a promoter molecule and one or more operably linked polynucleotides encoding one or more guide RNAs, (ii) a promoter molecule and an operably linked polynucleotide encoding a nuclease, (iii) a promoter molecule (e.g., a spatio-temporal promoter) and an operably linked polynucleotide that encodes IPT (e.g., a polypeptide comprising an amino acid sequence that shares at least 80% sequence identity with SEQ ID NO: 44, wherein said polypeptide has morphogenic activity, or a polypeptide comprising an amino acid sequence of SEQ ID NO: 44), and (iv) a promoter molecule (e.g., a constitutive promoter or a spatio-temporal promoter) and an operably linked polynucleotide that encodes ZmWUS2 (e.g., a polypeptide comprising an amino acid sequence that shares at least 80% sequence identity with SEQ ID NO: 45, wherein said polypeptide has morphogenic activity, or a polypeptide comprising an amino acid sequence of SEQ ID NO: 45) is introduced into a plant, plant part, or plant cell for efficient editing of a target gene and/or high recovery of transformants with desired edits.

In some embodiments, the promoter molecule(s) and/or the polynucleotide(s) of interest are stably inserted in the genome of said plant or plant part. In particular embodiments, the promoter molecule(s) and/or the polynucleotide(s) of interest are transiently expressed in the plant or plant part and/or are not integrated into the plant genome.

In some embodiments, the polynucleotide of interest operably linked to the spatio-temporal promoter is expressed in a specific spatial, temporal, and/or spatio-temporal manner. In some embodiments, the polynucleotide of interest, operably linked to a promoter that does not have a spatio-temporal function can be constitutively expressed; expressed throughout (i.e., ubiquitous expression); expressed more strongly in certain tissues or cells, e.g., embryonic tissues or cells, compared to other tissues or cells; in a developmentally-regulated manner; or expressed upon induction via an inducible promoter, in the plant or plant part.

In some embodiments of the methods of the present disclosure, the polynucleotide of interest is stably inserted into a genome of said plant or plant part. In some embodiments, the polynucleotide of interest is transiently expressed in said plant or plant part.

In some embodiments of the methods of present disclosure, the plant is selected from the group consisting of corn (Zea mays), Brassica species, Brassica napus, Brassica rapa, Brassica juncea, alfalfa (Medicago sativa), pea (Pisum sativum), fava bean (Vicia faba), common bean (Phaseolus vulgaris), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. In some embodiments, said plant is a dicot. In some embodiments, said plant is a legume.

The present disclosure provides plants or plant parts produced by the method of the present disclosure, wherein said plant or plant part comprises said DNA construct.

D. Enhancing Viability and/or Production of Transformed Plants

In some aspects, the present disclosure provides a method of transforming a plant or plant part by introducing a DNA construct, comprising a first promoter operably linked to a first polynucleotide of interest into a plant cell, and regenerating a transformed plant or plant part from said plant cell. The first promoter can be a spatio-temporal promoter, and can allow short-term, stable expression of a polynucleotide of interest in specific tissues of interest. In some embodiments of the methods of present disclosure, the promoter molecule initiates expression of the polynucleotide limited to a seed-to-seedling developmental phase in the plant or plant part. In some embodiments of the methods of present disclosure, the promoter molecule initiates embryonic tissue-preferred expression of the polynucleotide in the plant or plant part. In some embodiments, wherein the preferred embryonic tissue is epicotyl, hypocotyl, radicle, cotyledon, or a combination thereof.

Such spatio-temporal expression of a polynucleotide of interest can have superior effect on regeneration, development, growth, and/or physiology of plants or plant parts compared to expressing the polynucleotide of interest in a plant or plant part using a control promoter (e.g., a constitutive promoter), particularly when expressing a polynucleotide of interest whose constitutive or unregulated expression is toxic or is inconsistent with endogenous physiological expression patterns. Effect on regeneration, development, growth, and/or physiology of plants or plant parts can be assessed by methods known in the art, including analyzing the morphology of the plant or plant part, the size of the plant or plant part, the color of the plant or plant part, the number or frequency of germination or shoots, or metabolites in the plant or plant part.

In some embodiments, the method of the present disclosure increases normal shoot formation, a frequency of shoot producing plants or plant parts, and/or a number of regenerated shoots from transformed plants or plant parts relative to a control method comprising introducing a control DNA construct comprising a control promoter molecule into a plant cell. In some embodiments, normal shoot formation, a frequency of shoot producing plants or plant parts, and/or a number of regenerated shoots from transformed plants or plant parts are increased by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more with methods using a promoter of the present disclosure, as compared to using a control promoter. Normal shoot formation, a frequency of shoot producing plants or plant parts, and/or a number of regenerated shoots from transformed plants or plant parts can be increased by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more according to the methods using the spatio-temporal promoter of the present disclosure compared to the control method using a control promoter. As used herein, a “control promoter” is a promoter that is not capable of initiating transcription of an operably linked polynucleotide of interest in a spatially, temporally, and/or spatio-temporally specific manner in a plant or plant part. A control promoter can be a promoter that does not comprise a nucleic acid sequence that shares at least 80% sequence identity with any one of SEQ ID NOs: 1-27 and retains transcription initiation activity, or a nucleic acid sequence of any one of SEQ ID NOs: 1-27. As used herein, a control promoter can be a constitutive or ubiquitious promoter, or any other promoter, useful for determining the effect of the spatio-temporal promoters disclosed herein. In some embodiments, the frequency of shoot producing plants or plant parts according to the methods is increased by about 10% to about 500% relative to the control method. In some embodiments, the number of regenerated shoots from transformed plants or plant parts according to the methods is increased by about 10% to about 1200% relative to the control method. Normal shoot formation, a frequency of shoot producing plants or plant parts, and/or a number of regenerated shoots from transformed plants or plant parts can be analyzed by observing plants according to methods and protocols known in the art.

### E. Breeding of Plants

Disclosed herein are methods for breeding a plant, such as a plant comprising a promoter molecule and/or a DNA construct of the present disclosure, or a plant generated according to the methods of the present disclosure. A plant containing the one or more heterogeneous nucleic acid sequences of the present disclosure may be regenerated from a plant cell or plant part, wherein the genome of the plant cell or plant part is genetically-modified to contain the one or more mutations or the polynucleotide of the present disclosure. Using conventional breeding techniques or self-pollination, one or more seeds may be produced from the plant that contains the one or more mutations or the polynucleotide of the present disclosure. Such a seed, and the resulting progeny plant grown from such a seed, may contain the one or more mutations or the polynucleotide of the present disclosure, and therefore may be transgenic. Progeny plants are plants having a genetic modification to contain the one or more mutations or the polynucleotide of the present disclosure, which descended from the original plant having modification to contain the one or more mutations or the polynucleotide of the present disclosure. Seeds produced using such a plant of the invention can be harvested and used to grow generations of plants having genetic modification to contain the one or more mutations or the polynucleotide of the present disclosure, e.g., progeny plants, of the invention, comprising the polynucleotide and optionally expressing a polynucleotide of agronomic interest (e.g., herbicide resistance gene). Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

### EXAMPLES

The following examples are offered by way of illustration and not by way of limitation. All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

### Example 1: Diagrams of Exemplary Spatio-Temporal Promoters

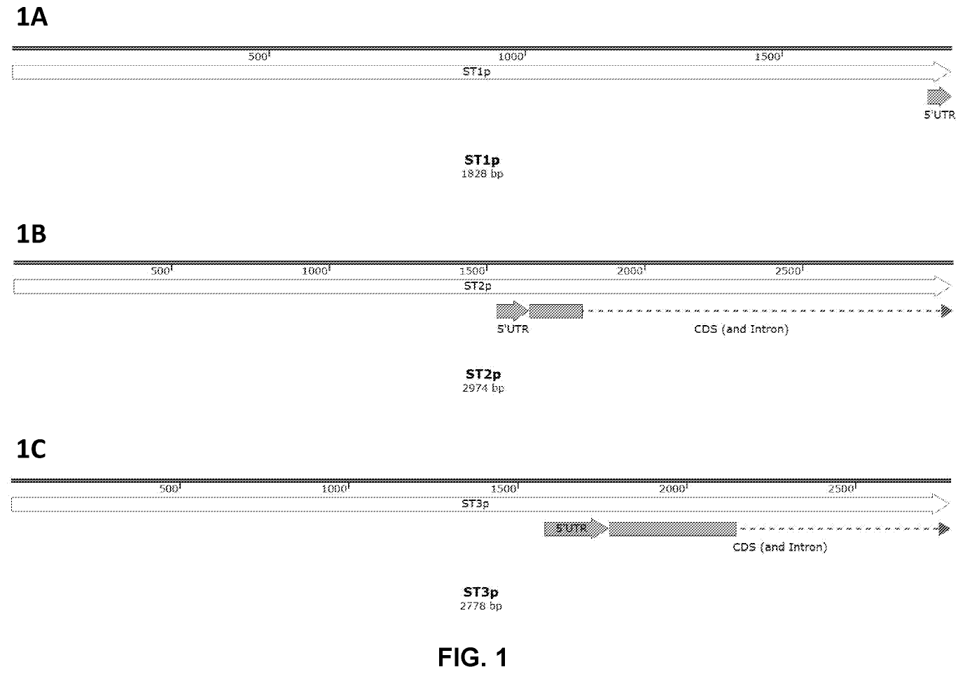

Sequence diagrams of exemplary spatio-temporal promoters, ST1p, ST2p, and ST3p are respectively depicted as items A, B, and C in FIG. 1. The top row depicts the nucleic acid sequence of the Spatio-Temporal 1 promoter (ST1p), also called TN1p, corresponding to a sequence from −1783 bases to +45 bases from the transcriptional start site (TSS) of xylem cysteine proteinase (XCP, Glyma.04G014800) in the soybean genome. The second row depicts the nucleic acid sequence of the Spatio-Temporal 2 promoter (ST2p), also called LM1p, corresponding to a sequence from −1531 bases to +1443 bases from the TSS of protein of unknown function DUF1118 (Glyma.04G161600) in the soybean genome. The third row depicts the nucleic acid sequence of the Spatio-Temporal 3 promoter (ST3p), also called RNHp, corresponding to a sequence from −1580 to +1198 bases from the TSS of taxadiene 5-alpha-hydroxylase (T5AH, Glyma.18G052400) in the soybean genome. The nucleic acid sequences of ST1p, ST2p, and ST3p are set forth as SEQ ID NOs: 1, 2, and 3, respectively.

### Example 2: Validation of Spatio-Temporal Promoters for Morphogen Expression in Stable Regenerative Yellow Pea Transformation

ST1p, ST2p, and ST3p were individually fused to the polynucleotides encoding the ISOPENTYL TRANSFERASE (IPT) and Zea mays WUSCHEL 2 (ZmWUS2) morphogen gene. The amino acid sequences of IPT and ZmWUS2 are set forth as SEQ ID NOs: 44 and 45, respectively. Each spatio-temporal morphogen cassette was cloned into a T-DNA vector. In some experiments, the spatio-temporal morphogen cassette (encoding IPT or ZmWUS2) was cloned into a T-DNA vector along with the other morphogen (ZmWUS2 or IPT) operably linked to a constitutive promoter, for which 35Sp operably linked to IPT (35Sp-IPT) and NOSp operably linked to ZmWUS2 (NOSp-ZmWUS2) were used. These constructs conferred spectinomycin resistance and were used to transform Agrobacterium tumefaciens AGL-1 strain. These constructs were tested for impact on shoots elicited in pea (Pisum sativum ‘Variety 1’) using an Agrobacterium-mediated embryonic meristem transformation system. Explant inputs were 100 for the negative control (no morphogens, with an ER-localized GFP transformation reporter), and 200 for morphogen treatments.

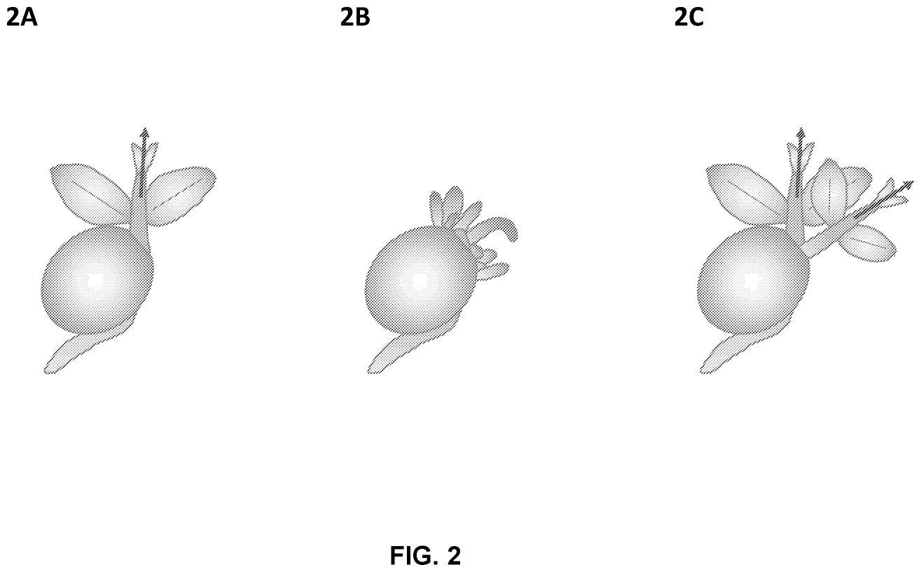

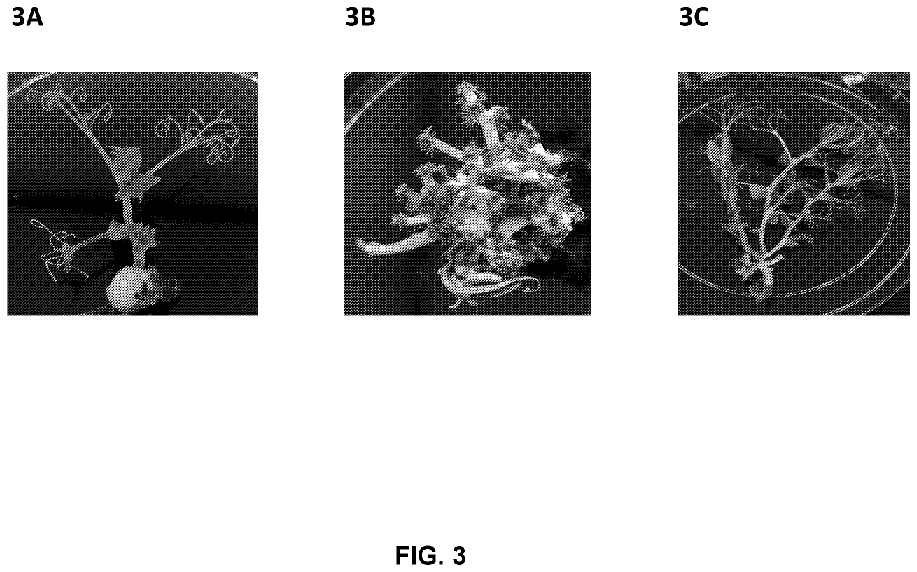

Transformation responses of explants were recorded as “shoot”, “morphogenic”, or “multiple shoots”, which are schematically depicted in FIG. 2. FIG. 2A represents formation of a normal shoot (“shoot”). FIG. 2B represents formation of multiple immature shoots that are not transplantable (“morphogenic”). FIG. 2C represents formation of multiple normal shoots (“multiple shoots”). FIG. 3A is a representative image of a pea transformant that has produced a normal shoot (“shoot”) following delivery of a negative control construct (no morphogens). FIG. 3B is a representative image of a pea transformant that has produced multiple immature shoots that are not transplantable (“morphogenic”) following delivery of the 35Sp_IPT_NOSp_ZmWUS2 morphogens construct. FIG. 3C is a representative image of a pea transformant that has produced multiple normal shoots (“multiple shoots”) following delivery of the ST1p_IPT_NOSp_ZmWUS2 morphogens construct. A greater number of phenotypically normal shoots were produced by the pea transformant expressing the IPT morphogen under the ST1p promoter disclosed herein, than by either of the other transformants (FIG. 3C vs. FIG. 3A, 3B).

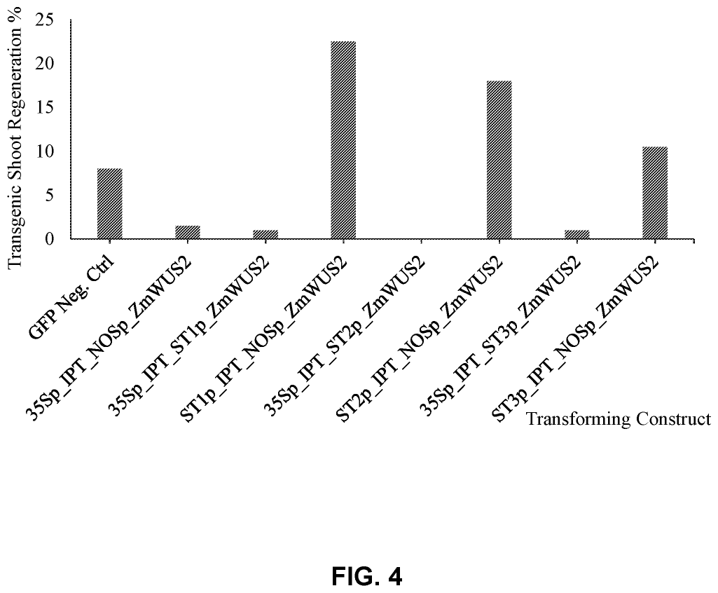

As shown in FIG. 4, the 35Sp_IPT_NOSp_ZmWUS2 (constitutive) construct elicited a hyper-differentiation/morphogenic response in shoots, which were not regenerable and were not counted as positive in shooting quantifications. On the other hand, the ST1p_IPT_NOSp_ZmWUS2 construct elicited a significant (p-value=0.002, Fisher's exact test) three-times increase over the negative control (no morphogens) in the percentage of transformed explants that regenerated normally growing transgenic shoots with respect to the total transformed explants (shooting %). Similarly, the ST2p_IPT_NOSp_ZmWUS2 construct produced a significant (p-value=0.024, Fisher's exact test) two-times increase over the negative control (no morphogens) in the shooting %. Moreover, as some explants produced multiple transgenic shoots, the total number of transgenic regenerated shoots was 12-times higher for the ST1p_IPT_NOSp_ZmWUS2 construct than the negative control. Additionally, the total number of transgenic regenerated shoots was 7-times higher for the ST2p_IPT_NOSp_ZmWUS2 construct than the negative control. The aforementioned data is shown in more detail in Table 1, with construct names simplified to emphasize the morphogen gene fused to a spatio-temporal promoter.

TABLE 1
Effects of Trailing Promoters for Morphogen Expression on Shooting Responses in T0 Pea Plants

| | | No. of | | Explants | % Shooting (Explants | | |
|---|---|---|---|---|---|---|---|
| | Total | explants | % Explants | producing | producing shoots/total | Total no. | |
| Construct | explants | responding | responding | shoots | explants) | of shoots | Response |
| GFP Neg. Ctrl | 100 | 8 | 8 | 8 | 8 | 8 | Shoot |
| Constitutive | 200 | 49 | 24.5 | 3 | 1.5 | 3 | Morphogenic |
| ST1p-ZmWUS2 | 200 | 54 | 27 | 2 | 1 | 2 | Morphogenic |
| ST1p-IPT | 200 | 45 | 22.5 | 45 | 22.1 | 99 | Multiple shoots |
| ST2p-ZmWUS2 | 200 | 28 | 14 | 0 | 0 | 0 | Morphogenic |
| ST2p-IPT | 200 | 36 | 17.6 | 36 | 17.6 | 53 | Multiple shoots |
| ST3p-ZmWUS2 | 200 | 40 | 20 | 2 | 1 | 2 | Morphogenic |
| ST3p-IPT | 200 | 21 | 10.3 | 21 | 10.3 | 21 | Shoot |

As for the constructs in which a spatio-temporal promoter was operably linked to ZmWUS2 and a constitutive promoter (35Sp) was operably linked to IPT, there were no increases in shoot regeneration for any combination of spatio-temporal promoter for ZmWUS2 expression with constitutive IPT expression (35Sp_IPT).

A follow-up experiment was conducted with the ST1p_IPT_NOSp_ZmWUS2 and 35Sp_IPT_NOSp_ZmWUS2 constructs, as well as the negative control, using 200 explant inputs for the negative control and morphogen treatments.

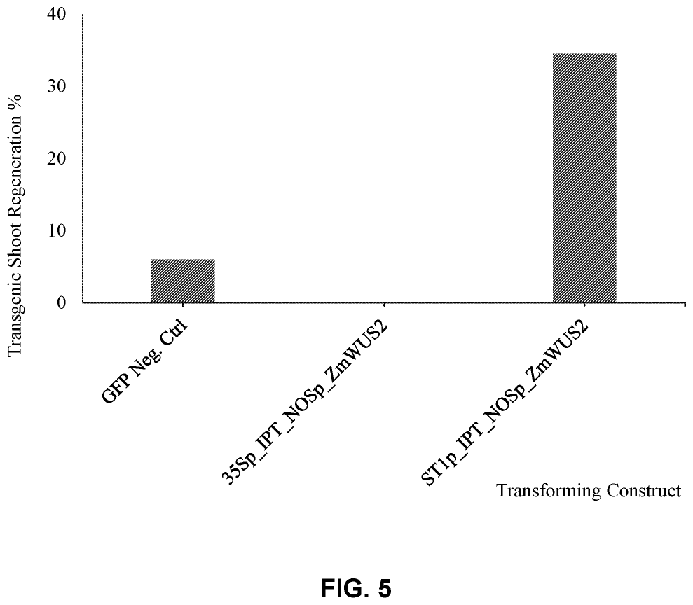

As shown in FIG. 5, the ST1p_IPT_NOSp_ZmWUS2 construct elicited a significant (p-value=<0.00001, Fisher's exact test) 6-times increase over the negative control in the shooting %. As before, some explants produced multiple transgenic shoots when transformed with the ST1p_IPT_NOSp_ZmWUS2 construct, so the total number of transgenic regenerated shoots was 13-times higher for the ST1p_IPT_NOSp_ZmWUS2 construct than the negative control. Again, the 35Sp_IPT_NOSp_ZmWUS2 construct resulted in a decrease (to zero) in shooting % compared to the negative control.

TABLE 2
Effects of Lead Promoters for Morphogen Expression on Shooting Responses in T0 Pea Plants

| | | No. of | | Explants | % Shooting (Explants | | |
|---|---|---|---|---|---|---|---|
| | Total | explants | % Explants | producing | regenerating transgenic | Total no. | |
| Construct | explants | responding | responding | shoots | shoots/total explants) | of shoots | Response |
| GFP Neg. Ctrl | 200 | 13 | 6.5 | 12 | 6 | 12 | Shoot |
| Constitutive | 200 | 64 | 32 | 0 | 0 | 0 | Morphogenic |
| ST1p-IPT | 200 | 74 | 37 | 69 | 34.5 | 160 | Multiple shoots |

### Example 3: Validation of Spatio-Temporal Promoters for Morphogen Expression in Stable Regenerative Soybean Transformation

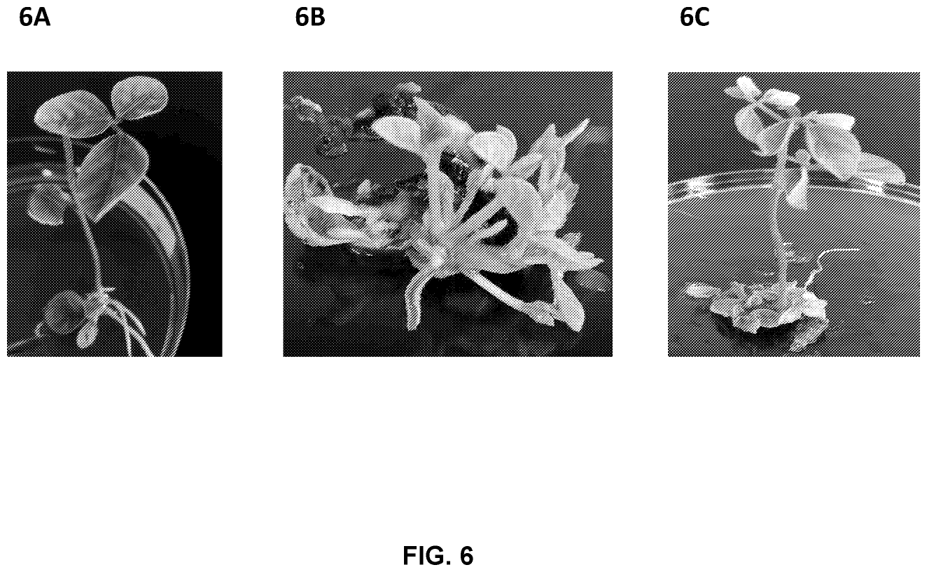

The spatio-temporal morphogen constructs, constitutive morphogen constructs, and negative control, tested in pea in Example 2 were tested in soybean (Glycine max ‘Variety 1’). Using an Agrobacterium-mediated embryonic meristem transformation system, 100 explants were transformed with the negative control, and 200 explants were transformed with morphogen constructs. FIG. 6A is a representative image of a soybean transformant that has produced a normal shoot (“shoot”) following delivery of a negative control construct (no morphogens). FIG. 6B is a representative image of a soybean transformant that has produced multiple immature shoots that are not transplantable (“morphogenic”) following delivery of the 35Sp_IPT_NOSp_ZmWUS2 morphogens construct. FIG. 6C is a representative image of a soybean transformant that has produced a normal primary shoot (“shoot”) following delivery of the ST1p_IPT_NOSp_ZmWUS2 morphogens construct.

As shown in Table 3, introducing ST1p_IPT or ST3p_IPT into the soybean embryos resulted in production of shoots with a normal phenotype.

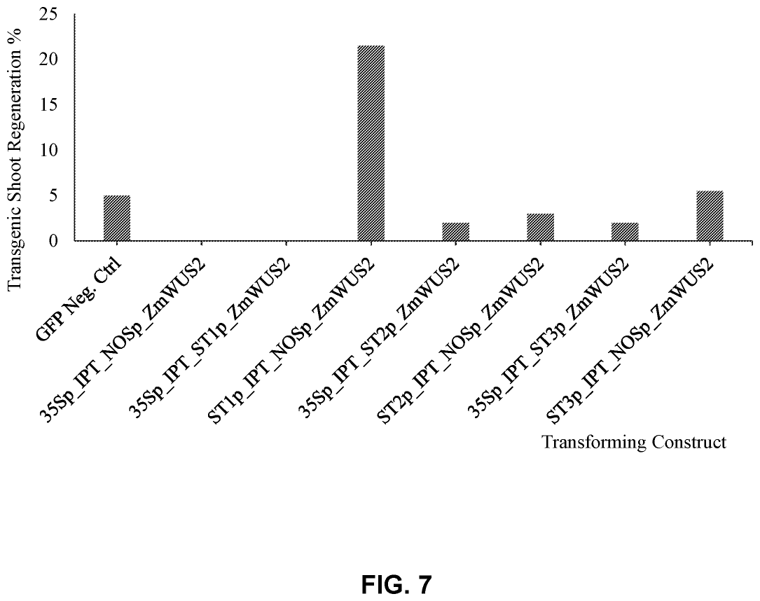

As shown in FIG. 7, the use of ST1p_IPT_NOSp_ZmWUS2 elicited a significant (p-value=0.0002, Fisher's exact test) four-times increase over the negative control (no morphogens) in the percentage of transformed explants that regenerated normally growing transgenic shoots with respect to the total transformed explants (shooting %). Typically only one transgenic shoot was regenerated per transformed explant in soybean, which differed from the “multiple shoots” regenerated per explant in pea. As before in pea, the 35Sp_IPT_NOSp_ZmWUS2 (constitutive) construct elicited a hyper-differentiation/morphogenic response in shoots, which were not regenerable and were not counted as positive in shooting quantifications. On the other hand, the ST2p_IPT_NOSp_ZmWUS2 construct elicited regeneration-compromised (morphogenic) shoots similar to those seen with constitutively-expressed morphogens, even though the same ST2p_IPT_NOSp_ZmWUS2 construct elicited production of normal shoots in pea. The aforementioned data is shown in more detail in Table 3, with construct names simplified to emphasize the morphogen gene fused to a spatio-temporal promoter.

TABLE 3
Effects of Trialing Promoters for Morphogen Expression
on Shooting Responses in T0 Soybean Plants

| | | No. of | | Explants | % Shooting (Explants | |
|---|---|---|---|---|---|---|
| | Total | explants | % Explants | producing | regenerating transgenic | |
| Construct | explants | responding | responding | shoots | shoots/total explants) | Response |
| GFP Neg. Ctrl | 100 | 5 | 5 | 5 | 5 | Shoot |
| Constitutive | 200 | 52 | 26 | 0 | 0 | Morphogenic |
| ST1p-ZmWUS2 | 200 | 17 | 8.5 | 0 | 0 | Morphogenic |
| ST1p-IPT | 200 | 43 | 21.5 | 43 | 21.5 | Shoot |
| ST2p-ZmWUS2 | 200 | 21 | 10.5 | 4 | 2 | Morphogenic |
| ST2p-IPT | 200 | 9 | 4.5 | 6 | 3 | Morphogenic |
| ST3p-ZmWUS2 | 200 | 37 | 18.5 | 4 | 2 | Morphogenic |
| ST3p-IPT | 200 | 11 | 5.5 | 11 | 5.5 | Shoot |

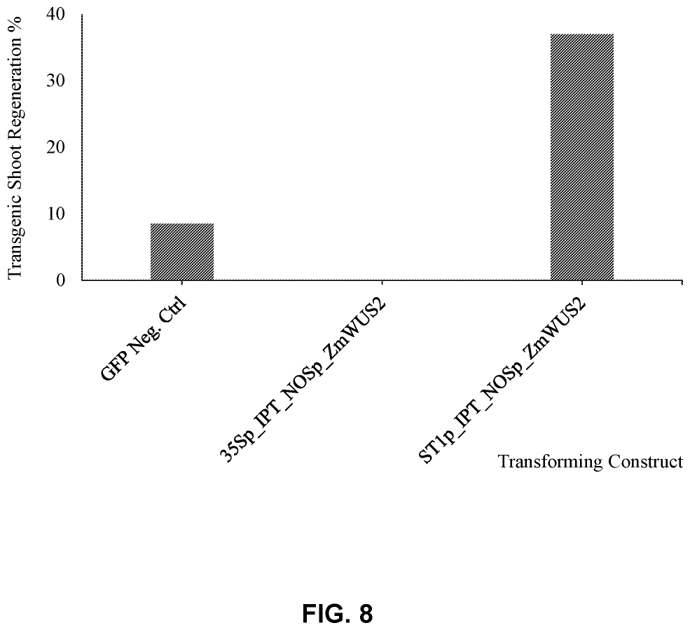

A follow up experiment was conducted in soybean (Glycine max ‘Variety 1’) with the ST1p_IPT_NOSp_ZmWUS2 and 35Sp_IPT_NOSp_ZmWUS2 constructs, as well as the negative control, using 200 explant inputs each for the negative control and morphogen treatments. As shown in FIG. 8, the ST1p_IPT_NOSp_ZmWUS2 construct elicited a significant (p-value=<0.00001, Fisher's exact test) 4-times increase over the negative control in the shooting %. In this experiment, there was some additional background of morphogenic response observed, although these instances were excluded from the tabulated data. As before, the 35Sp_IPT_NOSp_ZmWUS2 construct resulted in a decrease (to zero) in the shooting % compared to the negative control. The aforementioned data is shown in more detail in Table 4.

TABLE 4
Effects of Lead Promoters for Morphogen Expression on Shooting Responses in T0 Soybean Plants

| | | No. of | | Explants | % Shooting (Explants | |
|---|---|---|---|---|---|---|
| | Total | explants | % Explants | producing | regenerating transgenic | |
| Construct | explants | responding | responding | shoots | shoots/total explants) | Response |
| GFP Neg. Ctrl | 200 | 17 | 8.5 | 17 | 8.5 | Shoot |
| Constitutive | 200 | 7 | 3.5 | 0 | 0 | Morphogenic |
| ST1p-ZmWUS2 | 200 | 107 | 53.5 | 74 | 37 | Shoot (+Some |
| | | | | | | morphogenic background, |
| | | | | | | not counted) |

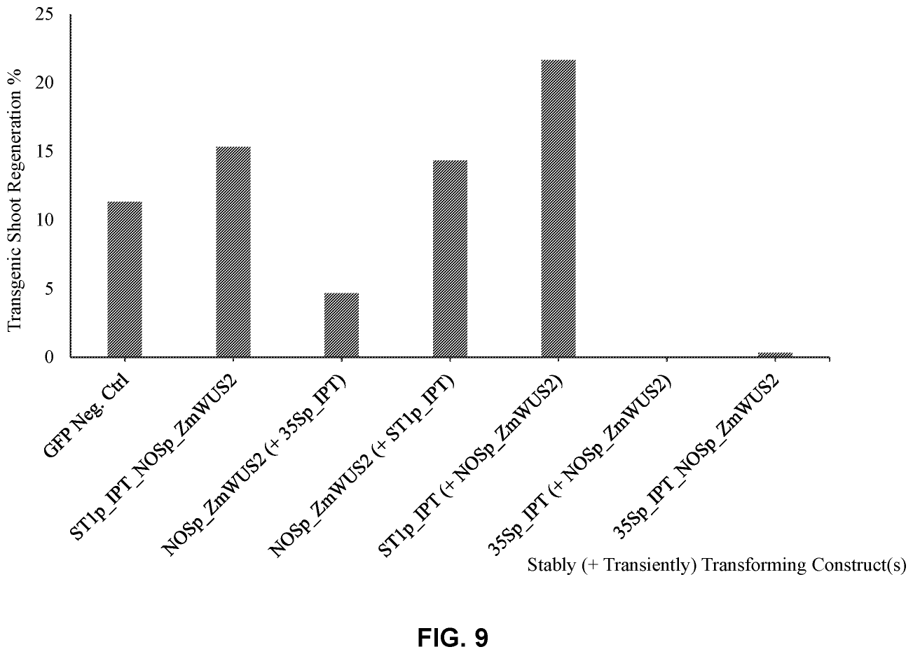

Another follow up experiment was conducted in another soybean genotype (Glycine max ‘Variety 2’), which included tested stable delivery of one morphogen construct, with transient delivery of the counterpart morphogen construct (shown in parentheses). As shown in FIG. 9, the stably transforming ST1p_IPT with transient NOSp_ZmWUS2 delivery elicited a significant (p-value=0.0009, Fisher's exact test) 1.9-times increase over the negative control in shooting %. In this particular experiment and/or genotype, the 35Sp_IPT_NOSp_ZmWUS2 construct did not elicit a significant difference (p-value=0.19, Fisher's exact test) from the negative control in the shooting %. The aforementioned data is shown in more detail in Table 5.

TABLE 5
Effects of Morphogen Expression and Delivery on Shooting Responses in T0 Soybean Plants

| | | No. of | | Explants | % Shooting (Explants | |
|---|---|---|---|---|---|---|
| | Total | explants | % Explants | producing | regenerating transgenic | |
| Description | explants | responding | responding | shoots | shoots/total explants) | Response |
| GFP Neg. Ctrl | 300 | 36 | 12 | 34 | 11.3 | Shoot |
| ST1p_IPT_NOSp_ZmWUS2 | 300 | 138 | 46 | 46 | 15.3 | Shoot |
| NOSp_ZmWUS2 (+35Sp_IPT) | 300 | 145 | 48.3 | 14 | 4.7 | Shoot |
| NOSp_ZmWUS2 (+ST1p_IPT) | 300 | 73 | 24.3 | 43 | 14.3 | Shoot |
| ST1p_IPT (+NOSp_ZmWUS2) | 300 | 87 | 29 | 65 | 21.7 | Shoot |
| 35Sp_IPT (+NOSp_ZmWUS2) | 300 | 183 | 61 | 0 | 0.0 | Morphogenic |
| 35Sp_IPT_NOSp_ZmWUS2 | 300 | 28 | 9.3 | 1 | 0.3 | Morphogenic |

TABLE 6
Sequence Descriptions

| SEQUENCE | |
|---|---|
| IDENTIFIER | DESCRIPTION |
| SEQ ID NO: 1 | Nucleic acid sequence for soybean XCP promoter - Glyma.04G014800 (ST1p) |
| SEQ ID NO: 2 | Nucleic acid sequence for soybean DUF1118 promoter - |
| | Glyma.04G161600 (ST2p) |
| SEQ ID NO: 3 | Nucleic acid sequence for soybean T5AH promoter - Glyma.18G052400 (ST3p) |
| SEQ ID NO: 4 | Nucleic acid sequence for pea XCP promoter - Psat4g084640 |
| SEQ ID NO: 5 | Nucleic acid sequence for pea XCP promoter - Psat5g008960 |
| SEQ ID NO: 6 | Nucleic acid sequence for medicago XCP promoter - Medtr3g116080 |
| SEQ ID NO: 7 | Nucleic acid sequence for pea DUF1118 promoter - Psat5g207080 |
| SEQ ID NO: 8 | Nucleic acid sequence for medicago DUF1118 promoter - Medtr3g026020 |
| SEQ ID NO: 9 | Nucleic acid sequence for pea T5AH promoter - Psat5g148400 |
| SEQ ID NO: 10 | Nucleic acid sequence for medicago T5AH promoter - Medtr3g467130 |
| SEQ ID NO: 11 | Nucleic acid sequence for medicago T5AH promoter - Medtr3g467140 |
| SEQ ID NO: 12 | Nucleic acid sequence for tomato XCP-LIKE promoter - Solyc12g094700 |
| SEQ ID NO: 13 | Nucleic acid sequence for Arachis hypogaea XCP-1 promoter - |
| | arahy.Tifrunner.gnm1.ann1.8AM4UR |
| SEQ ID NO: 14 | Nucleic acid sequence for Arachis hypogaea XCP-2 promoter - |
| | arahy.Tifrunner.gnm1.ann1.Q7CDUE |
| SEQ ID NO: 15 | Nucleic acid sequence for Cicer arietinum XCP-1 promoter - Ca_04803 |
| SEQ ID NO: 16 | Nucleic acid sequence for Cicer arietinum XCP-2 promoter - Ca_17491 |
| SEQ ID NO: 17 | Nucleic acid sequence for Lupinus albus XCP-1 promoter - Lalb_Chr23g0265531 |
| SEQ ID NO: 18 | Nucleic acid sequence for Lotus japonicus XCP-1 promoter - Lj1g0003774 |
| SEQ ID NO: 19 | Nucleic acid sequence for Phaseolus acutifolius XCP-1 promoter - |
| | Phacu.CVR.009G145500 |
| SEQ ID NO: 20 | Nucleic acid sequence for Phaseolus acutifolius XCP-2 promoter - |
| | Phacu.CVR.009G145300 |
| SEQ ID NO: 21 | Nucleic acid sequence for Phaseolus lunatus XCP-1 promoter - |
| | P109G0000016600.v1 |
| SEQ ID NO: 22 | Nucleic acid sequence for Phaseolus vulgaris XCP-1 promoter - |
| | Phvul.009G008200 |
| SEQ ID NO: 23 | Nucleic acid sequence for Phaseolus vulgaris XCP-2 promoter - |
| | Phvul.009G008100 |
| SEQ ID NO: 24 | Nucleic acid sequence for Trifolium pratense XCP-1 promoter - |
| | Tp57577_TGAC_v2_gene38208 |
| SEQ ID NO: 25 | Nucleic acid sequence for Trifolium pratense XCP-2 promoter - |
| | Tp57577_TGAC_v2_gene15758 |
| SEQ ID NO: 26 | Nucleic acid sequence for Vigna unguiculata XCP-1 promoter - Vigun09g263200 |
| SEQ ID NO: 27 | Nucleic acid sequence for Vigna unguiculata XCP-2 promoter - Vigun09g263100 |
| SEQ ID NO: 28 | Nucleic acid sequence for soybean DUF1118 exon 1 - Glyma.04G161600 |
| SEQ ID NO: 29 | Nucleic acid sequence for soybean DUF1118 intron - Glyma.04G161600 |
| SEQ ID NO: 30 | Nucleic acid sequence for pea DUF1118 exon 1 - Psat5g207080 |
| SEQ ID NO: 31 | Nucleic acid sequence for pea DUF1118 intron - Psat5g207080 |
| SEQ ID NO: 32 | Nucleic acid sequence for medicago DUF1118 exon 1 - Medtr3g026020 |
| SEQ ID NO: 33 | Nucleic acid sequence for medicago DUF1118 intron - Medtr3g026020 |
| SEQ ID NO: 34 | Nucleic acid sequence for soybean T5AH exon 1 - Glyma.18G052400 |
| SEQ ID NO: 35 | Nucleic acid sequence for soybean T5AH intron - Glyma.18G052400 |
| SEQ ID NO: 36 | Nucleic acid sequence for pea T5AH exon 1 - Psat5g148400 |
| SEQ ID NO: 37 | Nucleic acid sequence for pea T5AH intron - Psat5g148400 |
| SEQ ID NO: 38 | Nucleic acid sequence for medicago T5AH exon 1 - Medtr3g467130 |
| SEQ ID NO: 39 | Nucleic acid sequence for medicago T5AH intron - Medtr3g467130 |
| SEQ ID NO: 40 | Nucleic acid sequence for medicago T5AH exon 1 - Medtr3g467140 |
| SEQ ID NO: 41 | Nucleic acid sequence for medicago T5AH intron - Medtr3g467140 |
| SEQ ID NO: 42 | Nucleic acid sequence for 35Sp |
| SEQ ID NO: 43 | Nucleic acid sequence for NOSp |
| SEQ ID NO: 44 | Amino acid sequence for ISOPENTYL TRANSFERASE (IPT) |
| SEQ ID NO: 45 | Amino acid sequence for Zea mays WUSCHEL 2 (ZmWUS2) |

Although the foregoing invention has been described in some detail by way of illustration and example for purposes of clarity of understanding, it will be obvious that certain changes and modifications may be practiced within the scope of the appended claims.

What is claimed is:

1. A DNA construct comprising, in operable linkage:
(a) a first promoter molecule comprising a nucleic acid sequence that shares at least 80% sequence identity with any one of SEQ ID NOs: 1-27 and retains transcription initiation activity, or a nucleic acid sequence of any one of SEQ ID NOs: 1-27; and
(b) a first polynucleotide of interest.

2. The DNA construct of claim 1, wherein the first polynucleotide of interest encodes a morphogen, a transforming protein, a recombination modulator, a repair modulator, a defense pathway modulator, a nuclease, a selectable marker, and/or a regulatory RNA.

3. The DNA construct of claim 1 or 2, wherein said first polynucleotide of interest comprises a nucleic acid sequence that encodes: (i) a polypeptide comprising an amino acid sequence having at least 80% sequence identity to SEQ ID NO: 44 or SEQ ID NO: 45, wherein said polypeptide has morphogenic activity; or (ii) a polypeptide comprising an amino acid sequence of SEQ ID NO: 44 or SEQ ID NO: 45.

4. The DNA construct of claim 2, wherein the regulatory RNA is one or more of a microRNA (miRNA), a short-hairpin RNA, a guide RNA, a transposase, a homology-directed repair enhancer, and a non-homologous end-joining suppressor.

5. The DNA construct of any one of claims 1-4, wherein the promoter molecule further comprises a 5′UTR sequence, a 5′UTR intron sequence, an exon sequence from a coding region, and/or an intron sequence from a coding region of a plant genome.

6. The DNA construct of any one of claims 1-5, further comprising a second promoter molecule operably linked to a second polynucleotide of interest.

7. The DNA construct of claim 6, wherein the first and second polynucleotides of interest each encode a morphogen.

8. The DNA construct of claim 7, wherein the first polynucleotide of interest operably linked to the first promoter molecule comprises a nucleic acid sequence that encodes: (i) a polypeptide comprising an amino acid sequence that shares at least 80% sequence identity with SEQ ID NO: 44, wherein said polypeptide has morphogenic activity, or (ii) a polypeptide comprising an amino acid sequence of SEQ ID NO: 44; and
wherein the second polynucleotide of interest operably linked to the second promoter molecule comprises a nucleic acid sequence that encodes: (iii) a polypeptide comprising an amino acid sequence that shares at least 80% sequence identity with SEQ ID NO: 45, wherein said polypeptide has morphogenic activity, or (iv) a polypeptide comprising an amino acid sequence of SEQ ID NO: 45.

9. The DNA construct of claim 7, wherein the first polynucleotide of interest operably linked to the first promoter molecule comprises a nucleic acid sequence that encodes: (i) a polypeptide comprising an amino acid sequence that shares at least 80% sequence identity with SEQ ID NO: 45, wherein said polypeptide has morphogenic activity, or (ii) a polypeptide comprising an amino acid sequence of SEQ ID NO: 45; and
wherein the second polynucleotide of interest operably linked to the second promoter molecule comprises a nucleic acid sequence that encodes: (iii) a polypeptide comprising an amino acid sequence that shares at least 80% sequence identity with SEQ ID NO: 44, wherein said polypeptide has morphogenic activity, or (iv) a polypeptide comprising an amino acid sequence of SEQ ID NO: 44.

10. The DNA construct of any one of claims 6-9, wherein the first promoter molecule comprises a nucleic acid sequence that shares at least 80% sequence identity with any one of SEQ ID NOs: 1-3 and retains transcription initiation activity, or a nucleic acid sequence of any one of SEQ ID NOs: 1-3.

11. The DNA construct of any one of claims 6-10, wherein the second promoter molecule comprises a nucleic acid sequence that shares at least 80% sequence identity with any one of SEQ ID NOs: 1-27 and retains transcription initiation activity, or a nucleic acid sequence of any one of SEQ ID NOs: 1-27.

12. The DNA construct of any one of claims 1-11, wherein said promoter molecule(s) comprising a nucleic acid sequence that shares at least 80% sequence identity with any one of SEQ ID NOs: 1-27 and retains transcription initiation activity, or a nucleic acid sequence of any one of SEQ ID NOs: 1-27, initiates expression of the operably linked polynucleotide of interest limited to a seed-to-seedling developmental phase when the DNA construct is introduced in a plant or plant part.

13. The DNA construct of any one of claims 1-12, wherein said promoter molecule(s) comprising a nucleic acid sequence that shares at least 80% sequence identity with any one of SEQ ID NOs: 1-27 and retains transcription initiation activity, or a nucleic acid sequence of any one of SEQ ID NOs: 1-27, initiates embryonic tissue-preferred expression of the operably linked polynucleotide of interest when the DNA construct is introduced in a plant or plant part.

14. The DNA construct of claim 13, wherein the preferred embryonic tissue is epicotyl, hypocotyl, radicle, cotyledon, or a combination thereof.

15. A cell comprising the DNA construct of any one of claims 1-14.

16. The cell of claim 15, wherein the cell is a plant cell.

17. A plant or plant part comprising the DNA construct of any one of claims 1-14 or the cell of claim 15 or 16.

18. The plant or plant part of claim 17, wherein said plant is selected from the group consisting of corn (Zea mays), Brassica species, Brassica napus, Brassica rapa, Brassica juncea, alfalfa (Medicago sativa), pea (Pisum sativum), fava bean (Vicia faba), common bean (Phaseolus vulgaris), chickpea (Cicer arietinum), mung bean (Vigna radiata), white lupin (Lupinus albus), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

19. The plant or plant part of claim 17 or 18, wherein said plant is a dicot.

20. The plant or plant part of claim 19, wherein said plant is a legume.

21. A method of expressing a polynucleotide of interest in a plant or plant part comprising introducing a DNA construct into said plant or plant part, wherein the DNA construct comprises, in operable linkage:
(a) a first promoter molecule comprising a nucleic acid sequence that shares at least 80% sequence identity with any one of SEQ ID NOs: 1-27 and retains transcription initiation activity, or a nucleic acid sequence of any one of SEQ ID NOs: 1-27; and
(b) a first polynucleotide of interest.

22. The method of claim 21, said method comprising:
introducing a DNA construct into a plant cell, wherein the DNA construct comprises, in operable linkage: (a) a first promoter molecule comprising a nucleic acid sequence that shares at least 80% sequence identity with any one of SEQ ID NOs: 1-27 and retains transcription initiation activity, or a nucleic acid sequence of any one of SEQ ID NOs: 1-27; and (b) a first polynucleotide of interest; and
regenerating a transformed plant or plant part from said plant cell.

23. The method of claim 22, wherein the method increases normal shoot formation, frequency of shoot producing plants or plant parts, and/or number of regenerated shoots from transformed plants or plant parts relative to a control method comprising introducing a control DNA construct comprising a control promoter molecule into a plant cell.

24. The method of claim 23, wherein the frequency of shoot producing plants or plant parts is increased by about 10% to about 500% relative to a control method.

25. The method of claim 23, wherein the number of regenerated shoots from transformed plants or plant parts is increased by about 10% to about 1200% relative to a control method.

26. The method of any one of claims 21-25, wherein the first polynucleotide of interest encodes a morphogen, a transforming protein, a recombination modulator, a repair modulator, a defense pathway modulator, a nuclease, a selectable marker, and/or a regulatory RNA.

27. The method of claim 26, wherein said first polynucleotide of interest comprises a nucleic acid sequence that encodes: (i) a polypeptide comprising an amino acid sequence having at least 80% sequence identity to SEQ ID NO: 44 or SEQ ID NO: 45, wherein said polypeptide has morphogenic activity; or (ii) a polypeptide comprising an amino acid sequence of SEQ ID NO: 44 or SEQ ID NO: 45.

28. The method of claim 26, wherein the regulatory RNA is one or more of a microRNA (miRNA), a short-hairpin RNA, a guide RNA, a transposase, a homology-directed repair enhancer, and a non-homologous end-joining suppressor.

29. The method of any one of claims 21-28, wherein the first promoter molecule further comprises a 5′UTR sequence, a 5′UTR intron sequence, an exon sequence from a coding region, and/or an intron sequence from a coding region of a plant genome.

30. The method of any one of claims 21-29, wherein the DNA construct further comprises a second promoter molecule operably linked to a second polynucleotide of interest.

31. The method of any one of claims 21-29, further comprising introducing a second DNA construct into said plant or plant part, wherein the second DNA construct comprises a second promoter molecule operably linked to a second polynucleotide of interest.

32. The method of claim 30 or 31, wherein the first and second polynucleotides of interest each encode a morphogen.

33. The method of claim 32, wherein the first polynucleotide of interest operably linked to the first promoter molecule comprises a nucleic acid sequence that encodes: (i) a polypeptide comprising an amino acid sequence that shares at least 80% sequence identity with SEQ ID NO: 44, wherein said polypeptide has morphogenic activity, or (ii) a polypeptide comprising an amino acid sequence of SEQ ID NO: 44; and
wherein the second polynucleotide of interest operably linked to the second promoter molecule comprises a nucleic acid sequence that encodes: (iii) a polypeptide comprising an amino acid sequence that shares at least 80% sequence identity with SEQ ID NO: 45, wherein said polypeptide has morphogenic activity, or (iv) a polypeptide comprising an amino acid sequence of SEQ ID NO: 45.

34. The method of claim 32, wherein the first polynucleotide of interest of the first DNA construct comprises a nucleic acid sequence that encodes: (i) a polypeptide comprising an amino acid sequence having at least 80% sequence identity to SEQ ID NO: 45, wherein said polypeptide has morphogenic activity; or (ii) a polypeptide comprising an amino acid sequence of SEQ ID NO: 45; and
wherein the second polynucleotide of interest of the second DNA construct comprises a nucleic acid sequence that encodes: (iii) a polypeptide comprising an amino acid sequence having at least 80% sequence identity to SEQ ID NO: 44, wherein said polypeptide has morphogenic activity; or (iv) a polypeptide comprising an amino acid sequence of SEQ ID NO: 44.

35. The method of any one of claims 21-34, wherein the first promoter molecule comprises a nucleic acid sequence that shares at least 80% sequence identity with any one of SEQ ID NOs: 1-3 and retains transcription initiation activity, or a nucleic acid sequence of any one of SEQ ID NOs: 1-3.

36. The method of any one of claims 30-35, wherein the second promoter molecule comprises a nucleic acid sequence that shares at least 80% sequence identity with any one of SEQ ID NOs: 1-27 and retains transcription initiation activity, or a nucleic acid sequence of any one of SEQ ID NOs: 1-27.

37. The method of any one of claims 21-36, wherein said promoter molecule(s) comprising a nucleic acid sequence that shares at least 80% sequence identity with any one of SEQ ID NOs: 1-27 and retains transcription initiation activity, or a nucleic acid sequence of any one of SEQ ID NOs: 1-27, initiates expression of the operably linked polynucleotide of interest limited to a seed-to-seedling developmental phase in the plant or plant part.

38. The method of any one of claims 21-37, wherein the said promoter molecule(s) comprising a nucleic acid sequence that shares at least 80% sequence identity with any one of SEQ ID NOs: 1-27 and retains transcription initiation activity, or a nucleic acid sequence of any one of SEQ ID NOs: 1-27, initiates embryonic tissue-preferred expression of the operably linked polynucleotide of interest in the plant or plant part.

39. The method of claim 38, wherein the preferred embryonic tissue is epicotyl, hypocotyl, radicle, cotyledon, or a combination thereof.

40. The method of any one of claims 21-39, wherein said plant is selected from the group consisting of corn (Zea mays), Brassica species, Brassica napus, Brassica rapa, Brassica juncea, alfalfa (Medicago sativa), pea (Pisum sativum), fava bean (Vicia faba), common bean (Phaseolus vulgaris), chickpea (Cicer arietinum), mung bean (Vigna radiata), white lupin (Lupinus albus), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

41. The method of any one of claims 21-40, wherein said plant is a dicot.

42. The method of claim 41, wherein said plant is a legume.

43. The method of any one of claims 21-42, wherein the polynucleotide(s) of interest is stably inserted into a genome of said plant or plant part.

44. A plant or plant part produced by the method of any one of claims 21-43, wherein said plant or plant part comprises said DNA construct.
